(19) United States
(12) Patent Application Publication
(10) Pub. No.: US 2024/0294988 A1
(43) Pub. Date: Sep. 5, 2024

(54) METHODS OF DETERMINING RESPONSIVENESS TO CHEMOTHERAPEUTIC COMPOUNDS FOR CANCER THERAPY

(71) Applicants: Northwestern University, Evanston, IL (US); President and Fellows of Harvard College, Cambridge, MA (US)

(72) Inventors: Crismita Clement Dmello, Evanston, IL (US); Adam Mendel Sonabend Worthalter, Evanston, IL (US); Aaron Sonabend, Cambridge, MA (US); Roger Stupp, Evanston, IL (US)

(21) Appl. No.: 18/572,654

(22) PCT Filed: Jun. 23, 2022
(86) PCT No.: PCT/US22/73125

§ 371 Date: Dec. 20, 2023

Related U.S. Application Data
(60) Provisional application No. 63/202,761, filed on Jun. 23, 2021.

(51) Int. Cl. C12Q 1/6886; A61K 45/00; G01N 33/574

(52) U.S. Cl. CPC C12Q 1/6886; A61K 45/00; G01N 33/57484; C12Q 2600/158

(57) ABSTRACT

Disclosed herein are methods for identifying cancers that are susceptible to tubulin inhibitor chemotherapeutics. In some embodiment, the methods include determining the level of expression of one or more biomarkers in a tumor sample selected from signal sequence receptor 3 (SSR3), interleukin-1 receptor-associated kinase 4 (IRAK4), transmembrane protein 131 (TMEM131), enhancer of polycomb homolog 2 (EPC2), muscleblind like splicing regulator 1 (MBNL1), zinc finger protein 813 (ZNF813), zinc finger and BTB domain containing 20 (ZBTB20). In some embodiments, the biomarker expression level is above a threshold, baseline, or control expression level. In some embodiments, such cancers are treated with a tubulin inhibitor.

### CROSS-REFERENCE TO RELATED APPLICATIONS

The present application claims priority to U.S. Provisional Patent Application 63/202,761, filed Jun. 23, 2021, the entire contents of which are hereby incorporated by reference.

### STATEMENT REGARDING FEDERALLY SPONSORED RESEARCH

N/A

### REFERENCE TO A SEQUENCE LISTING

This application is being filed electronically via EFS-Web and includes an electronically submitted Sequence Listing in .txt format. The .txt file contains a sequence listing entitled “702581.02181 ST25” created on Jun. 20, 2022 and is 69,586 bytes in size. The Sequence Listing contained in this .txt file is part of the specification and is hereby incorporated by reference herein in its entirety.

### BACKGROUND OF THE INVENTION

Paclitaxel (PTX) and taxanes are among the most commonly used chemotherapeutic drugs for cancer. These agents are exquisitely potent, exerting cytotoxicity to cancer cells at nanomolar concentrations. Thus, PTX is the basis of chemotherapeutic regimens for metastatic breast, pancreatic, lung and ovarian carcinomas (Barkat et al., 2019).

Gliomas and glioblastoma (GBM) in particular, are the most common and malignant of all primary brain tumors in adults. Unfortunately, in spite of extensive research and the use of multimodal therapeutic strategies, the median overall survival time is 20 months from diagnosis (Stupp et al., 2017). PTX is one of the most potent drugs against GBM, with an IC50 1400-fold lower temozolomide for these tumors (Yang et al., 2013). The poor penetration of PTX across the blood-brain barrier (BBB) has limited its use for brain tumors (Chamberlain and Kormanik, 1995), yet there are multiple emerging strategies to deliver PTX into the brain (Gradishar et al., 2005; Kumthekar et al., 2020; Zhang et al., 2020). We recently showed that albumin-bound PTX is safe, exhibits brain penetration at therapeutic levels with ultrasound (US)-based BBB disruption, and is efficacious for glioma xenografts (Zhang et al., 2020). Given this, we are initiating a Phase 1-2 clinical trial for recurrent GBM, to evaluate the safety and efficacy of delivering PTX using skull-implantable ultrasound device that transiently opens the BBB (NCT04528680)

PTX is highly potent, yet there is a spectrum of susceptibility to this drug within individual tumors of a given cancer. This is particularly important for GBM, as these tumors are notorious for their molecular heterogeneity and unpredictable response to therapies. Thus, even if the challenge of PTX delivery across the BBB is solved, the efficacious implementation of PTX-based therapy for GBM will be greatly influenced by identification of which tumors will be responsive to this drug. Despite the wide-spread use of PTX for breast cancer and our approach to repurpose it for GBM, there are no predictive biomarkers to inform which patients will benefit from this therapy. Understanding the biological basis of individual response to this drug, and development of predictive biomarkers will allow a personalized implementation of PTX-based therapy for GBM, and refine the indication for using this drug in common scenarios such as metastatic breast cancer in which paclitaxel is readily used.

Many independent groups have made several efforts to identify predictive biomarker for PTX using different approaches. Rouzier et al. and colleagues have identified expression of microtubule associated protein tau in conferring resistance to PTX treatment in breast cancer using U133A chips. They showed that knockdown of tau protein rendered the cells susceptible to PTX (Rouzier et al., 2005). However, the NSABP-B 28 Randomized Clinical Trial failed to show any interaction between tau expression and benefit from PTX (Pusztai et al., 2009). Another study by Swanton et al. and colleagues using a kinome and ceramidome siRNA screen, identified the role of ceramide transport protein, COL4A3BP in conferring resistance to PTX in ovarian cancer setting (Swanton et al., 2007). The PTX resistant gene signature identified from this study was successfully validated in patients with triple-negative breast cancer (Juul et al., 2010). Whole genome siRNA screen was performed by Whitehurst and colleagues to identify chemosensitizer for PTX i.e. to identify gene targets that specifically reduce cell viability in the presence of sublethal concentrations of PTX. They demonstrated that inhibition of proteasomal subunits chemosensitizes lung cancer cells to PTX (Whitehurst et al., 2007). In an independent study MCL1 expression was shown to confer resistance to antitubulin chemotherapeutics like PTX and vincristine (Wertz et al., 2011). Another group has reported the role of solute carrier transporters in conferring resistance to PTX by mediating efflux of the drug (Njiaju et al., 2012). Cyclin G1 levels were shown to determine resistance to PTX and ovarian cancer patients with cyclin G1 amplification manifested poor post-surgery survival in a taxane treated group (Russell et al., 2012). Yet, none of these studies has led to a clinically useful biomarker/s that predicts resistance/response to PTX/taxane treatment.

### BRIEF SUMMARY OF THE INVENTION

Disclosed herein are methods for identifying cancers that are susceptible to tubulin inhibitor chemotherapeutics. In some embodiment, the methods include determining the level of expression of one or more biomarkers in a tumor sample, wherein the biomarker comprises signal sequence receptor 3 (SSR3), interleukin-1 receptor-associated kinase 4 (IRAK4), transmembrane protein 131 (TMEM131), enhancer of polycomb homolog 2 (EPC2), muscleblind like splicing regulator 1 (MBNL1), zinc finger protein 813 (ZNF813), zinc finger and BTB domain containing 20 (ZBTB20). In some embodiments, the biomarker is SSR3. In some embodiments, the biomarker expression level is above a threshold, baseline, or control expression level. In some embodiments, such cancers are treated with a tubulin inhibitor. In some embodiments, the tubulin inhibitor includes one or more of taxane, paclitaxel, docetaxel, abraxane, and taxotere, or vinca alkaloids, such as vinorelbine, vinblastine, vincristine, and vindesine. In some embodiments, the cancer is breast cancer, ovarian cancer, Kaposi's sarcoma, or glioblastoma, and the tumor is a breast cancer tumor, an ovarian cancer tumor, a Kaposi's sarcoma tumor, or a glioma, such as glioblastoma. In some embodiments, the tumor is a glioblastoma and the method comprises administering the tubulin inhibitor to the glioblastoma via transient blood brain barrier (BBB) opening. In some embodiments, the transient BBB opening is achieved via administering ultrasound treatment and concomitantly administering microbubbles.

### BRIEF DESCRIPTION OF THE DRAWINGS

FIG. 1A-1F. Whole genome CRISPR screen reveals genes that confer susceptibility to PTX treatment. (A) PTX dose response curve using glioma cell lines. H4 cell line showed the highest degree of sensitivity to PTX hence was selected for the PTX CRISPR screen. (B) Schematic representation-H4 cells were transduced with a genome-scale gRNA library (four gRNAs/gene). The edited cells were called as (Day 0). These cells were subjected to PTX (0.025 μM) or vehicle (DMSO) treatment. Cells from both these groups were sampled at D14 and D21 for Illumina sequencing of gRNA. (C) Graphical representation depicting progress of the screen in PTX treated and DMSO control groups. Two distinct phases were noted as the screen progressed, in the PTX treated group. First phase or selection phase was seen where the sensitive clones were eliminated from the screen and the second phase or expansion phase was marked by a steep outgrowth of the resistant clones even in the presence of PTX. (D) Deseq analysis shows top genes (orange) for enriched gRNAs from PTX screen. Dashed line indicates a p<0.05. Grey dots are all the genes with p>0.05. (E) sgRSEA analysis shows top genes (orange) for enriched gRNAs from PTX screen. Dashed line indicates a p<0.05. Grey continuous line like dots are all the genes with p>0.05. (F) Significantly enriched pathways along with their corresponding genes, selected in the PTX CRISPR screen.

FIG. 2A-2D. Cox Proportional-Hazards Model (Cox) identifies genes predictive of survival in breast cancer patients from the CRISPR screen gene list. (A) Schematic illustration of the pipeline developed to select the biomarkers for PTX susceptibility. The highly enriched genes from the CRISPR screen were filtered through the TCGA breast cancer dataset and genes with lowest HR and significant p value at the 0.05 level were validated using single gene KO in breast cancer and glioma cell lines. (B) Scatter plot showing HR and −log₁₀ p value of interaction term for the genes that were found to predict survival in the TCGA taxane treated breast cancer patients as compared to patients who did not receive any chemotherapy. (C) Scatter plot showing HR and −log₁₀ p value of interaction for the genes that were found to predict survival in the TCGA taxane treated breast cancer patients as compared to patients who received non-taxane chemotherapies. (D) Percentage of predictive genes from the CRISPR screen gene list in the TCGA taxane treated breast cancer vs. patients who did not receive any chemotherapy (p<0.0003) and in the TCGA taxane treated breast cancer vs. patients who received non-taxane chemotherapies (p<0.0023). P-values are computed with bootstrap analysis.

FIG. 3A-3D. SSR3 mRNA levels correlate with response to taxanes in breast cancer patients. (A) Kaplan-Meier survival analysis showing the association of high SSR3 mRNA levels with favorable survival outcomes in TCGA taxane treated breast cancer patients and not in patients who did not receive any chemotherapy or who received non-taxane chemotherapies. (B) Kaplan-Meier survival analysis showing the association of high SSR3 expression with relapse-free survival in the GSE25066 dataset which includes only taxane-treated patients who have not received hormonal therapy. Kaplan-Meier survival analysis showing no such association in combined GEO datasets (GSE16716, GSE19615, GSE31519, GSE37946, GSE45255 and GSE65194) which includes patients who have not received hormonal therapy or chemotherapy. For (A) and (B) median was used as a cutoff to separate high and low SSR3 expression. The HR and the p value were determined by the Cox model. Kaplan-Meier survival analysis showing the survival outcome of (C) GDC TCGA GBM and (D) GDC TCGA breast cancer patients with high and low SSR mRNA expression. Median was used as a cutoff to separate high and low SSR3 mRNA expression.

FIG. 4A-4E. Baseline SSR3 expression predicts susceptibility to PTX in human glioma explant cultures. (A) Schematic depicting the experimental design wherein one piece of the freshly resected human GBM tumor sample was stained for SSR3 using IHC and the other piece of the same tumor was cultured in-vitro to develop primary explant culture (n=14). (B) Representative H and E staining and immunostaining of high and low SSR3 expressing human GBMs that were freshly resected. Scale bar in black=250 μm. (C) Graph showing PTX AUC for the individual tumor explants derived from the freshly resected human GBM sample tissue. (D) Scatter plot showing correlation between susceptibility to PTX (AUC) of the individual explant cultures and SSR3 staining intensity of the paired tumor tissue (by quantitative IHC measurement). p values, two-tailed t test. Data is presented as mean±s.d. Each dot represents an independent patient sample. (E) Kaplan-Meier curve comparing overall survival of human GBM patients (from Northwestern medicine hospital (NMH)) defined as either high or low SSR3 expressing by quantitative IHC measurement. Representative H and E staining and SSR3 immunostaining of SSR3 high and low tumor samples from GBM patients. Scale bar in black=250 μm. Median was used as a cutoff to separate high and low SSR3 protein expression in (no./mm²).

FIG. 5A-5D. SSR3 expression positively correlates with response to paclitaxel in glioma, breast cancer cells and in intracranial glioma models. (A) Western blot showing SSR3 protein levels for the indicated glioma PDX cell lines. PTX dose response curves for the glioma PDX cell lines. Scatter plot showing correlation between susceptibility to PTX (AUC) and protein levels of SSR3 as quantified by Image J. (B) Western blot showing SSR3 protein levels for the indicated breast cancer cell lines. PTX dose response curves for the breast cancer cell lines. Scatter plot showing correlation between susceptibility to PTX (AUC) and protein levels of SSR3 as quantified by Image J. For (A) and (B)—p values, two-tailed paired t test. Data is presented as mean±s.e. (C) Western blot showing baseline protein levels of SSR3 in the MES83, GBM12, GBM6 and TS543 cell lines. Schematic representation of the dosing scheme followed to treat mice having intracranial tumors with albumin bound PTX formulation-Abraxane. Representative H and E staining and immunostaining of SSR3 on the tumor tissues from the xenografts developed from these cell lines. Scale bar in black=250 μm. Scale bar in the inset=50 μm. The survival curve alongside, shows the survival of the mice injected with these cell lines in ABX treated or control treated groups. Log-rank analysis was used to determine the survival differences. For MES83, median survival was 20 for control vs. 31 days for ABX treated group, p=0.0041, (n=12). For GBM12, median survival was 24 for control vs. 38 days for ABX treated group, p<0.0001, (n=10). For GBM6, median survival was 29 for control vs. 30 days for ABX treated group, p=0.77, n=10. For TS543, median survival was 38.5 for control vs. 43 days for ABX treated group, p=0.0050, (n=10) for control and (n=9) for ABX treated group. ****p<0.0001 and **p<0.01. (D) Scatter plot showing correlation between baseline SSR3 protein expression (no./mm2) in xenograft tumor tissues (n=3/group) and percent increase in median survival.

FIG. 6A-6E. SSR3 protein levels determine susceptibility to PTX in breast and GBM cell lines. (A) The graph shows SSR3 mRNA expression in GBM and breast cancers in relation to its non-tumor regions from TCGA GBM and breast cancer datasets respectively. (B) The graph shows the fold enrichment of gRNA's for SSR3 in PTX treated group as compared to DMSO control group in the CRISPR screen. No change is seen in the enrichment of control gRNA's (non-targeting gRNA). p values, two-tailed t test. PTX dose response curves and western blots for SSR3 gene KO clones derived from (C) H4 glioma cells and (D) MDA-MB 468 breast cancer cells. (E) PTX dose response curve for SSR3 overexpressing cells derived from GBM6 glioma PDX cell line. Western blot showing overexpression of SSR3 in GBM6 cells.

FIG. 7A-7F. SSR3 protein levels determine response to PTX in in-vivo glioma and breast cancer models

(A) Schematic representation of the dosing scheme followed to treat mice having intracranial tumors with ABX. (B and C) Representative H and E staining and immunostaining of SSR3 on the tumor tissues from the xenografts developed from GBM6 vector control and SSR3 overexpressing cells respectively. Scale bar in black=100 μm. Kaplan-Meier survival curves for mice injected with vector control (GBM6 VBB) and GBM6 SSR3 overexpressing (GBM6 SSR3 O/E) clones—with and without the treatment with Abraxane. Log-rank analysis was used to determine the survival differences. For GBM6 VBB, median survival was 47 for control vs. 50 days for ABX treated group, p<0.01, number of mice (n=10/group). For GBM6 SSR3 O/E, median survival was 29 for control vs. 38 days for ABX treated group, p<0.0016 (n=10/group). **p<0.01, *p<0.05. (D) Schematic representation of the dosing scheme followed to treat mice bearing mammary fat pad tumors with ABX. (E) Representative images of mice bearing mammary fat pad tumors-injected on one side with MDA-MB-468 vector control (NTC) and on contralateral side with SSR3 KO (SSR3 KO1) clones. The mice were randomized on day 30 when most of the tumors measured around 62.5 mm³. Single dose of ABX (10 mg/kg) was administered to the mice on day 30 and PBS was administered to the control group. (F) Tumors were measured once every week after the drug treatment (n=5/group). **p<0.01, *p<0.05.

FIG. 8A-8E. SSR3 mediated susceptibility to PTX seems to be regulated through phosphorylation of IRE1α in glioma cells. (A) Western blot showing pIRE1α and SSR3 protein levels in glioma PDX cell lines. Scatter plot showing correlation between SSR3 and pIRE1α protein levels, as quantified by Image J. (B) Western blot showing p-IRE1α levels in PTX treated H4 SSR3 KO and vector control clones. Western blot and PTX dose response curve for IRE1α gene KO clones derived from H4 glioma cells. (C) Western blot showing p-IRE1α levels in GBM6 SSR3 overexpressing and vector control clones. Western blot and PTX dose response curve of IRE1α gene KO clones and its vector control cells derived from GBM6 SSR3 overexpressing cells. PTX dose response curve for GBM6 SSR3 overexpressing and GBM6 parental cells is plotted for comparison. (D) IRE1α knockout in wildtype GBM6 cells does not change its susceptibility to PTX Western blot and PTX dose response curve for IRE1α gene KO clones derived from wildtype GBM6 cells. (E) Schematic showing SSR3 mediated stabilization of phosphorylated p-IRE1α to induce cell death in the presence of PTX.

FIG. 9A-9D. S1. Paclitaxel exhibits a broad spectrum of susceptibility across breast cancer and glioma cell lines. (A) Comparison of IC50 drug concentrations of the commonly used chemotherapeutics against glioma cell lines from Sanger/CCLE database (n=24). (B) Comparison of PTX IC50 concentrations against all the available cancer cell lines from Sanger/CCLE database. Marked with black boxes are the IC50 drug concentrations for PTX for the cancers treated with PTX (n=268). Marked with red boxes are low grade gliomas (LGG) and glioblastomas (GBM). Histogram showing PTX IC50 drug concentrations for (C) breast cancer and (D) glioma cell lines from Sanger database (https://www.cancerrxgene.org/). The dotted line indicates the brain PTX concentration (0.3-0.4 μM) that can be achieved by US-mediated delivery of PTX in the mouse brain (Zhang et al., 2020).

FIG. 10. S2. Optimization of immunohistochemistry staining technique for the SSR3 antibody. Titration of the SSR3 antibody ((HPA014906)) using different dilutions on GBM samples included in the study. The antibody titration was performed on NU00884 GBM tissue sections. Here we show images of a same region of NU00884 stained with the indicated dilutions of the SSR3 antibody. Dot plot representing SSR3 protein expression of all the GBM tissues stained using 1:200 dilution of SSR3 antibody.

FIG. 11A-11B. S3. Gene expression profiles of putative biomarkers for paclitaxel susceptibility. Baseline RNA expression profile for all the 9 genes shortlisted by overlapping CRISPR screen and TCGA taxane treated breast cancer data in (A) TCGA gliomas and (B) TCGA breast cancers, along with their non-tumor counterparts.

FIG. 12A-12B. S4. Phenotypic validation of potential PTX biomarkers using single gene knockouts in H4 glioma cell line. (A) Histogram alongside each dose response curve shows the fold change in the expression of individual gene KO as compared to its non-targeting control, as determined using quantitative real time PCR. The gene name corresponding to the knockout is indicated in the figure. (B) The histogram shows the PTX AUC values for the single gene KO generated in H4 glioma cell line. Data is presented as mean±s.e. One-way Anova multiple comparisons-method was used to analyze the differences between single gene knockout as compared to its non-targeting control (NTC)—****p<0.0001, ***p<0.001, **p<0.01, *p<0.05.

FIG. 13A-13B. S5 Phenotypic validation of potential PTX biomarkers using single gene knockouts in MDA-MB 468 breast cancer cell line. (A) Histogram shows the fold change in the expression of individual gene KO as compared to its non-targeting control, as determined using quantitative real time PCR. The gene name corresponding to the knockout is indicated in the figure. (B) The histogram shows the PTX AUC values for the single gene KO generated in H4 glioma cell line. Data is presented as mean±s.e. One-way Anova-multiple comparisons method was used to analyze the differences between single gene knockout as compared to its non-targeting control (NTC)—****p<0.0001, ***p<0.001, **p<0.01, *p<0.05.

FIG. 14A-14E. S6. Correlation between SSR3 and ER stress markers in glioma PDX cell lines. (A) Western blot showing SSR3 protein levels upon treatment with 0.05 μM and 0.5 μM PTX in H4 NTC, MES83 and GBM6 cells. (B) Western blot showing SSR3, BIP, IRE1α and PERK protein levels in glioma PDX cell lines. Scatter plot showing correlation between (C) SSR3 and BIP protein levels, (D) SSR3 and IRE1α protein levels (E) SSR3 and PERK protein levels, as quantified by Image J.

FIG. 15. S7. SSR3 mediated susceptibility to PTX is regulated by mechanism/s other than tubulin stabilization. Representative confocal images of SSR3 and α-tubulin staining in H4 SSR3 KO and control cells under PTX (0.5 μM) or vehicle treated conditions for 48 h. Scale bar in white=10 μm. Histogram alongside shows the number of microtubule bundles per cell cluster. 5 random fields from two independent experiments were counted for the analysis. One-way Anova-multiple comparisons method was used for the analysis-***p<0.001, **p<0.01.

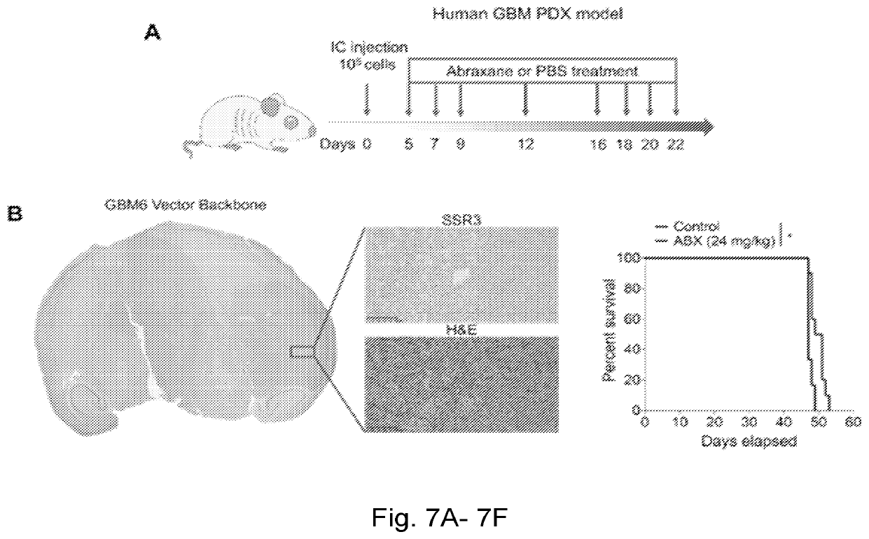

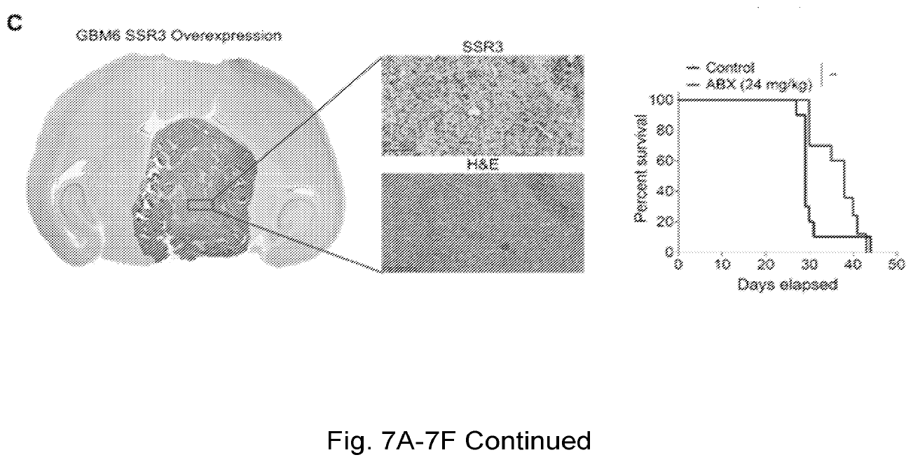

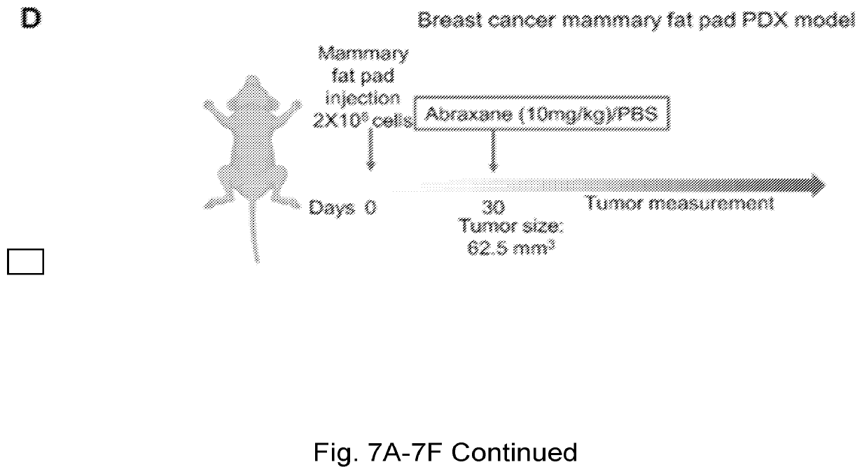

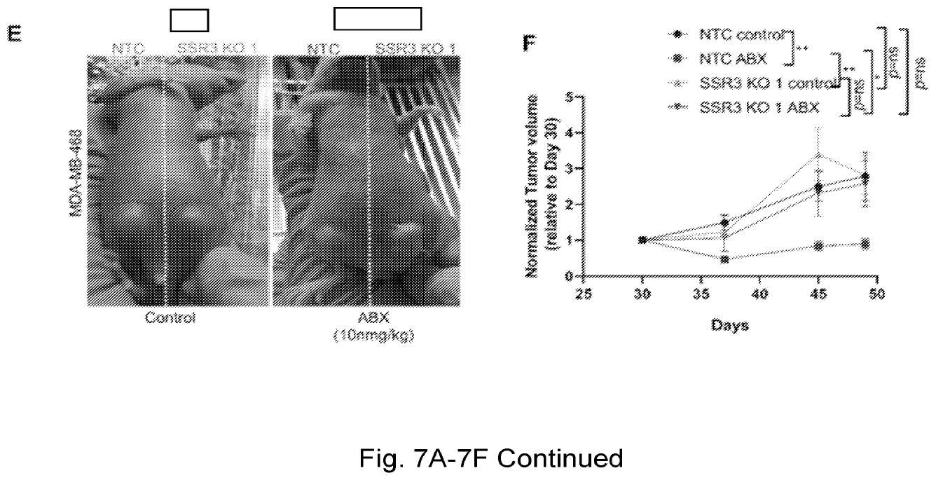

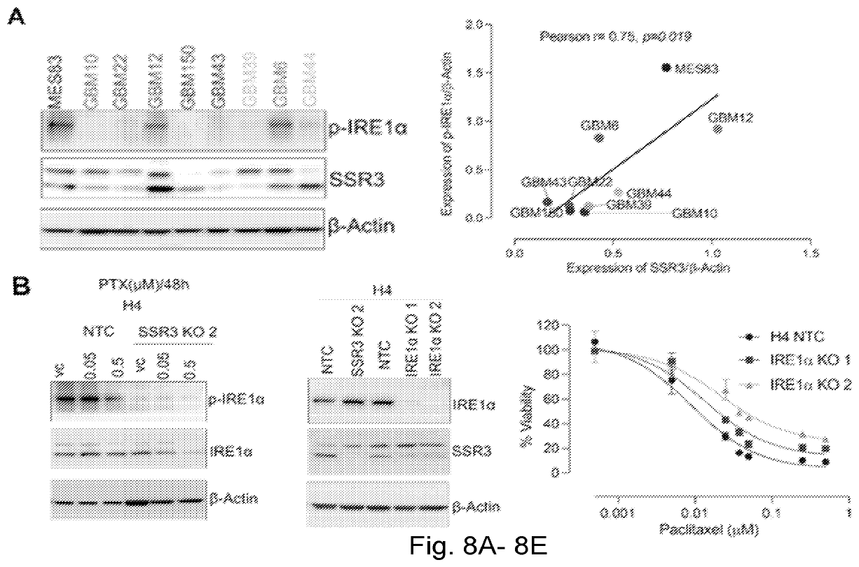

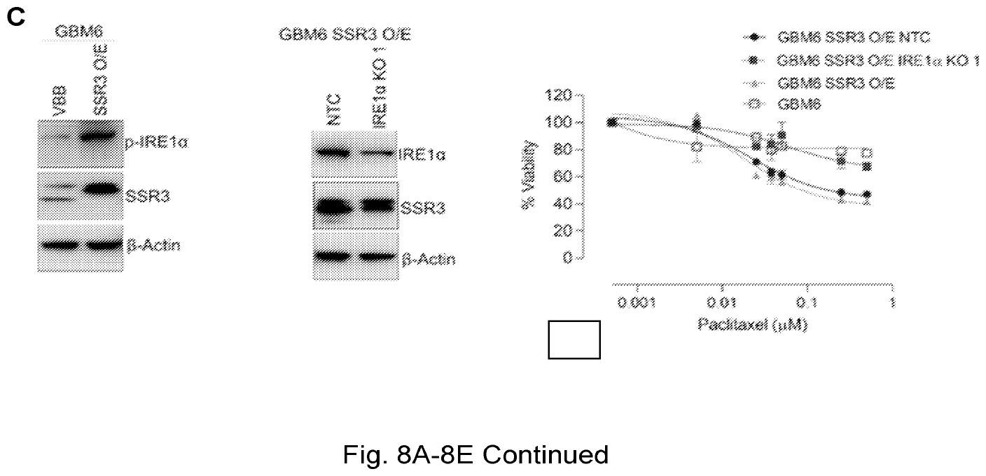

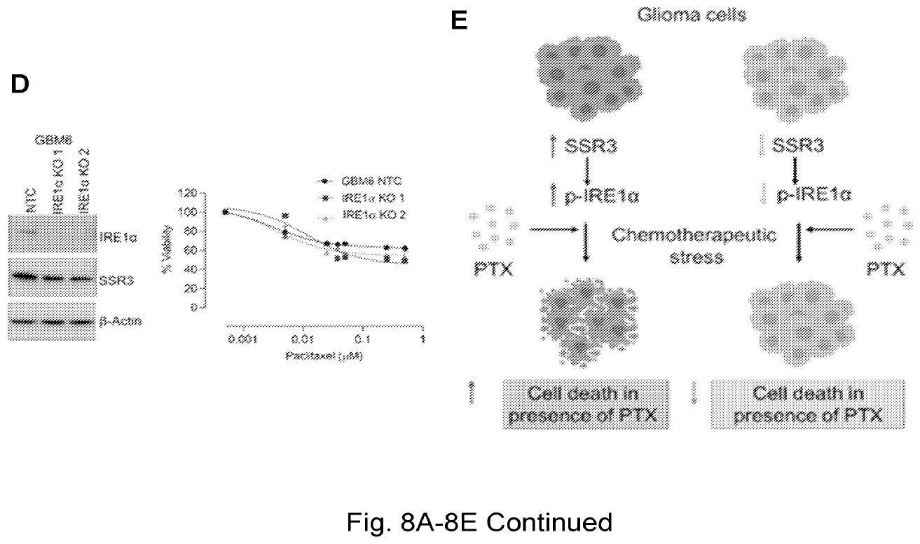

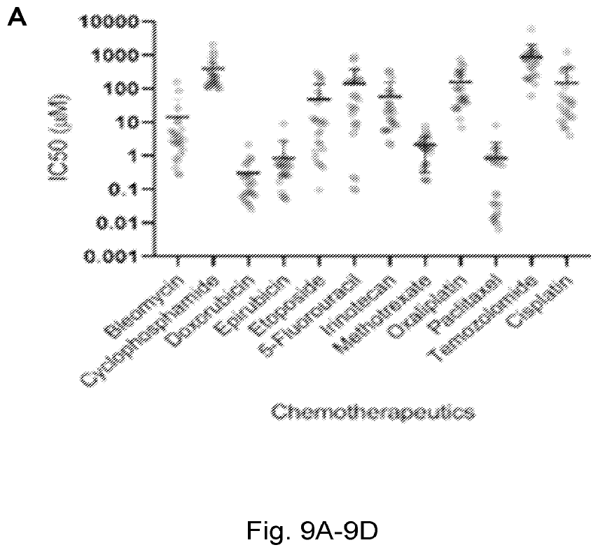

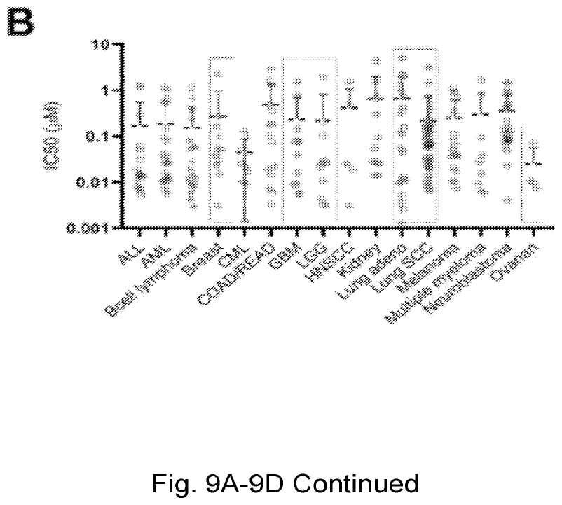

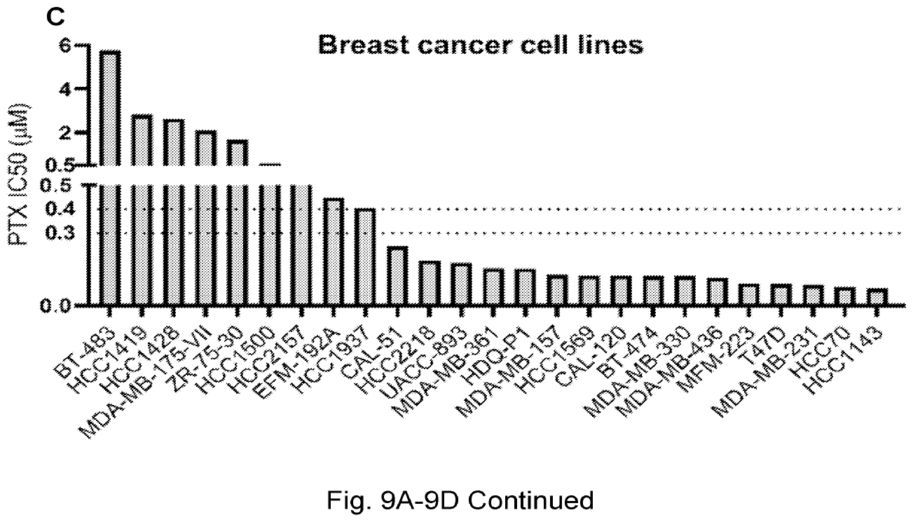

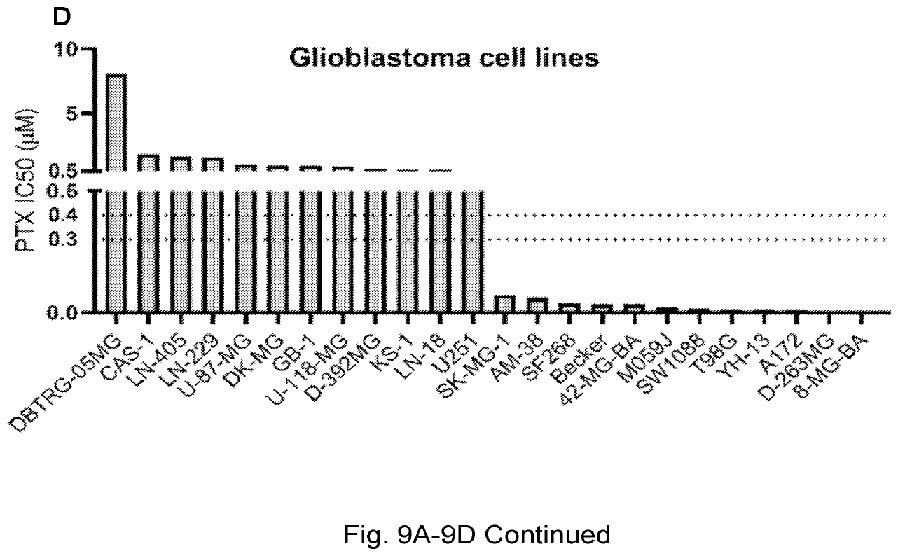

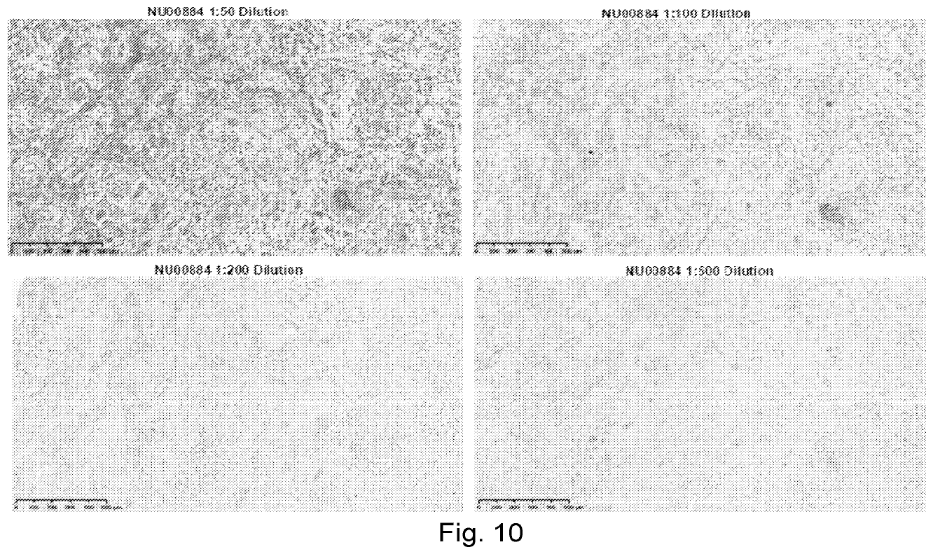

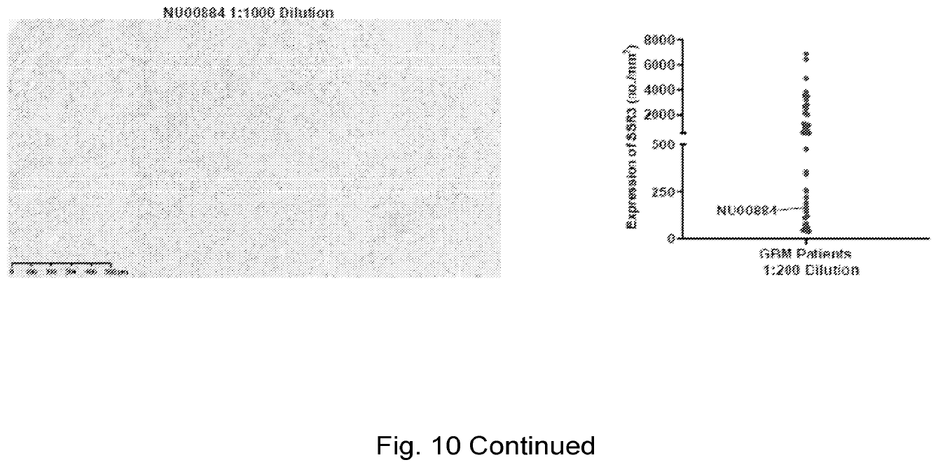

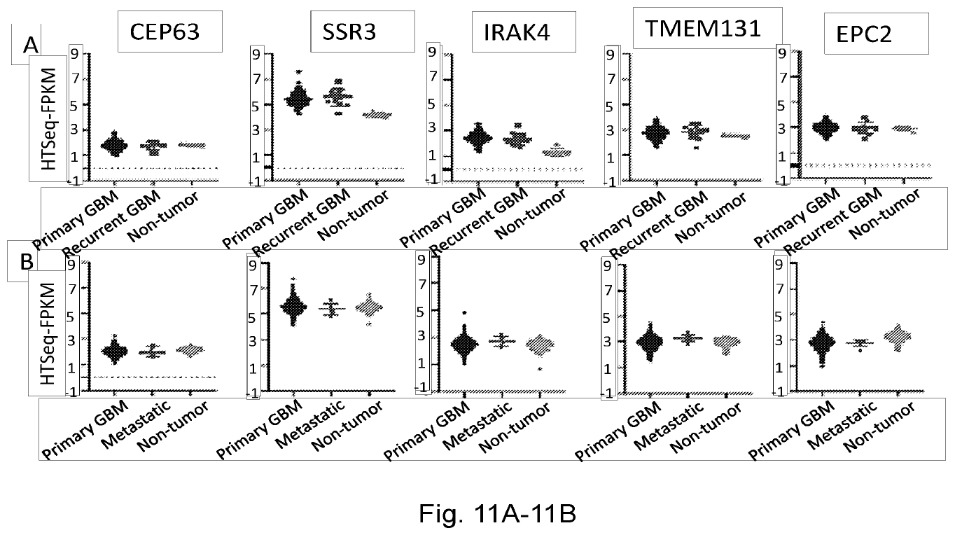

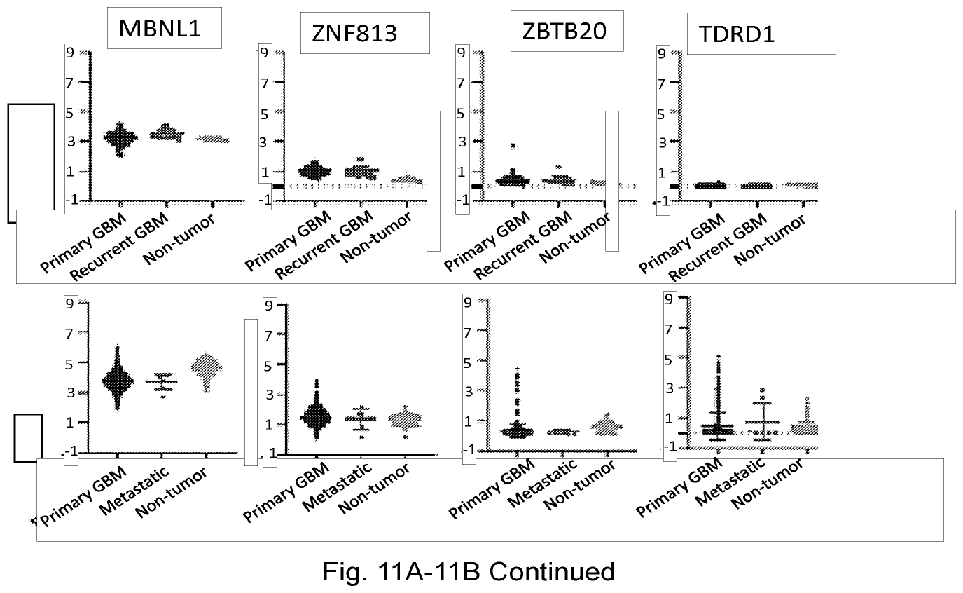

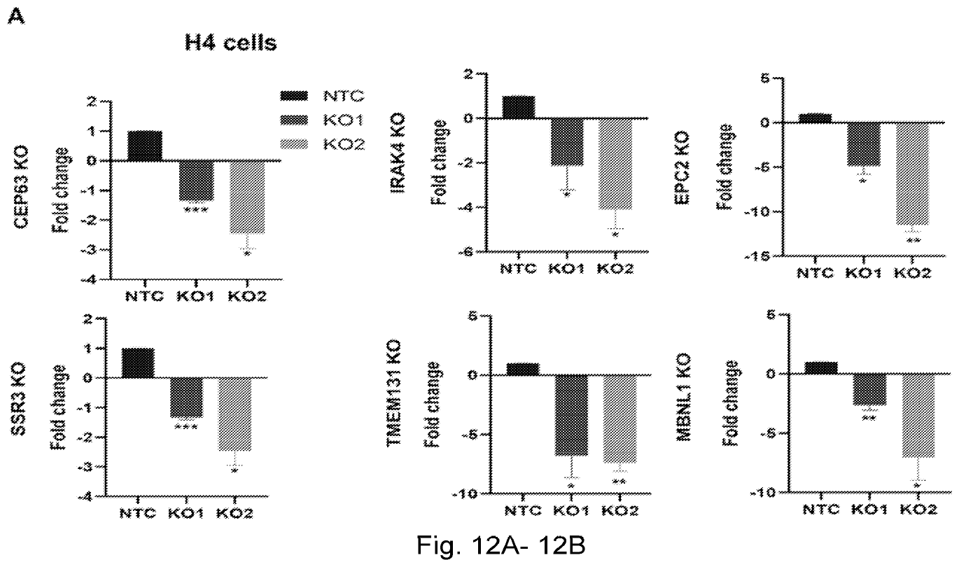

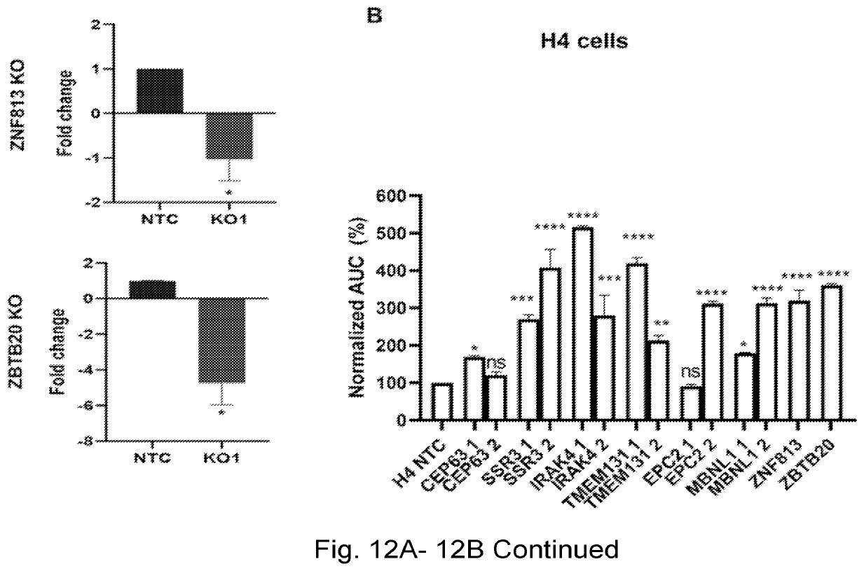

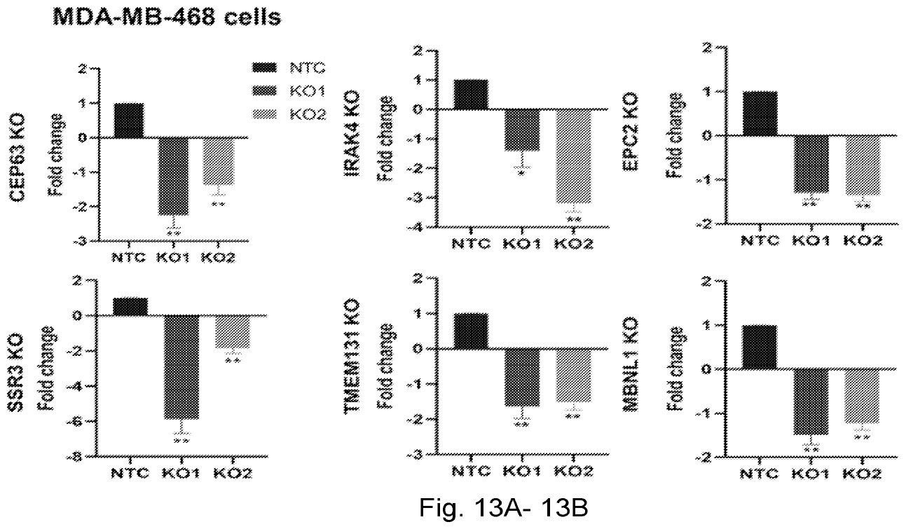

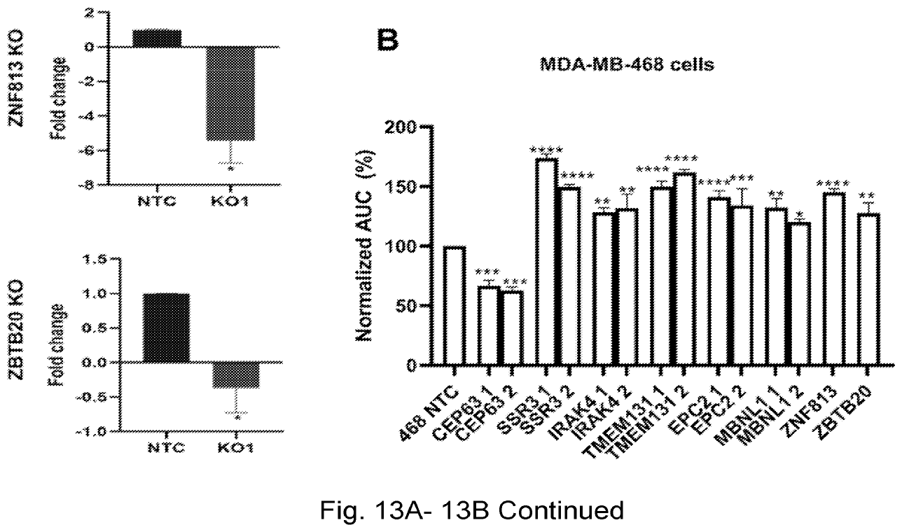

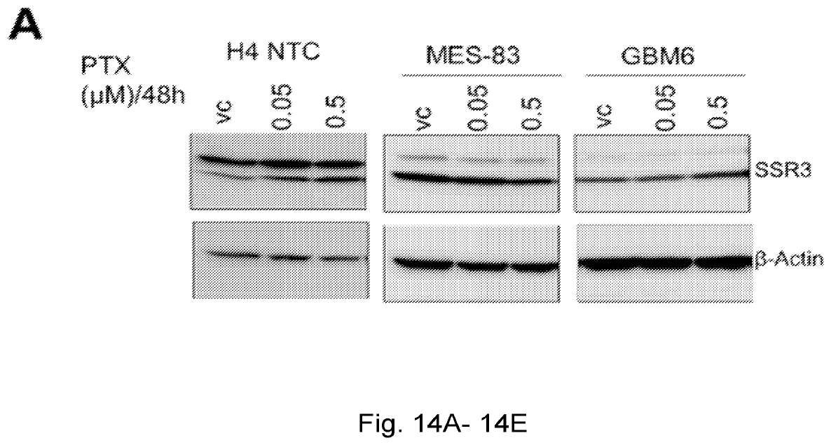

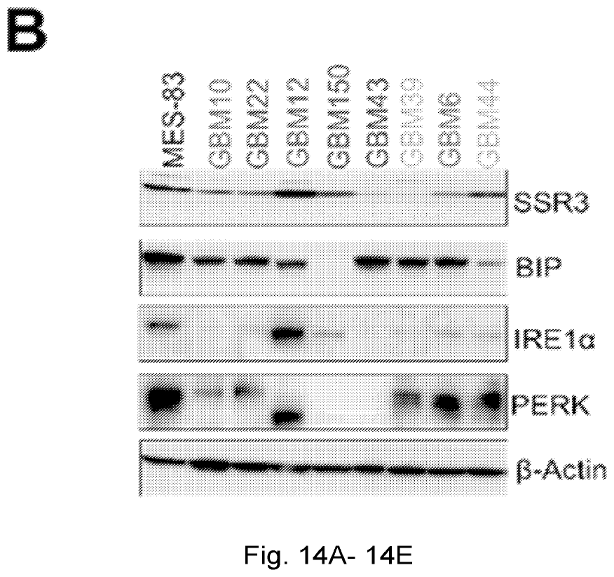

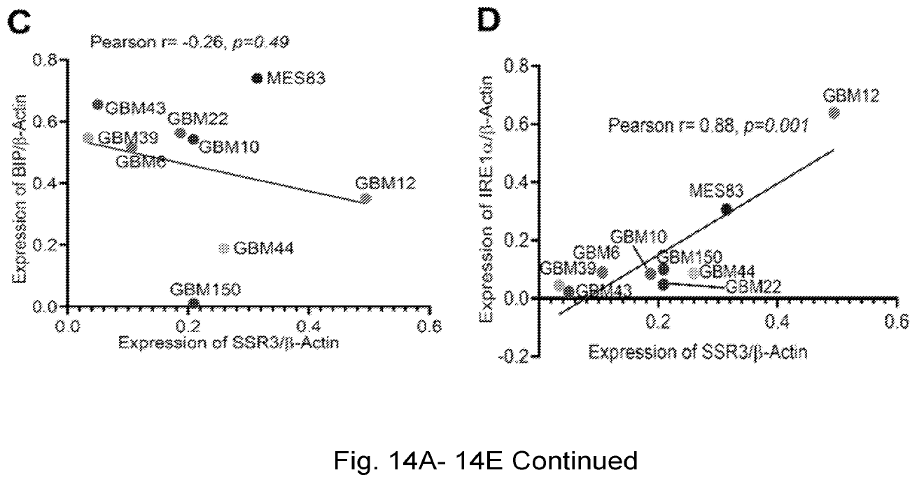

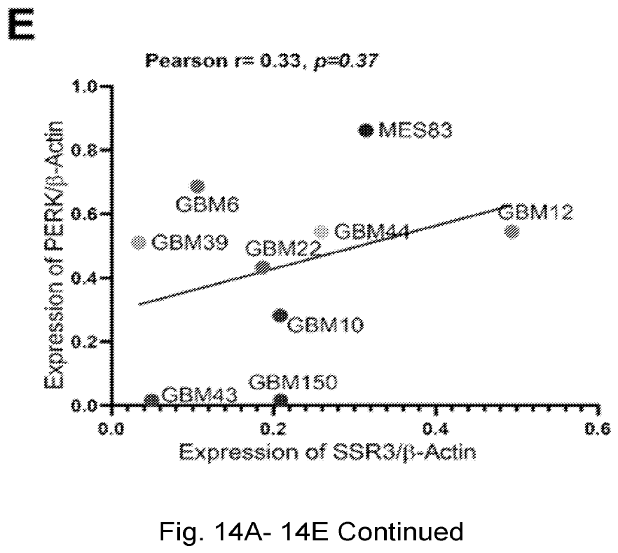

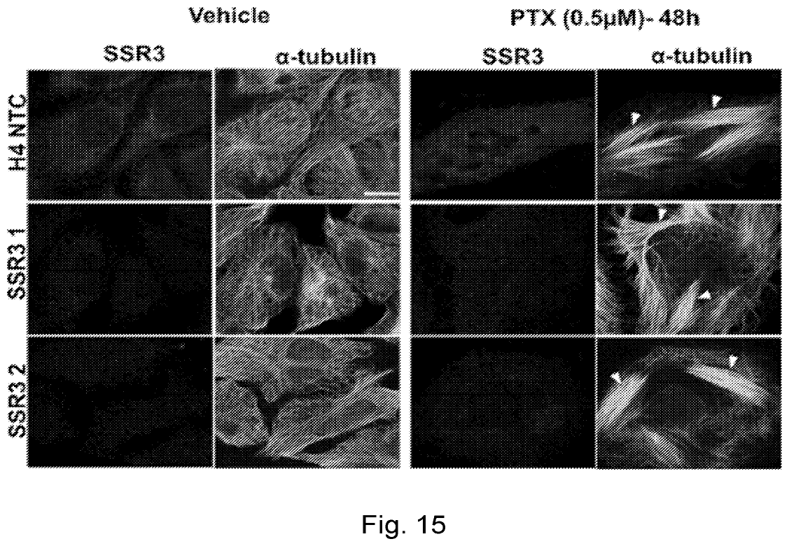

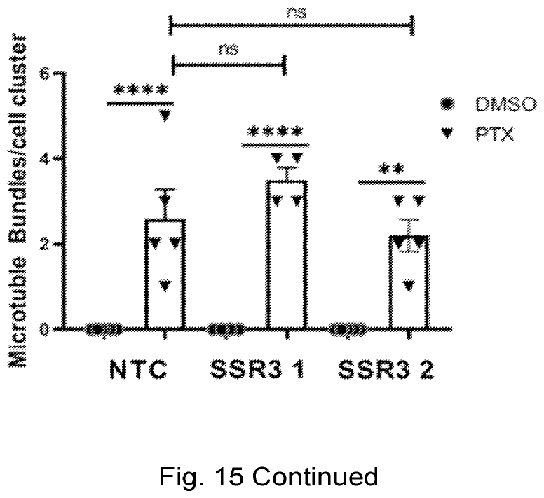

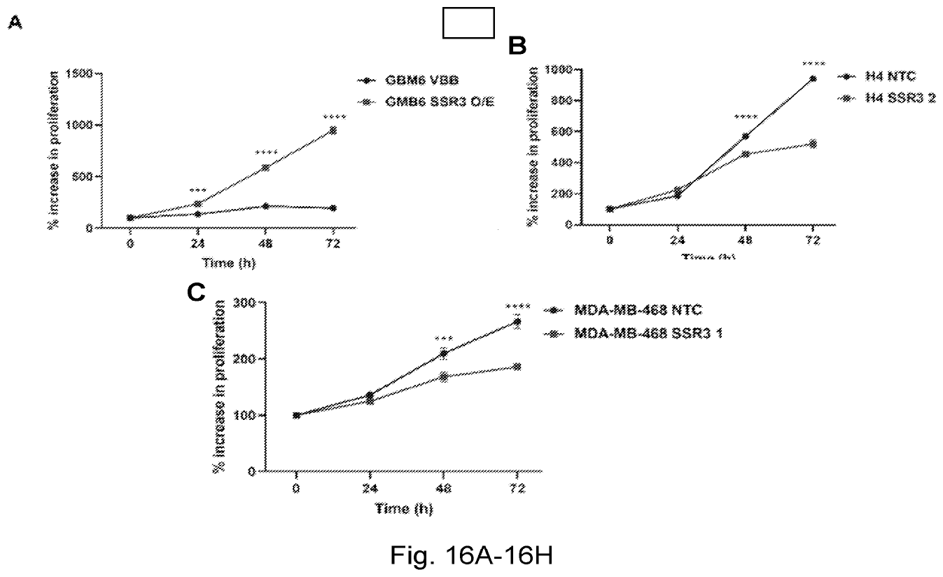

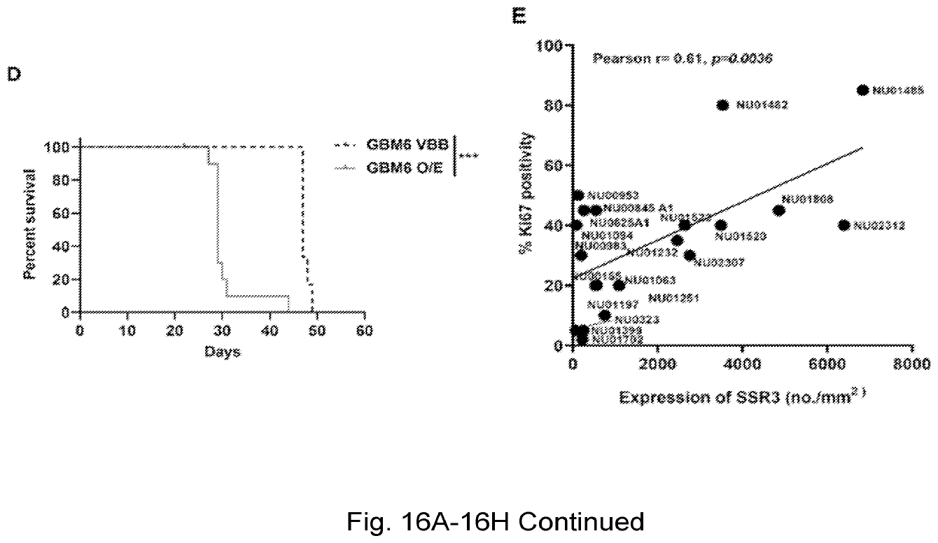

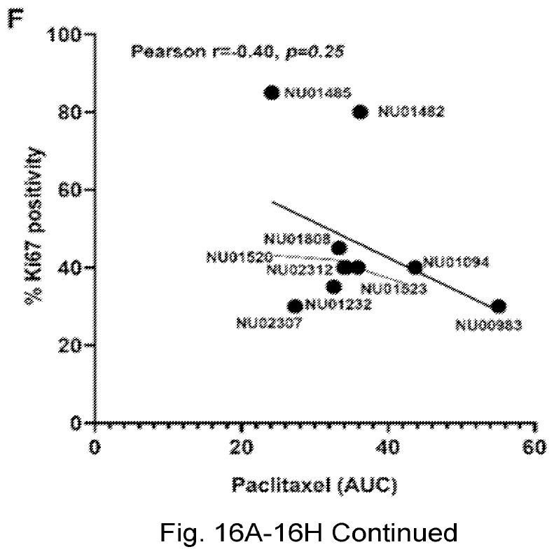

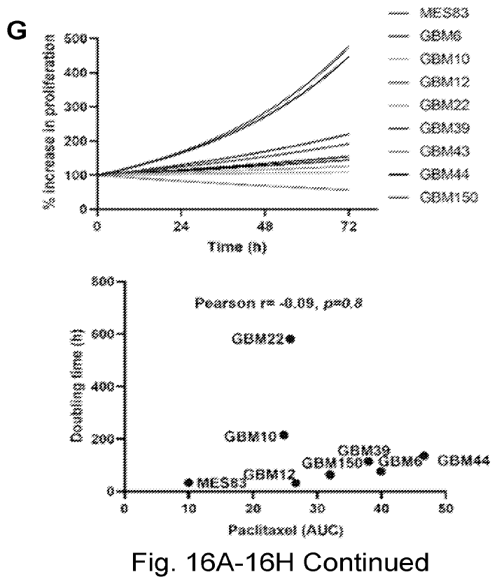

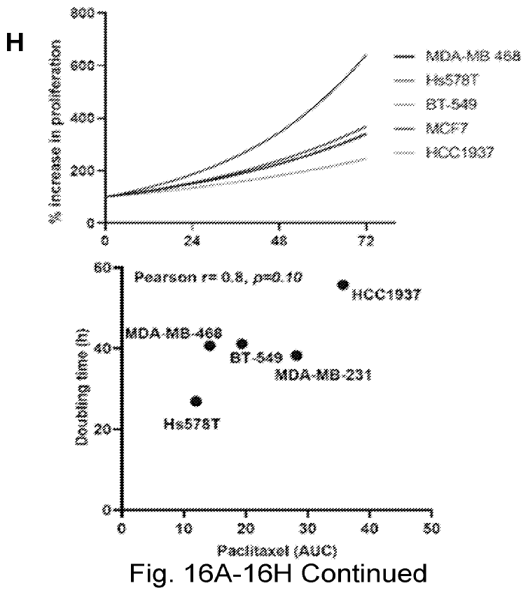

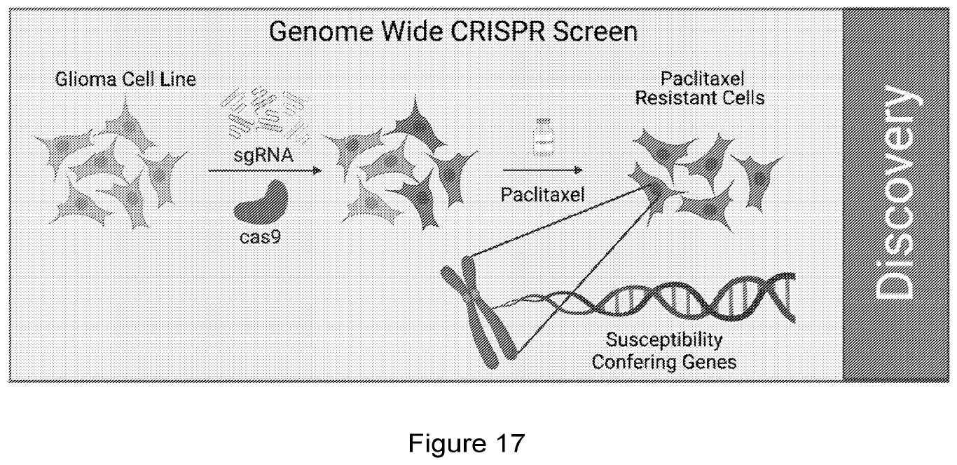

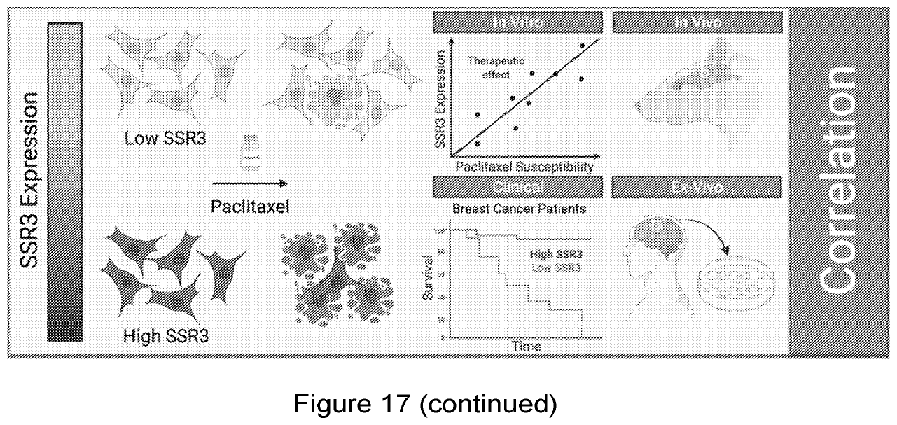

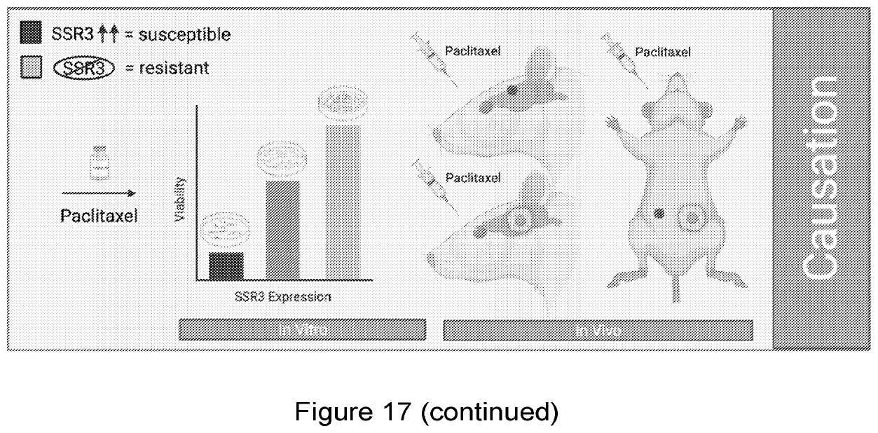

FIG. 16A-16H. S8. Proliferation advantage conferred by SSR3 may not contribute to its role in conferring susceptibility to PTX. Graph showing percent increase in proliferation in (A) vector control (GBM6 VBB) and GBM6 SSR3 overexpressing (GBM6 SSR3 O/E) clones, (B) H4 NTC and SSR3 KO 2 clones and (C) MDA-MB-468 NTC and SSR3 KO 1 clones. ***p<0.001. (D) The survival curve shows the survival of the mice injected with vector control (GBM6 VBB) and GBM6 SSR3 overexpressing (GBM6 SSR3 O/E) clones-without any treatment. The curves are adopted from FIGS. 7B and C. (E) Scatter plot showing correlation between % of Ki67 positivity (as determined by pathologist) and SSR3 staining intensity of the tumor tissue (by quantitative IHC measurement). p values, two-tailed t test. Each dot represents an independent patient sample. (F) Scatter plot showing correlation between % of Ki67 positivity (as determined by pathologist) of the tumor tissue and susceptibility to PTX (AUC) of the paired explant culture. Data is plotted for patients whose both Ki67 and explant culture was available. p values, two-tailed t test. Each dot represents an independent patient sample. (G) and (H) Graph showing percent increase in proliferation for glioma PDX cell lines and breast cancer cell lines respectively. Scatter plot showing correlation between doudling time and susceptibility to PTX (AUC). p values, two-tailed t test. Each dot represents an independent cell line.

FIG. 17. Process summary to identify predictive biomarkers for response to PTX.

### DETAILED DESCRIPTION OF THE INVENTION

The present invention is described herein using several definitions, as set forth below and throughout the application.

As used in this specification and the claims, the singular forms “a,” “an,” and “the” include plural forms unless the context clearly dictates otherwise. For example, the term “a polypeptide fragment” should be interpreted to mean “one or more a polypeptide fragment” unless the context clearly dictates otherwise. As used herein, the term “plurality” means “two or more.”

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

In some embodiments, a subject may be suffering from or diagnosed with cancer. By way of example but not by way of limitation, cancers include breast, lung, ovarian, Kaposi's sarcoma, and glioma. Gliomas comprise astrocytomas, ependymomas, glioblastomas and oligodendroglioma. In some embodiments, a cancer relevant to the present disclosure includes a cancer in which tumor tissue expression level of one or more of SSR3, IRAK4, TMEM131, EPC2, MBNL1, ZNF813, and ZBTB20 is above a control or baseline level. The present methods are not intended to be limited by cancer type. Indeed, given the high potency of taxanes against wide range of cancer types, this biomarker list can potentially be relevant for any cancer type in determining an indication for chemotherapy using taxanes.

As used herein a “subject sample” or a “biological sample” from the subject refers to a sample taken from the subject, such as, but not limited to a tissue sample (e.g, fat, muscle, skin, neurological, tumor, etc.) or fluid sample (e.g., saliva, blood, serum, plasma, urine, stool, cerebrospinal fluid, etc.), and or cells or sub-cellular structures such as vesicles and exosomes.

As used herein, the term “tubulin inhibitor” refers to a class of chemotherapeutic drugs that act by interacting with the tubulin system, e.g., by inhibiting microtubule assembly/polymerization. Non-limiting examples of such drugs include taxane, paclitaxel, docetaxel, abraxane, and taxotere, or vinca alkaloids, such as vinorelbine, vinblastine, vincristine, vindesine. Other exemplary, non-limiting tubulin inhibitors include-epothilone, discodermolide, dictyostatin, laulimalide, peloruside, maytansinoid, rhizoxin, dolastatin 10, dolastatin 15, spongistatin, halichondrin B, phomopsin, cryptophycin, colchicine, combretastatin, podophyllotoxin, chalcone, 2ME, noscapine.

### Polynucleotides

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “promoter” refers to a cis-acting DNA sequence that directs RNA polymerase and other trans-acting transcription factors to initiate RNA transcription from the DNA template that includes the cis-acting DNA sequence.

As used herein, “an engineered transcription template” or “an engineered expression template” refers to a non-naturally occurring nucleic acid that serves as substrate for transcribing at least one RNA. As used herein, “expression template” and “transcription template” have the same meaning and are used interchangeably. Engineered include nucleic acids composed of DNA or RNA. Suitable sources of DNA for use in a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms.

The polynucleotide sequences contemplated herein may be present in expression vectors. For example, the vectors may comprise a polynucleotide encoding an ORF of a protein operably linked to a promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene product.”

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

In some embodiments, therapeutic nucleic acids are employed, e.g., to decrease the level of circulating PF4 in a subject in need thereof. In some embodiments, the therapeutic nucleic acid includes one or more of an antisense oligonucleotide; DNA aptamers; gene therapy; micro RNAs; short interfering RNAs; ribozymes; RNA decoys; and circular RNAs. Two transcript (mRNA) sequences of PF4 are provided as SEQ ID NO: 3 and SEQ ID NO: 4. Therapeutic nucleic acids can be made by methods well known in the art.

### Polypeptides

The terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The amino acid sequences contemplated herein may include one or more amino acid substitutions relative to a reference amino acid sequence. For example, a variant polypeptide may include non-conservative and/or conservative amino acid substitutions relative to a reference polypeptide. “Conservative amino acid substitutions” are those substitutions that are predicted to interfere least with the properties of the reference polypeptide. In other words, conservative amino acid substitutions substantially conserve the structure and the function of the reference protein. The following Table provides a list of exemplary conservative amino acid substitutions.

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally do not maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. A “variant” of a reference polypeptide sequence may include a conservative or non-conservative amino acid substitution relative to the reference polypeptide sequence.

The disclosed peptides may include an N-terminal esterification (e.g., a phosphoester modification) or a pegylation modification, for example, to enhance plasma stability (e.g. resistance to exopeptidases) and/or to reduce immunogenicity.

A “deletion” refers to a change in a reference amino acid sequence that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence.

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

A “fusion polypeptide” refers to a polypeptide comprising at the N-terminus, the C-terminus, or at both termini of its amino acid sequence a heterologous amino acid sequence, for example, a heterologous amino acid sequence (e.g., a fusion partner) that extends the half-life of the fusion polypeptide in the tissue of interest, such as serum, plasma, fatty tissue, lymph. A “variant” of a reference polypeptide sequence may include a fusion polypeptide comprising the reference polypeptide.

A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, or 500 contiguous amino acid residues of a reference polypeptide; or a fragment may comprise no more than 5, 10, 15, 20, 25, 30, 40, 50, 60, 70, 80, 90, 100, 150, 250, or 500 contiguous amino acid residues of a reference polypeptide; or a fragment may comprise a range of contiguous amino acid residues of a reference polypeptide bounded by any of these values (e.g., 40-80 contiguous amino acid residues). Fragments may be preferentially selected from certain regions of a molecule. The term “at least a fragment” encompasses the full length polypeptide. A “variant” of a reference polypeptide sequence may include a fragment of the reference polypeptide sequence.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, or at least 700 contiguous amino acid residues; or a fragment of no more than 15, 20, 30, 40, 50, 100, 150, 200, 250, 300, 350, 400, 450, 500, 550, 600, 650, or 700 amino acid residues; or over a range bounded by any of these values (e.g., a range of 500-600 amino acid residues) Such lengths are exemplary only, and it is understood that any fragment length supported by the sequences shown herein, in the tables, figures or Sequence Listing, may be used to describe a length over which percentage identity may be measured.

In some embodiments, a “variant” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

### Predictive Genes—Biomarkers

In this study we have undertaken an unbiased approach to identify biomarkers predictive of response to PTX. First we performed a whole genome CRISPR knockout screen in a PTX-susceptible glioma cell line to identify the genes that have the most contribution for susceptibility to this drug. We have then refined this list of genes by overlapping it with genes whose expression was only associated with survival in patients with breast cancer that were treated with PTX. Further this causal and correlative biomarker list consisting of 9 genes was validated in both breast cancer and glioma cell lines by determination of PTX susceptibility following knockout in breast and glioma cell lines. This approach led to the identification of SSR3 as a major contributor to PTX susceptibility, and a predictive biomarker in breast cancer and gliomas. We found that SSR3 contributes to PTX susceptibility through modulation of endoplasmic reticulum-stress response following PTX treatment.

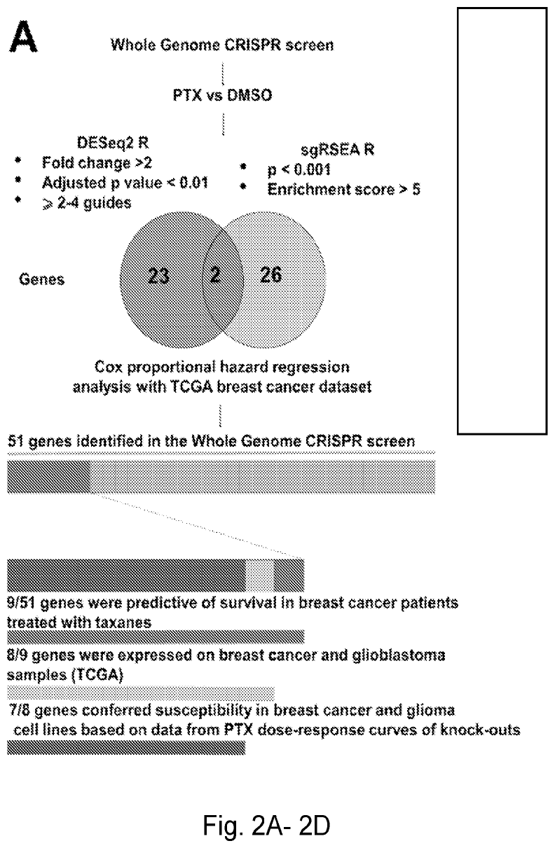

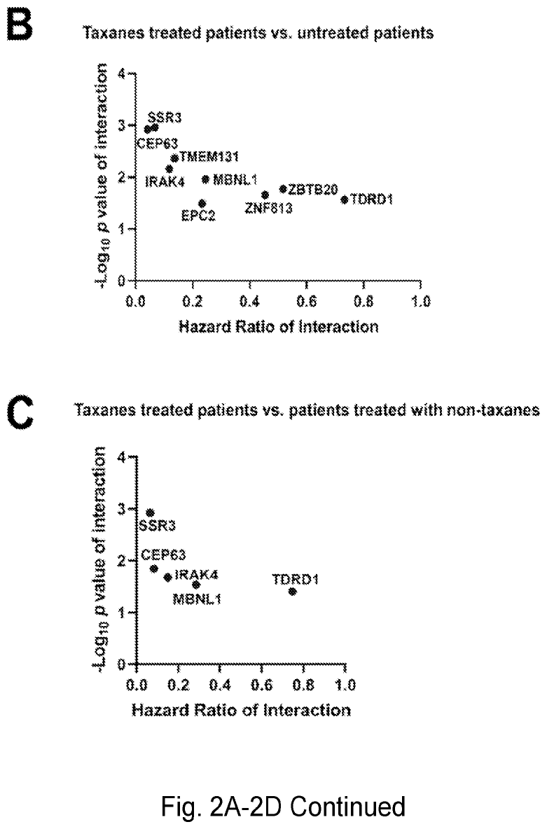

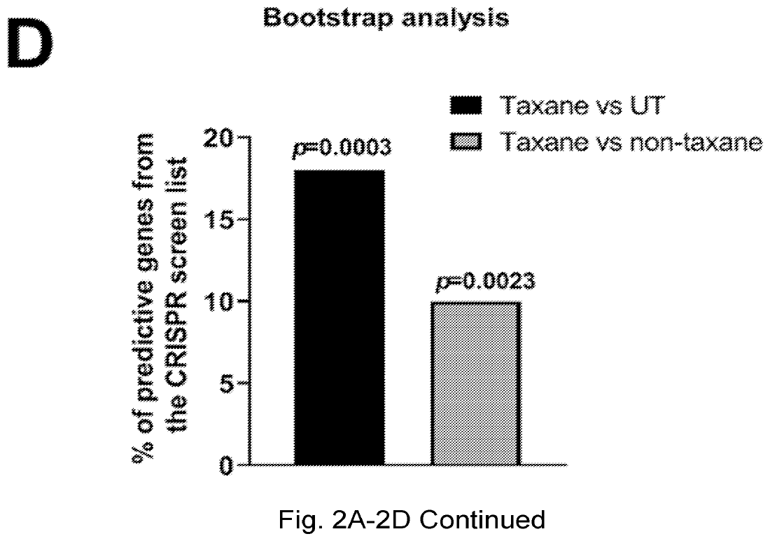

In addition to SSR3, our screen identified the following genes as predictive to PTX susceptibility, as shown in FIG. 2B: SSR3, CEP63, IRAK4, TMEM131, MBNL1, EPC2, ZNF813, ZBTB20, and TDRD1.

Among these 9 genes, SSR3, CEP63, IRAK4, MBNL1, and TDRD1 were found to be the most predictive to susceptibility in taxane-treated versus non-taxane treated samples (see e.g., FIG. 2C). Among these 5 genes, SSR3 was further analyzed.

Accordingly, the present disclosure provides methods of identifying tumors susceptible to tubulin inhibitors (e.g., taxane, paclitaxel, docetaxel, abraxane, and taxotere, or vinca alkaloids, such as vinorelbine, vinblastine, vincristine, and vindesine), in at least breast, lung, and ovarian cancer, Kaposi's sarcoma, and glioma.

The nine genes described below have multiple isoforms, and the present invention is not intended to be limited by detecting the expression of any specific isoform or set of isoforms for a given gene. For example, protein expression can be detected via a pan-antibody (i.e., an antibody that will detect all isoforms of a particular gene), or using a combination of antibodies to detect the various isoforms. Similarly, for RNA detection, primers and probes can be designed by methods well known in the art to detect the various isoforms, either individually, or collectively, in a multiplex-type reaction. Accordingly, the genes are described below and the amino acid sequence of a single isoform is provided for exemplary purposes only.

### 1. SSR3

As used herein, the term SSR3 refers to the signal sequence receptor 3 gene, also referred to as TRAPG, located on chromosome 3 (3q25.31). The signal sequence receptor (SSR) is a glycosylated endoplasmic reticulum (ER) membrane receptor associated with protein translocation across the ER membrane. The SSR is comprised of four membrane proteins/subunits: alpha, beta, gamma, and delta. The first two are glycosylated subunits and the latter two are non-glycosylated subunits. This gene encodes the gamma subunit, which is predicted to span the membrane four times. SSR3 is ubiquitously expressed in thyroid, endometrium and 25 other tissues. The 133 amino acid sequence of SSR3 isoform 1 is (SEQ ID NO: 47) (NCBI Reference Sequence: NP_001295126.1)

### 2. MBNL1

As used herein, the term “MBNL1” refers to muscleblind like splicing regulator 1, located on chromosome 3 (3q25.1-q25.2), which is an RNA splicing protein that in humans is encoded by the MBNL1 gene. It has a well characterized role in myotonic dystrophy wherein impaired splicing disrupts muscle development and function. Interestingly, in addition to regulating mRNA maturation of hundreds of genes MBNL1 (along with its paralogs MBNL2 & MBNL3) autoregulate alternative splicing of the MBNL1 pre-mRNA transcript. The founding member of the human MBNL family of proteins was the Drosophila Muscleblind protein (PMID: 9334280).

Human MBNL1 is an alternative splicing regulator that harbors dual function as both a repressor and an activator for terminal muscle differentiation. The repressive function of Human MBNL1 by sequestering at normal splice sites has been shown to lead to RNA-splicing defects that lead to muscular diseases.

Human MBNL1 (isoform 1) is a 370 amino acid protein composed of four Zinc Finger protein domains of the CCCH type linked in tandem. The MBNL1 protein specifically binds to double stranded CIG RNA expansions. The Zinc Finger domains play a role in both protein:protein contacts as well as RNA:protein contacts when bound to an oligonucleotide. There are 8 known MBNL1 isoforms produced by alternative splicing. The amino acid sequence of isoform 8, which is 343 amino acids is SEQ ID NO: 48 (NCBI Reference Sequence: NP_001300986.1).

### 3. TDRD1

As used herein, the term “TDRD1” refers the tudor domain-continain protein 1, located on chromosome 10 (10q25.3), which plays a central role during spermatogenesis by participating in the repression transposable elements and preventing their mobilization, which is essential for the germline integrity. TDRD1 acts via the piRNA metabolic process, which mediates the repression of transposable elements during meiosis by forming complexes composed of piRNAs and Piwi proteins and governs the methylation and subsequent repression of transposons. It is also required for the localization of Piwi proteins to the meiotic nuage. TDRD1 is involved in the piRNA metabolic process by ensuring the entry of correct transcripts into the normal piRNA pool and limiting the entry of cellular transcripts into the piRNA pathway. It may act by allowing the recruitment of piRNA biogenesis or loading factors that ensure the correct entry of transcripts and piRNAs into Piwi proteins. There are four TDRD1 isoforms. The amino acid sequence of TDRD1 isoform 2 is SEQ ID NO: 49 (NCBI Reference Sequence: NP_001352820.1).

### 4. CEP63

As used herein, the term “CEP63” refers to centrosomal protein of 63 kD. RCEP63, located on chromosome 3 (3q22.2) is required for normal spindle assembly and plays a key role in mother-centriole-dependent centriole duplication; the function seems also to involve CEP152, CDK5RAP2 and WDR62 through a stepwise assembled complex at the centrosome that recruits CDK2 required for centriole duplication. CEP 63 is reported to be required for centrosomal recruitment of CEP152; however, this function has been questioned. CEP63 also recruits CDK1 to centrosomes. It also plays a role in DNA damage response. Following DNA damage, such as double-strand breaks (DSBs), CEP63 is removed from centrosomes; this leads to the inactivation of spindle assembly and delay in mitotic progression. There are four CEP63 isoforms. The amino acid sequence of isoform c is SEQ ID NO: 50 (NCBI Reference Sequence: NP_001035842.1).

### 5. IRAK4

As used herein, the term “IRAK4” refers to interleukin-1 receptor-associated kinase 4. IRAK4 is a serine/threonine-protein kinase located on chromosome 12 (12q12) that plays a critical role in initiating innate immune response against foreign pathogens. IRAK4 is involved in Toll-like receptor (TLR) and IL-IR signaling pathways. It is rapidly recruited by MYD88 to the receptor-signaling complex upon TLR activation to form the Myddosome together with IRAK2 IRAK4 phosphorylates initially IRAK1, thus stimulating the kinase activity and intensive autophosphorylation of IRAK1. It also phosphorylates E3 ubiquitin ligases Pellino proteins (PELI1, PELI2 and PELI3) to promote pellino-mediated polyubiquitination of IRAK1. Then, the ubiquitin-binding domain of IKBKG/NEMO binds to polyubiquitinated IRAK1 bringing together the IRAK1-MAP3K7/TAK1-TRAF6 complex and the NEMO-IKKA-IKKB complex. In turn, MAP3K7/TAK1 activates IKKs (CHUK/IKKA and IKBKB/IKKB) leading to NF-kappa-B nuclear translocation and activation. Alternatively, phosphorylates TIRAP to promote its ubiquitination and subsequent degradation. IRAK4 also phosphorylates NCF1 and regulates NADPH oxidase activation after LPS stimulation suggesting a similar mechanism during microbial infections. Two isoforms have been identified, and the amino acid sequence of isoform a is SEQ ID NO: 51 (NCBI Reference Sequence. NP_001107654.1).

### 6. TMEM131

As used herein, the term “TMEM131” refers to transmembrane protein 131, and is located on chromosome 2 (2q11.2). The TMEM131 protein contains three domains of unknown function 3651 (DUF3651) and two transmembrane domains. This protein has been implicated as having a role in t-cell function and development. MEM131 also resides in a locus (2q11.1) that is associated with Nievergelt's syndrome when deleted. TMEM131 has been shown to exhibit hypermethylation in patients with Down syndrome. The authors of this study proposed that, given TMEM131 is supposed function in T cell development and function, this hypermethylation may play a role in the suppressed immune function in patients with Down syndrome. TMEM131 has also been shown to be up-regulated during the development and differentiation of T cells, and has been shown to have relatively high levels of expression in T cells relative to other tissue types.

TMEM131 is expressed in low to moderate levels throughout most of the body, with slightly increased levels occurring in the lymph nodes, uterus, and T cells. The amino acid sequence of TMEM131 is SEQ ID NO: 52 (NCBI Reference Sequence: NP_056163.1).

### 7. EPC2 Gene

As used herein, the term “EPC2” refers to the human homologue of enhancer of polycomb homolog 2 (from Drosophila), located on human chromosome 2 (2q23.1). EPC2 is expressed in numerous tissues and organs, with higher expression levels in the endometrium, brain, lymph node, ovary, testis, urinary bladder, and thyroid. The amino acid sequence of human EPC2 is SEQ ID NO: 53 (NCBI Reference Sequence: NP_056445.3).

### 8. ZNF813

As used herein, the term “ZNF813” refers to the zinc finger protein 813, located on chromosome 19 (19q13.42). ZNF813 is expressed is expressed in numerous tissues and organs, with higher expression levels in placenta and prostate. The amino acid sequence of ZNF813 is SEQ ID NO: 54.

### 9. ZBTB20

As used herein, the term “ZBTB20” refers to the zinc finger and BTB domain containing 20 gene, and is located on chromosome 3 (3q13.31). This gene, which was initially designated as dendritic cell-derived BTB/POZ zinc finger (DPZF), belongs to a family of transcription factors with an N-terminal BTB/POZ domain and a C-terminal DNA-bindng zinc finger domain. The BTB/POZ domain is a hydrophobic region of approximately 120 aa which mediates association with other BTB/POZ domain-containing proteins. This gene acts as a transcriptional repressor and plays a role in many processes including neurogenesis, glucose homeostasis, and postnatal growth. Mutations in this gene have been associated with Primrose syndrome as well as the 3q13.31 microdeletion syndrome. Alternative splicing results in multiple transcript variants encoding distinct isoforms. ZBTB20 is ubiquitously expressed in the kidney, ovary, and 25 other tissues. The amino acid sequence of ZBTB20 isoform 1 is SEQ ID NO: 55 (NCBI Reference Sequence: NP_001157814.1).

### Methods of Detecting Biomarkers

As used herein, the term “biomarker” refers to a characteristic that is objectively measured and evaluated as an indicator of normal biological processes, pathogenic processes, or pharmacological responses to a therapeutic intervention. By way of example but not by way of limitation, biomarkers disclosed herein include products of gene expression such as RNA and/or protein. In some embodiments, an aberrant level of gene expression (e.g., an increased or decreased level of expression in a tumor sample from a subject suffering from cancer) as compared to a control, threshold, or baseline level of expression is indicative of susceptibility or resistance of the tumor to one or more therapeutic treatments.

Exemplary genes that serve as biomarkers for susceptibility or resistance to cancer therapeutics, such as tubulin inhibitors, includes seven of those described above (e.g., SSR3, IRAK4, TMEM131, EPC2, MBNL1, ZNF813, and ZBTB20; SEQ ID NOs: 47-55, or an RNA sequence encoding one of SEQ ID NOs: 47-55). In some embodiments, an increased level of expression of one or more of these biomarker in a tumor sample from a subject indicates susceptibility to a cancer therapy such as a tubulin inhibitor (e.g., taxane, paclitaxel, docetaxel, abraxane, and taxotere, or vinca alkaloids, such as vinorelbine, vinblastine, vincristine, and vindesine).

A subject's tumor biomarker level (e.g., the level of expression of one or more of SSR3, IRAK4, TMEM131EPC2, MBNL1, ZNF813, and ZBTB20 SEQ ID NOs: 47-55, or an RNA sequence encoding one of SEQ ID NOs: 47-55) can be assessed by evaluating a tumor biopsy sample, including for example, tissue, cells, blood, bone, neurological tissues or cells, etc. taken from the subject. Control, threshold, or baseline levels for a given biomarker may be determined from tumor tissue or matched wild-type or non-tumor tissue from the same subject, different subjects, or from cohort of subjects. In some embodiments, a control, threshold, or baseline level may be “0” or negligible expression. Thus in some embodiments, any level of expression is indicative of better or more favorably therapeutic efficacy of a tubulin inhibitor such as paclitaxel as compared to a sample in which no or negligible expression is detected.

Thus, by determining a subject's biomarker expression level in a tumor sample and comparing the expression level to a control, threshold, or baseline level of the same biomarker, a determination can be made regarding the tumor's resistance or susceptibility to one or more chemotherapeutic drugs, or to a class of chemotherapeutic drugs (e.g., tubulin inhibitors). This information can then be used, combined with clinical data (such as sex, age, height, weight, prior medical history, prior and current treatments or therapies, molecular/genetic data, tumor type, tumor stage, etc.) to assist physicians in more accurately directing treatment.

Methods to determine the protein level of one or more biomarkers (e.g., SSR3, IRAK4, TMEM131EPC2, MBNL1, ZNF813, and ZBTB20, e.g., SEQ ID NOs: 47-55) include, but are not limited to: immunoassays assays, such as ELISA and Western blotting; chromatographic methods; and protein mass spectrometry assays. Antibodies that bind to specific proteins are well-known in the art and some are commercially available, as are ELISA kits.

Additionally or alternatively, biomarker levels (e.g., SSR3, IRAK4, TMEM131EPC2, MBNL1, ZNF813, and ZBTB20), can be determined by evaluating a subject's biomarker RNA (mRNA) levels, e.g., an RNA sequence encoding one of SEQ ID NOs: 47-55. Methods to detect RNA are well known in the art, and numerous kits and options are commercially available. By way of example, but not by way of limitation, methods include reverse transcription and polymerase chain reaction, (RT-PCR), methods employing direct oligonucleotide probe hybridization to the biomarker RNA e.g., Northern blotting.

As used herein, the term control sample, control level, or control subject, refer to a sample, level, or subject that is considered “normal” or “wild-type” relative to the specific condition or conditions under investigation. In some embodiments, a biomarker control or baseline level is the level of the biomarker identified in a subject or a cohort of subjects (e.g., pooled samples, or averaged values) that are typically not responsive to (e.g., exhibit no or negligible positive therapeutic response to) a particular therapeutic, such as a tubulin inhibitor. In some embodiments, the control samples comprise the same tissue as the subject's tumor tissue. In some embodiments, a “control” level is determined by comparing the level of expression of a particular biomarker in several tumor samples of the same type from a cohort of different subjects. The levels are compared and a threshold level or a baseline level is determined: expression levels below the threshold or baseline level are considered “low” and are therefore indicative of resistance to a therapeutic drug; expression levels above the threshold or baseline level are considered “elevated” and are therefore indicative of susceptibility to a therapeutic drug.

In some embodiments, a range of values is used to determine response to a particular therapeutic. For example, the selection of the nine biomarkers described above was done by correlation analysis of expression of these genes with chemotherapy drug used (taxanes, other drugs, or no chemotherapy) with overall survival in a continuous fashion. Accordingly, in some embodiments, every unit increase in the expression of one or more of these biomarkers is correlated to a corresponding increase in survival of a patient treated with a tubulin inhibitor such as paclitaxel. Therefore the present disclosure encompasses an incremental trend in baseline expression of one or more of these nine biomarkers as predictive of response to a tubulin inhibitor such as paclitaxel in a continuous fashion.

As described herein, an elevated level of RNA or protein of one or more biomarkers of the present disclosure (e.g., SSR3, IRAK4, TMEM131, EPC2, MBNL1, ZNF813, and ZBTB20) in a tumor sample is indicative of susceptibility to treatment with a chemotherapeutic agent such as a tubulin inhibitor. In some embodiments, an elevated biomarker level is characterized as an incremental trend in baseline expression of one or more of the nine biomarkers as predictive of response to paclitaxel in a continuous fashion while a degressive trend in baseline expression of one or more of these nine biomarkers corresponds to resistance to tubulin inhibitor (e.g., paclitaxel) chemotherapy.

In some embodiments, a subject's tumor biomarker levels can be determined before, during, and/or after a course of treatment or therapy, or throughout the subject's life, e.g., if a genetic predisposition exists or if clinical symptoms regularly appear. In some embodiments, a subject's tumor biomarker level is evaluated two or more times, for example, over the course of a week, a month, three to six months, or a year or more.

In some embodiments, after a subject's tumor has been identified as having elevated biomarker levels, the subject may be subject to further diagnostic methods and/or treatment methods for the cancer, e.g., surgery, radiation, and chemotherapy in addition to tubulin inhibitors.

### Methods of Treatment

### Pharmaceutical Compositions and Methods of Treatment

Tubulin inhibitors such as paclitaxel and taxane are among the most commonly used group of chemotherapeutics used for cancer as these drugs are indicated for ovarian, breast, and lung cancer, as well as Kaposi's sarcoma, and are also used off-label for many other scenarios in oncology. Given the limited efficacy of current therapies for glioblastoma (GMB), novel therapies are desperately needed. While the blood-brain barrier (BBB) prohibits conventional administration of tubulin inhibitors to treat GMB, emerging efforts have led to the effective delivery of some drugs across the BBB. Accordingly, determining the susceptibility of a subject's tumor tissue to a chemotherapeutic agent such as a tubulin inhibitor allows for more effective treatment-resulting in increased survival time, tumor regression, or complete or partial remission—for subjects suffering from various forms of cancer.

Therapeutic compositions disclosed herein include tubulin inhibitors, such as taxane, paclitaxel, docetaxel, abraxane, and taxotere, or vinca alkaloids, such as vinorelbine, vinblastine, vincristine, and vindesine. Such compositions can be formulated and/or administered in dosages and by techniques well known to those skilled in the medical arts taking into consideration such factors as the age, sex, weight, tumor type and stage, condition of the particular patient, and the route of administration.

The compositions may include pharmaceutical solutions comprising carriers, diluents, excipients, preservatives, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”).

In some embodiments, compositions are formulated for systemic delivery, such as oral or parenteral delivery. In some embodiments, minimally invasive microneedles and/or iontophoresis may be used to administer the composition. In some embodiments, compositions are formulated for site-specific administration, such as by injection into a specific tissue or organ, topical administration (e.g., by patch applied to the target tissue or target organ). In some embodiments, the composition is formulated to be delivered through the BBB. By way of example, but not by way of limitation, such methods may include ultrasound treatment with or without concomitant administration of microbubbles, convection enhanced drug delivery, biodegradable wafers that release the drug, peptide-drug conjugates, and nanoparticle-drug coupling to enhance drug penetration across the BBB.

The therapeutic composition may include, in addition to tubulin inhibitor, one or more additional active agents. By way of example, the one or more active agents may include an additional chemotherapeutic drug, an antibiotic, anti-inflammatory agent, a steroid, or a non-steroidal anti-inflammatory drug.

According to various aspects, tubulin inhibitor, and optionally the one or more active or inactive agents may be present in the composition as particles or may be soluble. By way of example, in some embodiments, micro particles or microspheres may be employed, and/or nanoparticles may also be employed, e.g., by utilizing biodegradable polymers and lipids to form liposomes, dendrimers, micelles, or nanowafers as carriers for targeted delivery of the tubulin inhibitors. In some embodiments, polymeric implants may be used. By way of example, but not by way of limitation, in some embodiments, a therapeutic composition comprising a tubulin inhibitor is applied to a patch and placed in contact with the target tissue.

In some embodiments, the composition formulated for administration comprises between 500 mg/ml and 1000 mg/ml of the tubulin inhibitor. In some embodiments, the composition formulated for administration comprises between 0.1 ng and 500 mg/ml of the inhibitor. In some embodiments, the compositions if formulated such that between 0.1 ng and 500 μg/ml of the inhibitor is administered to a subject. In some embodiments, the composition is administered at between 500 mg/ml and 1000 mg/ml of inhibitor; between 0.1 ng and 500 mg/ml of the inhibitor; or between about 0.1 ng and 500 μg/ml of the inhibitor. In some embodiments, at least about 0.1-1.0 μM is administered to a subject. In some embodiments, at least about 0.2-0.7 μM, or 0.3-0.5 μM is administered to a subject.

In some embodiments, the methods include administration of the therapeutic compositions once per day; in some embodiments, the composition may be administered multiple times per day, e.g., at a frequency of one or two times per day, or at a frequency of three or four times per day or more. In some embodiments, the methods include administration of the composition once per week, once per month, or as symptoms dictate.

In some embodiments, in addition to one or more therapeutic formulations, a subject is also administered an additional cancer treatment, such as surgery, radiation, immunotherapy, stem cell therapy, and hormone therapy.

In some embodiments, a subject tumor sample with a biomarker expression level higher than a control or a baseline level is treated with one or more tubulin inhibitors. In some embodiments, the treatment reduces, alleviates, prevents, or otherwise lessens the symptoms of the tumor more quickly or effectively than a subject suffering the same or similar cancer, but with a tumor biomarker level at or below the control or threshold level.

In some embodiments, improvements in the condition of the subject's cancer status and overall health is observed more quickly than if no treatment is provided for the same or similar condition or disease.

### Implementations and Advantages

Paclitaxel (PTX) is a commonly used drug in the treatment of breast, lung and ovarian cancers. This drug is exquisitely potent against gliomas and in particular glioblastoma, with an IC50 approximately 1400-fold lower than that of temozolomide, the standard of care for these tumors. Several clinical trials explore means of enhancing delivery of PTX across the blood-brain barrier for glioblastomas. In our upcoming clinical trial we are repurposing PTX to treat glioblastomas (GBMs) using ultrasound mediated delivery of this drug in the human brain.

In spite of the efficacy of PTX, individual tumors exhibit variable and unpredictable susceptibility to this drug, with a response rate of 30%-60%, and there is no clinically available biomarker to identify susceptible tumors. To identify predictive biomarkers to personalize the use of PTX, we performed a genome-wide CRISPR knock-out screen. The CRISPR screen led to identification of 51 genes, knocking out these genes rendered cells resistant to PTX. These 51 genes were then tested for their ability to predict survival of the TCGA breast cancer patients treated with PTX, but not of breast cancer patients that did not received PTX (Marginal structural Cox model, MSCM). 9/51 (18%) genes that were found to be predictive of PTX response, were validated by single gene KO experiments in glioma and breast cancer cell lines. The SSR3 gene was found to be the best performing biomarker in conferring susceptibility as well as in predicting response to PTX treatment in both glioma and breast cancer models. SSR3 levels correlated with response to PTX in breast cancer and glioma cells, across multiple intracranial glioma xenografts and in human glioblastoma specimens that were paired with explant cultures. SSR3 sensitizes tumor cells to PTX by dampening the pro-survival ER stress response. SSR3 is an endoplasmic reticulum (ER) resident protein which plays crucial role in ER transport and ER stress response. Most of the ER stress regulators were depleted in the screen implying their role in PTX resistance. Without wishing to be bound by theory, SSR3 seems to dampen cancer cell's pro-survival ER stress response to resist PTX induced cytotoxicity, as a result conferring susceptibility.

Implementation of these biomarkers for clinical decision making will allow maximum benefit for the responder patient cohort while spare the non-responder cohort from undergoing the treatment without any benefit. The non-responder patient cohort identified by the biomarker panel can be offered alternative treatment.

This biomarker panel can be used to identify taxane (paclitaxel/docetaxel/paclitaxel formulation/s) responders among wide patient population including breast cancer, breast cancer brain metastasis and glioblastoma patients.

Upon further validation these biomarkers can be extrapolated to identify responder patients in other cancers commonly treated with taxanes like ovarian, lung, cervical, prostate and pancreatic cancers.

Our findings also suggest the potential of these biomarkers to predict response to other microtubule inhibitors including but not limited to vinorelbine.

Taxanes are very potent and very widely used chemotherapeutics but are known to be effective in a subset of patients. Identification of this responder patient cohort using the biomarkers will allow implementation of effective personalized medicine for the use of taxanes for breast cancer, breast cancer brain metastasis and GBM.

Taxanes are around 5000 fold more potent compared to the standard of care drug Temozolomide (TMZ) used to treat GBM. With the use of effective delivery methods like ultrasound, blood brain permeable taxane conjugate formulations, repurposing of taxanes to the cancers of brain is becoming feasible and hence the use of biomarker is becoming more relevant to help rescue the failed or failing of large number of clinical trials using taxane formulation/s.

Currently, there are no clinically available biomarkers for predicting response to taxanes hence this biomarker panel discovered by combining functional CRISPR screen and taxane treated TCGA patient survival data, holds promise to guide the patient selection for treatment with taxanes.

As noted above, taxanes are very potent and very widely used chemotherapeutics across different cancers like breast, ovary, lung, pancreas, prostate, etc. Although, there is difference in the individual's response across all the cancers treated with taxanes, there are no clinically available biomarkers for predicting response to taxanes. Therefore, availability of the clinically reliable biomarker panel is the necessity to maximize benefits from the taxane chemotherapy. Using biomarker based approach to stratify patients and guide personalized chemotherapy, will have long term benefits to the patients and also help rescue the failed or failing of large number of clinical trials using taxane or taxane formulation/s in breast cancer, breast cancer brain metastasis and GBM patients. Our biomarker panel also has a great potential to be extrapolated to other cancers treated with taxanes and other microtubule inhibitors including but not limited to vinorelbine.

### EXAMPLES

The following examples are illustrative and should not be interpreted to limit the scope of the claimed subject matter.

### Translocon-Associated Protein Subunit SSR3 Determines and Predicts Response to Paclitaxel in Breast Cancer and Glioblastoma

Paclitaxel (PTX) and taxanes are among the most commonly used chemotherapeutic drugs for cancer. These drugs are potent, exerting cytotoxicity to cancer cells at nanomolar concentrations. Thus, PTX is the basis of chemotherapeutic regimens for breast, pancreatic, lung and ovarian carcinomas (Gradishar et al., 2005; Markman et al., 2003; Rosell et al., 2002; Von Hoff et al., 2013)

Gliomas and specifically glioblastomas (GBMs) are the most common and most deadly of all primary brain tumors in adults. Unfortunately, despite extensive research and the use of multimodal therapeutic strategies, the median overall survival time is 15-20 months from diagnosis (Stupp et al., 2017). PTX is one of the most potent drugs against GBM in-vitro, with an IC50 1400-fold lower temozolomide (Zhang et al., 2020). However, the poor penetration of PTX across the blood-brain barrier (BBB) has limited its use for brain tumors (Chamberlain and Kormanik, 1995). In this context, there are multiple emerging strategies to deliver PTX into the brain, including peptide-drug conjugate, PTX polymer microspheres/nanoparticles, convection-enhanced delivery and focused ultrasound (Drappatz et al., 2013; Gabikian et al., 2014; Koziara et al., 2004; Kumthekar et al., 2020; Lidar et al., 2004; Shen et al., 2017; Walter et al., 1994; Zhang et al., 2020). In this context, we have launched a Phase 1-2 clinical trial for recurrent GBM to evaluate the safety and efficacy of enhancing the delivery of systemic PTX using a skull-implantable ultrasound device that transiently opens the BBB (NCT04528680).

PTX is highly potent, yet there is a spectrum of susceptibility to this drug within individual tumors within a given cancer. This is particularly important for GBM, as these tumors are notorious for their molecular heterogeneity and unpredictable response to therapies. Thus, even if the challenge of PTX delivery across the BBB is solved, the efficacious implementation of PTX-based therapy for GBM will be significantly influenced by the identification of tumors that respond to this drug. Despite the widespread use of PTX for breast cancer, among other malignancies, and our approach to repurpose it for GBM, there are no predictive biomarkers to inform which patients will benefit from this therapy. Understanding the biological basis of individual response to this drug, and developing predictive biomarkers will allow a personalized implementation of PTX-based therapy for GBM, and refine the indication for using this drug in common scenarios such as breast cancer in which PTX is readily used. In this context, we present an unbiased approach that incorporates causality using CRISPR screen, with correlative evidence using patient datasets, to discover predictive biomarkers for response to PTX.

### Results

### PTX is a Potent Drug Against Several Cancers but Exhibits a Variable Susceptibility Across Individual Tumor Cell Lines.

PTX was found to be one of the most potent drugs among the commonly used chemotherapeutics across multiple cancer cell lines (FIG. 9A) from the Genomics of Drug Sensitivity in Cancer Project (GDSC) database (Yang et al., 2013). Further, we found a comparable susceptibility range for human glioma cell lines as observed for lung and breast cancers in which PTX is clinically used (FIG. 9B).

Given the known molecular heterogeneity of human cancer, and its characteristic variable response to therapies, we investigated the susceptibility of individual breast cancer and glioma cell lines to PTX using GDSC (Yang et al., 2013). This analysis revealed that cell lines from these tumors exhibit a wide range of susceptibility to PTX (FIGS. 9C and D). This suggests that the molecular characteristics of individual tumors may be more important to predict susceptibility to PTX than the overall cancer entity.

Genome-Wide CRISPR Knock-Out Screen Reveals Genes that Confer Susceptibility to Paclitaxel.

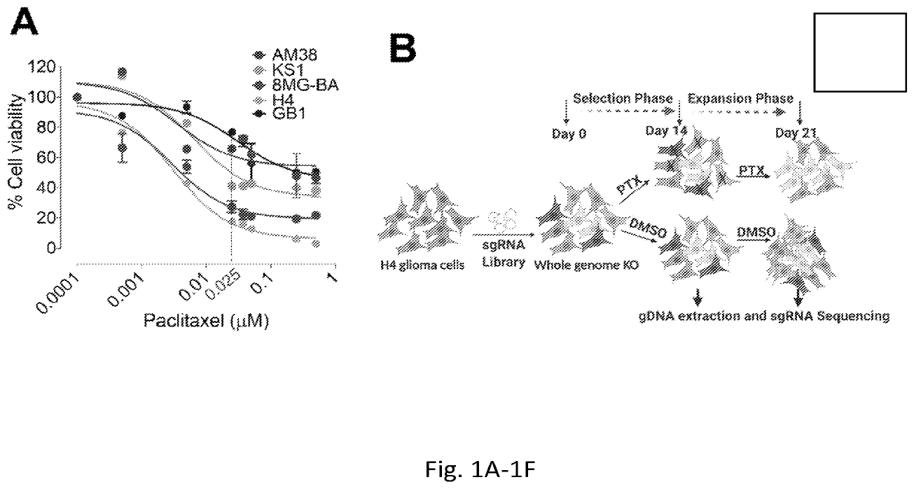

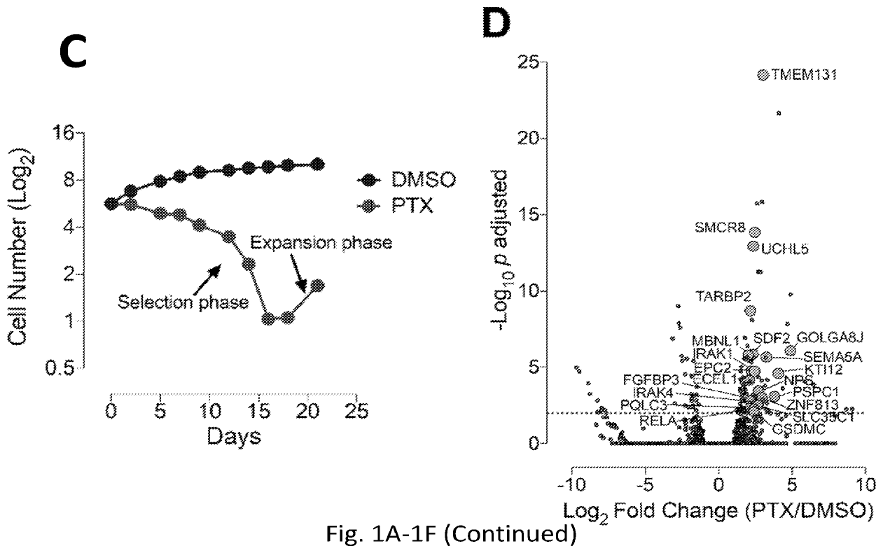

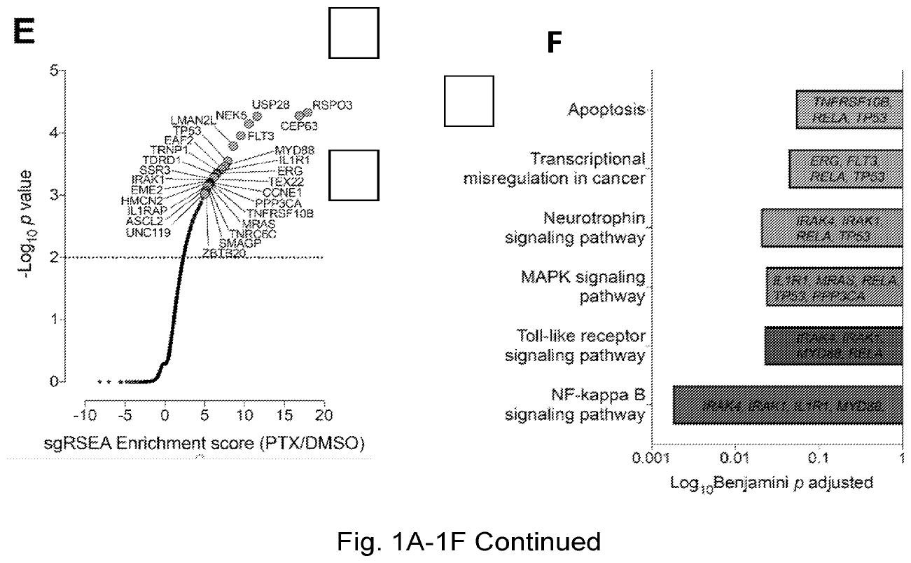

To identify genes that influence PTX susceptibility in glioma, we first characterized different glioma cell lines for response to PTX (FIG. 1A). This analysis revealed the human glioma cell line H4 as the most sensitive amongst those tested. Therefore, H4 cells were used to perform PTX-genome-wide CRISPR knock-out screen. H4 cells transduced with a genome-scale gRNA library were subjected to treatment with 0.025 μM PTX or DMSO. The PTX concentration used was the lowest that was sufficient to kill most of the sensitive cell lines, but had minimal effect on the resistant cell lines (FIG. 1A). Cells were continuously treated with PTX or DMSO for 21 days. Over the course of the screen, we observed a selection phase in which PTX-treated cells declined in number, followed by an expansion phase in which resistant clones increased in number (FIGS. 1B and C). Along with Day 0 (post-puromycin selection), 20% of the cells were collected on day 14 (D14) from both PTX and DMSO groups as an intermediate time point. The terminal time point was day 21 (D21), at which period there was an expansion of resistant clones in the PTX-treated group. The gRNAs enriched in PTX group were determined by comparing gRNA from PTX-treated samples (D14 and D21) against DMSO-treated samples (D14 and D21) using DESeq and sgRSEA R algorithms (FIGS. 1D and E). Using an adjusted p<0.01 for DESeq and p<0.001 for sgRSEA R, we obtained 51 genes enriched in PTX compared to the DMSO group (Table 1 and Table 2). Gene ontology analysis of these 51 genes revealed that PTX treatment led to the selection of KO clones for genes involved in pathways like NF-kappa B signaling (Benjamini-Hochberg (BH) adjusted p=0.0017), toll-like receptor signaling (BH p=0.022), MAPK signaling (BH p=0.023), neurotrophin signaling (BH p=0.020), transcriptional misregulation in cancer (BH p=0.043) and apoptosis (BH p=0.053) (FIG. 1F). Along with previously reported genes involved in PTX susceptibility like P53 (Lanni et al., 1997), apoptosis markers like FADD, caspase 8, and BAX (Janssen et al., 2007; Mielgo et al., 2009; Shimada et al., 2004), the screen also identified several novel genes that had not been previously implicated in susceptibility to PTX.

TABLE 1
DESeq2 R analysis PTX vs. DMSO

| Deseq enriched | Log₂(FoldChange) | p adjusted value |
|---|---|---|
| GOLGA8J | 4.888321537 | 8.26595E−07 |
| KTI12 | 4.070513943 | 2.4898E−05 |
| PSPC1 | 3.81140611 | 0.000800033 |
| SEMA5A | 3.250357956 | 2.08901E−06 |
| MUC13 | 3.196404087 | 2.3287E−06 |
| TMEM131 | 3.032569216 | 7.27191E−25 |
| ZNF813 | 2.94846454 | 0.000961101 |
| NPS | 2.71994961 | 0.000334247 |
| SLC35C1 | 2.678956738 | 0.002942519 |
| IRAK4 | 2.535152893 | 0.002671435 |
| SMCR8 | 2.468837897 | 1.4238E−14 |
| EPC2 | 2.446119299 | 1.8011E−05 |
| GSDMC | 2.401613863 | 0.008096265 |
| GRIN3B | 2.392301396 | 2.46195E−06 |
| NPPC | 2.386781416 | 0.006472314 |
| RELA | 2.379156337 | 0.005479773 |
| UCHL5 | 2.363379819 | 1.12429E−13 |
| SDF2 | 2.335658158 | 1.34275E−06 |
| IRAK1 | 2.231638065 | 1.74339E−05 |
| TARBP2 | 2.159638963 | 1.98722E−09 |
| PQLC3 | 2.152132814 | 0.004835945 |
| FGFBP3 | 2.141883974 | 0.001561506 |
| TEX22 | 2.125939511 | 8.52633E−05 |
| ECEL1 | 2.09180191 | 6.81106E−05 |
| MBNL1 | 2.017270026 | 1.57925E−06 |

TABLE 2
sgRSEA R analysis PTX vs. DMSO

| sgRSEA R enriched | Enrichment Score | p value |
|---|---|---|
| RSPO3 | 17.92435624 | 5E−05 |
| CEP63 | 16.89861281 | 5E−05 |
| USP28 | 11.57833323 | 5E−05 |
| NEK5 | 10.56836826 | 7E−05 |
| FLT3 | 9.516230539 | 0.0001 |
| LMAN2L | 8.589580249 | 0.0002 |
| TP53 | 7.89116356 | 0.0003 |
| MYD88 | 7.540699573 | 0.0003 |
| EAF2 | 7.135193262 | 0.0004 |
| IL1R1 | 6.528154085 | 0.0004 |
| TRNP1 | 6.46920803 | 0.0005 |
| ERG | 6.407892859 | 0.0005 |
| TDRD1 | 6.228808574 | 0.0005 |
| TEX22 | 6.16307786 | 0.0005 |
| SSR3 | 5.6727358 | 0.0006 |
| CCNE1 | 5.599775471 | 0.0006 |
| IRAK1 | 5.557704187 | 0.0007 |
| EME2 | 5.513206936 | 0.0007 |
| PPP3CA | 5.467964959 | 0.0007 |
| HMCN2 | 5.415355738 | 0.0007 |
| TNFRSF10B | 5.406043059 | 0.0007 |
| MRAS | 5.370384987 | 0.0007 |
| IL1RAP | 5.188289268 | 0.0008 |
| TNRC6C | 5.179472295 | 0.0008 |
| ASCL2 | 5.1234743 | 0.0009 |
| SMAGP | 5.11732294 | 0.0009 |
| UNC119 | 5.04573486 | 0.0009 |
| ZBTB20 | 5.00104397 | 0.001 |

Identification of Putative Predictive Biomarkers for Paclitaxel Response by Combining CRISPR Screen Results with Outcomes from Taxane-Treated Breast Cancer Patients.

The functional implication by the CRISPR KO screen does not necessarily mean that the baseline expression of a gene hit on this experiment is a good predictive biomarker for response to a drug, as genes that are involved in susceptibility might be induced by drug treatment. Therefore, to identify a subset of genes functionally implicated whose baseline expression predicts response to PTX, we refined our candidate gene list through Cox Proportional-Hazards Model (Cox) analysis using the TCGA breast cancer dataset (Goldman et al., 2020), including patients that had annotation of chemotherapy used, RNA-seq-based expression data for tumor tissue, and overall survival. We chose breast cancer since PTX is commonly used in this setting, allowing robust correlation with patient outcomes for biomarker discovery. On the other hand, discovery of a predictive biomarker for breast cancer would allow immediate validation and application of this discovery for patients that routinely get this drug. We divided the patients into 1. Patients treated with taxanes 2. Patients who did not receive any chemotherapy and 3. Patients treated with drugs other than taxanes. We queried the expression of 51 genes identified as CRISPR screen hits (hit calling criteria as summarized in FIG. 2A). Correlation analysis of expression of these genes with chemotherapy used (taxanes, non-taxane drugs or no chemotherapy) with overall survival was analyzed in a continuous fashion by Cox (Table 3 and Table 4). The Cox analysis was used to obtain the list of significantly predictive genes and the bootstrap analysis was used to compute the p-value (0.0003 & 0.0023) relative to a random list of 51 genes.

TABLE 3
Taxanes treated patients compared to untreated patients

| | Gene | | Treatment | | Interaction | |
|---|---|---|---|---|---|---|
| | Coefficient | | Coefficient | | Coefficient | |
| | HR of | ‘p’ | HR of | ‘p’ | HR of | ‘p’ |
| Genes | gene | gene | paclitaxel | paclitaxel | interaction | interaction |
| CEP63 | 1.5528625 | 0.0669 | 1.3904E+21 | 0.0014 | 0.042485 | 0.0012 |
| SSR3 | 1.1806912 | 0.3918 | 3.9018E+22 | 0.0013 | 0.067786 | 0.0011 |
| IRAK4 | 1.5441908 | 0.0355 | 3.8478E+14 | 0.0088 | 0.118079 | 0.007 |
| TMEM131 | 2.2875926 | 0.0001 | 1.3108E+14 | 0.0057 | 0.137243 | 0.0043 |
| EPC2 | 1.5444996 | 0.0178 | 1.3586E+10 | 0.0411 | 0.233447 | 0.0322 |
| MBNL1 | 1.3392369 | 0.0209 | 2.6782E+10 | 0.015 | 0.24549 | 0.0111 |
| ZNF813 | 1.0849132 | 0.4576 | 30912.0081 | 0.0404 | 0.454935 | 0.0221 |
| ZBTB20 | 1.1908889 | 0.0167 | 549.2205 | 0.0428 | 0.516851 | 0.0171 |
| TDRD1 | 1.046551 | 0.1144 | 5.32759544 | 0.2015 | 0.731835 | 0.0274 |

TABLE 4
PTX treated patients compared to patients treated with non-taxane drugs

| | Gene | | Treatment | | Interaction | |
|---|---|---|---|---|---|---|
| | Coefficient | | Coefficient | | Coefficient | |
| | HR of | ‘p’ | HR of | ‘p’ | HR of | ‘p’ |
| Genes | gene | gene | paclitaxel | paclitaxel | interaction | interaction |
| CEP63 | 0.780906 | 0.505 | 1.22379E+17 | 0.0137 | 0.082761 | 0.0144 |
| TDRD1 | 1.034481 | 0.4185 | 17.44407414 | 0.031 | 0.747964 | 0.0394 |
| SSR3 | 1.283384 | 0.3752 | 3.69191E+23 | 0.001 | 0.064998 | 0.0012 |
| IRAK4 | 1.234665 | 0.5143 | 3.00745E+13 | 0.0202 | 0.149718 | 0.0212 |
| MBNL1 | 1.179511 | 0.4442 | 7242990424 | 0.0276 | 0.284677 | 0.0292 |

9/51 genes were found to have a significant interaction coefficient between the drug used and gene expression on overall survival, as were only correlated with survival in taxane-treated patients but not in the untreated group (FIG. 2B). 5/51 genes were found to have significant interaction coefficient between the drug used and gene expression on overall survival when considering the taxane-treated group and the non-taxane chemotherapy group (FIG. 2C). Further, the bootstrap analysis showed 9/51 (18%) genes from the CRISPR screen list to be correlated with survival in taxane-treated and not in untreated patient cohorts (p=0.0003). Similarly, 5/51 (10%) genes from the CRISPR screen list were found to correlate with survival in taxane-treated but not in non-taxane chemotherapy treated patients (p=0.0023) (FIG. 2D). Thus, our approach of combining CRISPR screen with clinical data from TCGA breast cancer dataset led to a list of putative biomarkers that predicted as well as contributed to susceptibility to PTX.

### Baseline Expression of SSR3 Predicts Response to PTX in Breast Cancer and GBM

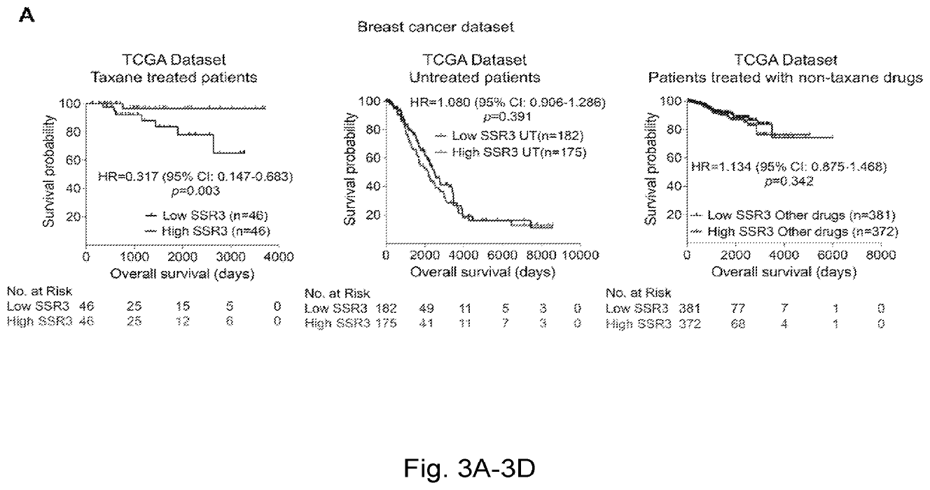

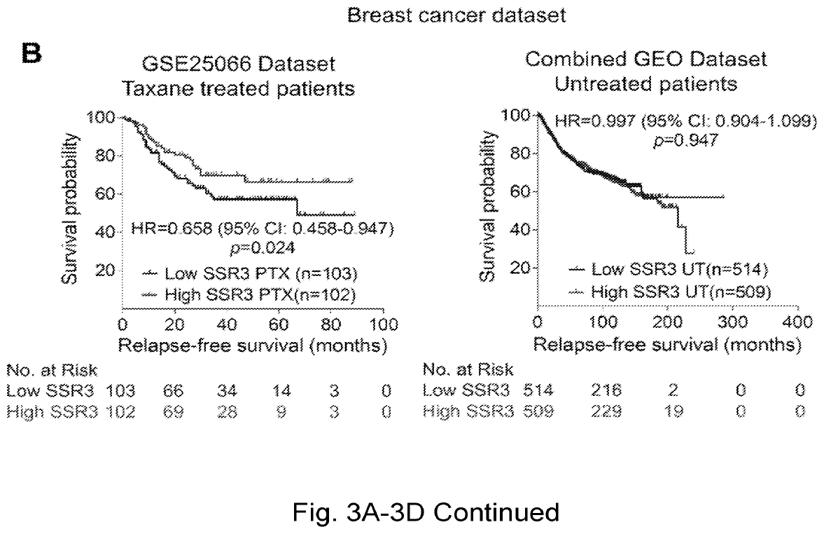

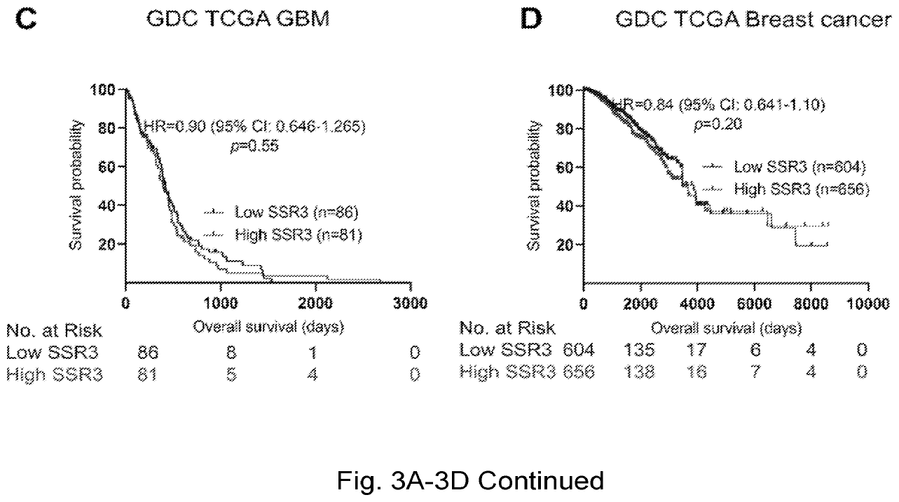

Baseline expression of SSR3 (also known as translocon-associated protein gamma-TRAPγ) was found to be predictive for response to taxanes in two independent breast cancer datasets, among the final list of putative biomarkers investigated from the CRISPR screen (FIGS. 3A and B). For correlation between SSR3 with survival or relapse-free survival, we used a Cox proportional hazards ratio analysis. Indeed, whereas 9/51 putative biomarkers were identified using the TCGA breast cancer dataset as the discovery dataset, only 1/9 (SSR3), was found to be predictive in an independent validation cohort. In the validation cohort (GSE25066 dataset) which includes only taxane-treated patients (Hatzis et al., 2011; Itoh et al., 2014), high SSR3 expression showed significant association with relapse-free survival. In contrast, no association between relapse-free survival and SSR3 expression was observed in the combined GEO datasets (GSE16716, GSE19615, GSE31519, GSE37946, GSE45255 and GSE65194) which is composed of patients who have not received hormonal therapy or chemotherapy (Maire et al., 2013a; Maire et al., 2013b; Maubant et al., 2015). For the validation cohort, among the 9 putative biomarker genes, 6 were not significantly predictive of relapse-free survival (CEP63, IRAK4, TMEM131, MBNL1, ZBTB20, and TDRD1) and 2 of these genes (EPC2 and ZNF813) did not have gene expression data available for the analysis. Thus, SSR3 was predictive of response to taxanes in two independent breast cancer datasets. Although, SSR3 was predictive of response to taxanes, SSR3 RNA expression alone (independent of the chemotherapy received) was not prognostic of a favorable outcome in either GBM or breast cancers (FIGS. 3C and D).

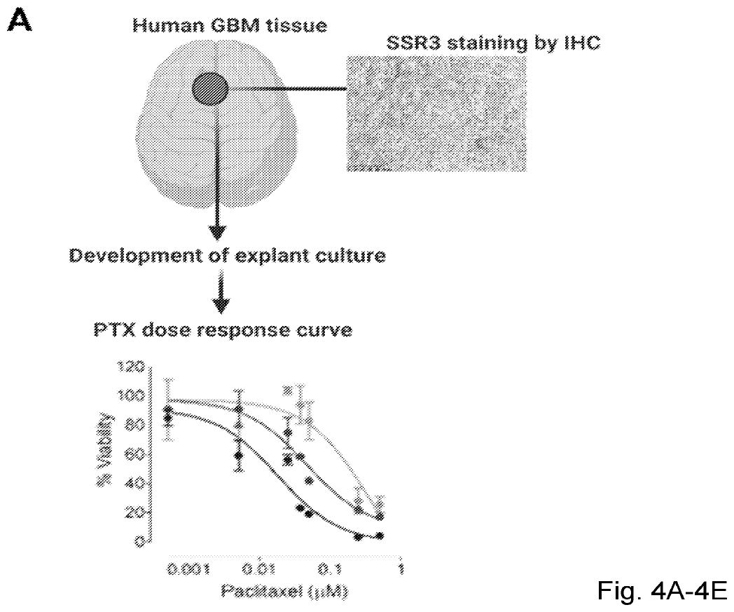

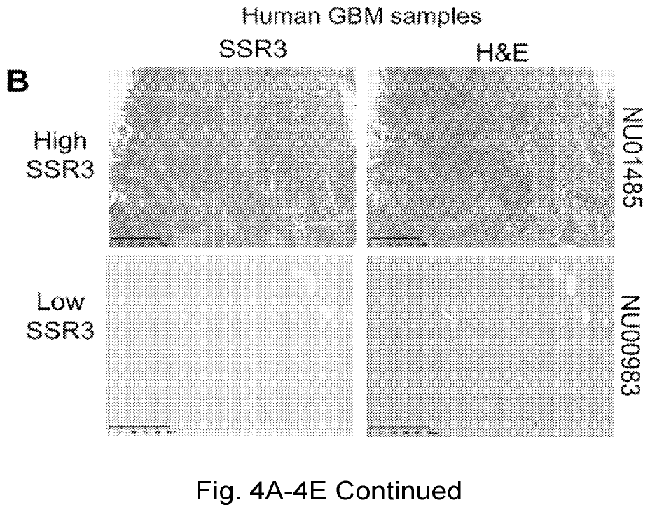

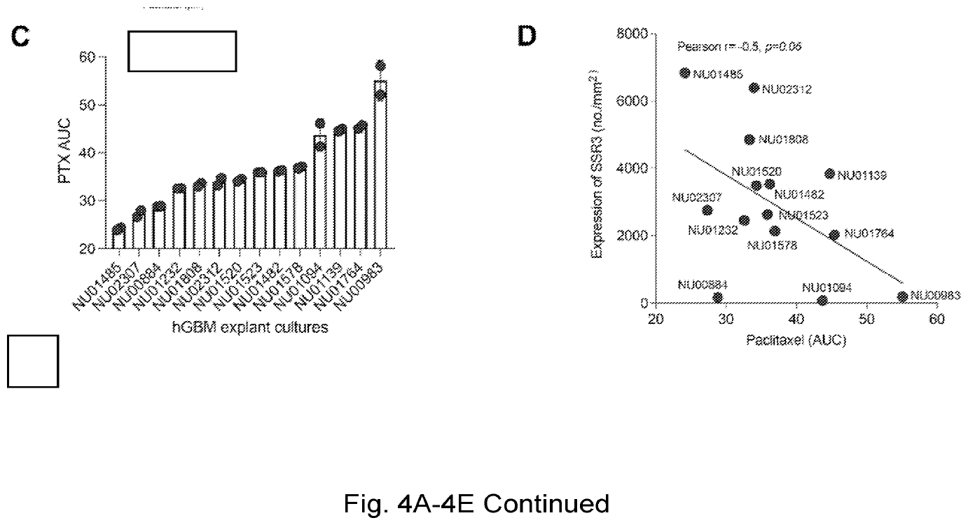

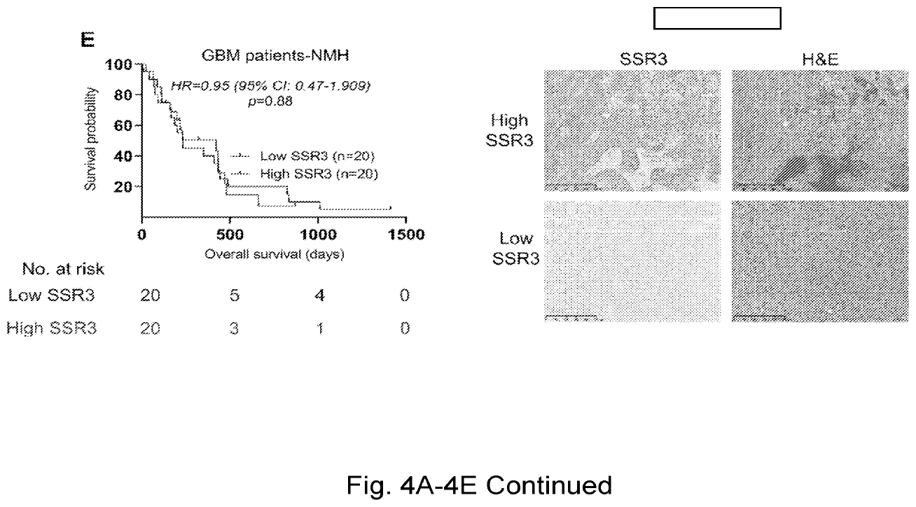

Next we determined if the expression of SSR3 correlates with susceptibility to PTX in human gliomas ex-vivo. To do this, we obtained freshly resected GBM tumors and divided into two parts (FIG. 4A)—one part was used to stain for SSR3 using immunohistochemistry (FIG. 4B, SSR3 immunohistochemistry titration is shown on FIG. 10) and the other part was used to develop primary explant culture. These explant cultures were then characterized for susceptibility to PTX by PTX dose-response curves within 3-5 passages after their establishment. The explant cultures derived from GBM patients demonstrated a range of susceptibility to PTX (FIG. 4C) and a trend for a negative correlation (Pearson R=−0.5, p=0.06, n=14) was found between SSR3 protein expression (determined using quantitative IHC measurement of the tumor region (no./mm²) and susceptibility to PTX (AUC values) (FIG. 4D). Further, when we stained 40 recurrent GBM tumors for SSR3 protein expression and correlated its expression with survival, we did not find any significant association between SSR3 protein expression and overall survival (FIG. 4E). This is consistent with the lack of association between SSR3 transcript expression and survival on our TCGA-based analysis (FIG. 3C), indicating that SSR3 is not a prognostic marker in GBM. The staining for SSR3 on these tumors reinforced that GBMs exhibit a spectrum of SSR3 expression that can be detected by quantitative analysis of immunohistochemistry. This method can be used to identify patients that may respond to PTX treatment.

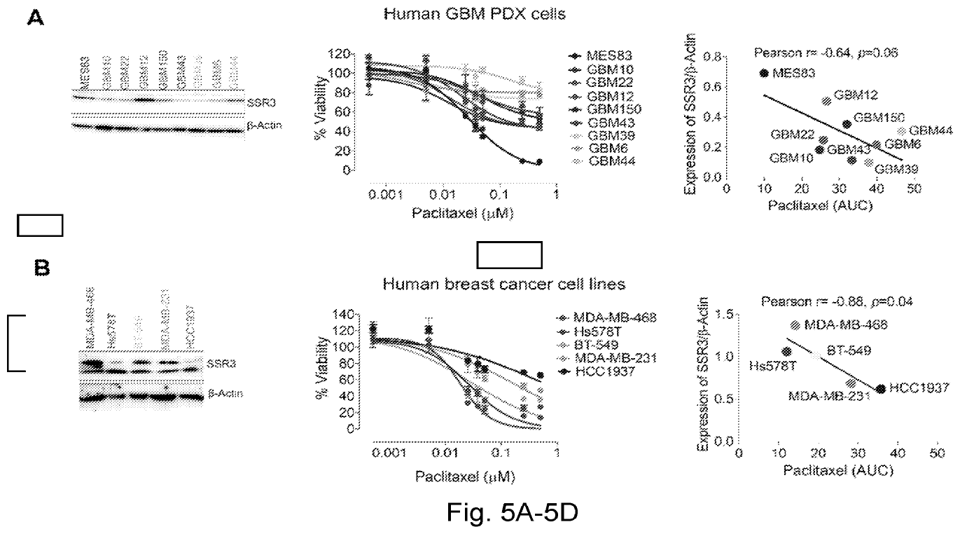

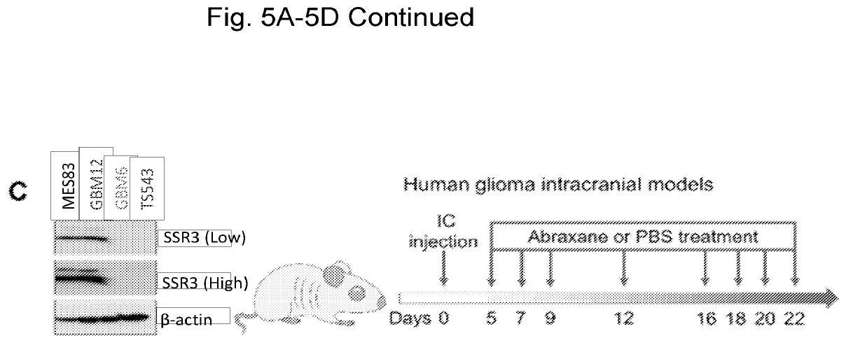

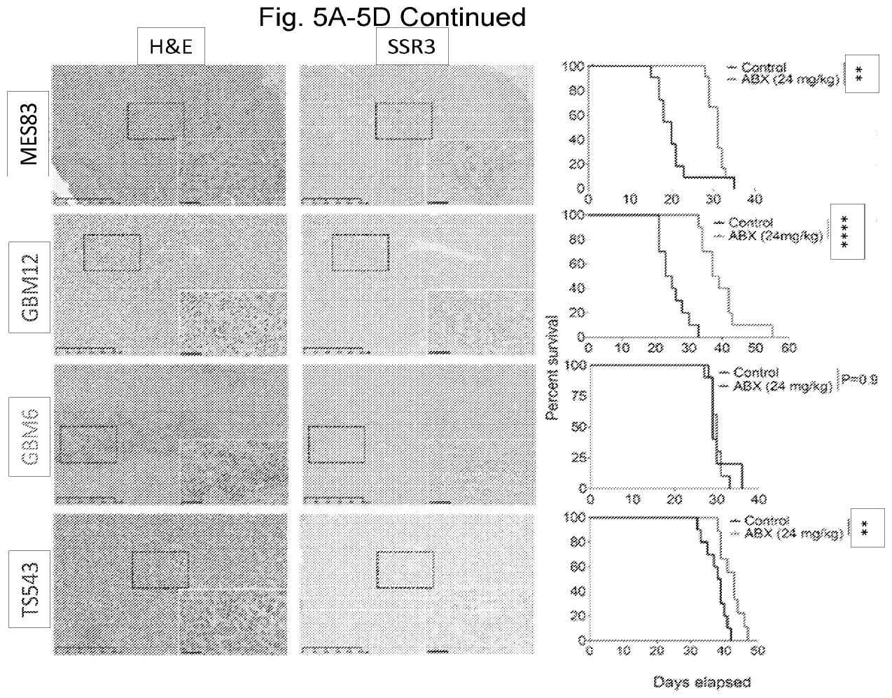

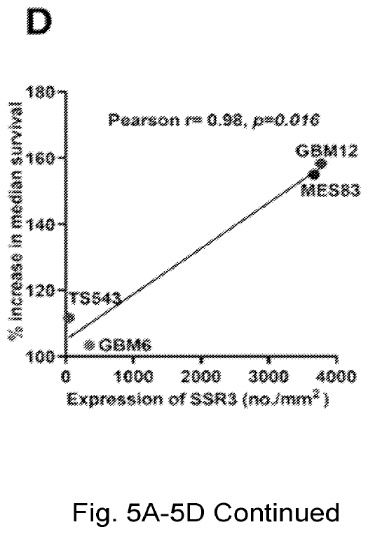

Further, we characterized breast cancer cell lines and glioma patient-derived xenografts (PDX) cells for PTX susceptibility. Area under the curve (AUC) value was determined for each of these lines as a measure of response to PTX treatment. Correspondingly, SSR3 protein levels were determined for these cell lines by western blot. A negative correlation was observed between SSR3 protein levels (image J quantification) and resistance to PTX (AUC values) trending towards significance in the case of glioma PDX cell lines (r=−0.64, p=0.06, n=9) (FIG. 5A). Similarly, analysis of breast cancer cell lines revealed a negative correlation between SSR3 protein levels and resistance to PTX (r=−0.88, p=0.04, n=5) (FIG. 5B). Next, we investigated whether baseline expression of SSR3 is predictive of response to PTX in-vivo across intracranial glioma models. Four human intracranial glioma xenograft models with different SSR3 protein expression determined by western blot and immunohistochemistry, were treated with albumin-bound PTX. Survival curves for MES83 and GBM12 were adopted from (Zhang et al., 2020). MES83 (median survival extended from 20 days to 31 days, p=0.0041) and GBM12 (median survival extended from 24 days to 38 days p<0.0001), which express higher levels of SSR3 demonstrated better response to PTX. On the other hand GBM6 (median survival extended from 29 days to 30 days, p=0.77) and TS543 (median survival extended from 38.5 days to 43 days, p=0.0050) which expresses relatively lower levels of SSR3 demonstrated poor response to PTX (FIG. 5C). The percentage of increase in median survival following treatment with PTX correlated with SSR3 expression (no./mm²) (r=0.98, p=0.016, FIG. 5D). These observations further validate the predictive potential of baseline SSR3 expression for response to PTX treatment in glioma and breast cancer models.

### SSR3 Expression Determines Susceptibility to Paclitaxel in Breast Cancer and Glioma Cells.

To investigate the contribution of each of the putative biomarkers that resulted from the overlap between CRISPR screen analysis and the TCGA breast cancer clinical dataset, we proceeded to validate these genes experimentally through knock-out and assessment of response to PTX. For this, we focused on 8 of the 9 genes since TDRD1 had negligible transcript levels across all GBM and breast cancer samples from TCGA (FIGS. 11A and B), which makes it a poor biomarker candidate. We knocked out (KO) each of the remaining genes individually in PTX sensitive glioma (H4) and breast cancer (MDA-MB-468) cell lines using the CRISPR-cas9 system. The KO of 8/8 genes was confirmed using quantitative real-time PCR by comparing the expression of the gene of interest to that of their respective non-targeting control clones. To test whether KO of these genes renders cells resistant to PTX treatment, we performed a PTX dose response curve. KO of the 7 genes (SSR3, IRAK4, TMEM131, EPC2, MBNL1, ZNF813 and ZBTB20) independently conferred resistance to PTX as determined by the area under the curve (AUC) for the dose response curves as compared to the control clones for both glioma and breast cancer cell lines (FIGS. 12A-B and 13A-B). However, knockout of CEP63 (1/8) gene conferred resistance in glioma cells and sensitivity in breast cancer cells (FIGS. 12A-B and 13A-B).

The strong correlation between SSR3 with response to taxanes and the single-gene KO data confirmed that SSR3 KO had the strongest effect on susceptibility among all the genes tested.

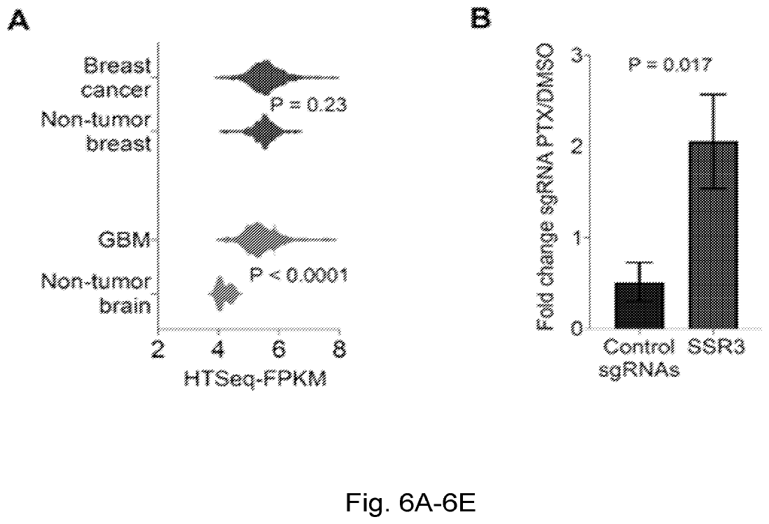

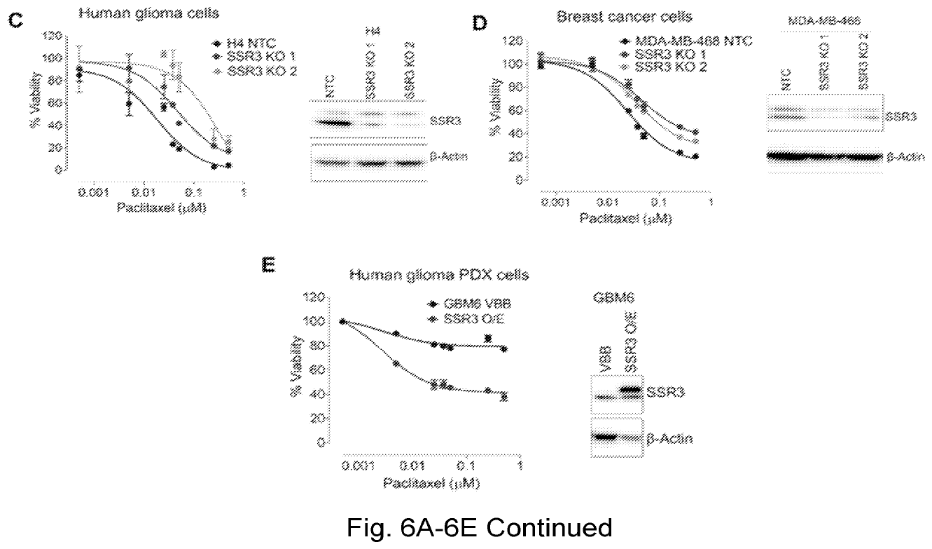

SSR3 is overexpressed in GBM as compared to the normal brain tissue, whereas in breast cancer SSR3 shows a spectrum of expression as compared to the non-tumor breast tissue (FIG. 6A). In the PTX CRISPR screen, gRNAs for SSR3 were 2 fold more enriched in PTX group as compared to its DMSO control group (FIG. 6B). KO of SSR3 in PTX sensitive glioma cell line H4 and breast cancer cell line MDA-MB-468, rendered these cells resistant to PTX (FIGS. 6C and D). For MDA-MB-468 cells, SSR3 KO 2 showed higher susceptibility and for H4, SSR3 KO 1 showed higher susceptibility, hence these clones were used for subsequent experiments. Correspondingly, overexpression of SSR3 in PTX resistant-SSR3 low-GBM6 cells led to significant increase in susceptibility of these cells to PTX (FIG. 6E). In line with the increased in-vitro susceptibility to PTX, intracranial tumors developed with GBM6 overexpressing SSR3, improved their response to albumin-bound PTX Abraxane (ABX) (median survival extended from 29 days to 38 days, p<0.0016) as compared to the GBM6 vector control cells (median survival extended from 47 days to 50 days, p<0.01) (FIGS. 7A-C). Similarly, mammary fat pad tumors developed from MDA-MB-468 SSR3 KO cells showed poorer response to ABX as compared to its vector control cells (p<0.018) (FIGS. 7D-F). These data confirms the causal role of SSR3 in regulating susceptibility to PTX in both breast cancer and glioma cells.

### Phosphorylation of IRE1α Contributes to SSR3-Mediated Susceptibility to PTX in Glioma Cells.

Basal levels of SSR3 were found to determine response to PTX. In line with this, SSR3 was not induced by 48 h of treatment with PTX in PTX sensitive cell lines H4 NTC and MES83 and in PTX resistant GBM6 cells (FIG. 14A). SSR3 is a subunit of translocon-associated protein (TRAP) complex, localized in the endoplasmic reticulum (ER) lumen and is primarily involved in the folding and transport of proteins destined to ER (Fons et al., 2003; Gorlich et al., 1992; Hartmann et al., 1993). Inhibition of TRAP complex function by depletion of SSR3 was found to influence ER stress related unfolded protein response (UPR pathway) (Nagasawa et al., 2007). ER stress response and ER transport machineries are shown to interact with each other via UPR gene IRE1α (Acosta-Alvear et al., 2018). Moreover, SSR3 KO mice and IRE1α KO mice have shown to confer similar phenotypes on the development of placenta (Iwawaki et al., 2009; Yamaguchi et al., 2011). With this in mind, we investigated IRE1α and other ER stress markers in the context of susceptibility to PTX. We observed a correlation between SSR3 and IRE1α levels (Pearson r=0.88, p=0.001), n=9) but not with other ER stress markers like BIP and PERK in glioma PDX cell lines (FIG. 14B-E). A correlation was also observed between SSR3 protein levels and p-IRE1α in glioma PDX cell lines (Pearson r=0.75, p=0.019, n=9, FIG. 8A). In H4 glioma cells, SSR3 KO led to the loss of phosphorylation of IRE1a, regardless of PTX treatment. Further, knockout of IRE1α in PTX sensitive H4 glioma cells rendered them resistant to PTX (FIG. 8B). We performed a rescue experiment in GBM6 SSR3-overexpressing cells that have acquired susceptibility to PTX, to investigate whether IRE1α loss would restore the PTX resistance phenotype found in GBM6 wild-type cells. We found that knockout of IRE1α in SSR3-overexpression background rendered the cells resistant to PTX (FIG. 8C). On the other hand, the knockout of IRE1α in PTX-resistant-wild-type GBM6 cells (SSR3 low), did not change its susceptibility to PTX (FIG. 8D). Based on our observation, we hypothesize that SSR3 stabilizes the phosphorylation of IRE1a, which ultimately contributes to the susceptibility of glioma cells to PTX (FIG. 8E).

Next, we investigated the effect of SSR3 KO on microtubule polymerization following PTX treatment, as this is the canonical mechanism of action of PTX. As previously reported, PTX led to stabilization of microtubule polymerization, yet no significant difference was seen on the microtubule polymerization in the presence of PTX between control and SSR3 KO cells (FIG. 15). These results suggest that SSR3's modulation of PTX susceptibility is associated with the phosphorylation of IRE1α and is either downstream or independent of microtubule polymerization.

Along with the modulation of IRE1α phosphorylation, SSR3 also contributed to the proliferation potential of the cells. Overexpression of SSR3 in GBM6 conferred increased proliferation potential while the reduction in proliferation potential was observed upon knockout of SSR3 in H4 and MDA-MB-468 cells (FIG. 16A-C). This was also reflected in in-vivo survival studies, where animals injected with GBM6 overexpressing SSR3 cells (median survival=29 days) reached the endpoint earlier as compared to their vector control group (median 47 days, p<0.0001) in the absence of any treatment (FIG. 16D). In line with this, a correlation was observed between the % Ki67 labeling index (as determined by the pathologist) and SSR3 expression on GBM specimens (r=0.6, p=0.0036, n=20) (FIG. 16E). However, no correlation was seen between % Ki67 positivity and susceptibility to PTX (determined by dose-response assay on explant cultures developed from fresh GBM tissues) (FIG. 16F). Similarly, no correlation was seen between doubling time and susceptibility to PTX in both glioma PDXs and breast cancer cell lines (FIGS. 16G and H). These observations suggest that an increase in proliferation potential conferred by SSR3 may not contribute towards PTX susceptibility.

### Discussion

Given the limited efficacy of the current therapies for GBM, novel therapies are desperately needed. In particular, in the case of recurrent GBM, there are no established treatments that prolong survival. Unfortunately, virtually all GBM patients recur, thus effective therapies for this disease stage are warranted. The exquisitely low concentration of PTX necessary to achieve tumor cell death, makes this agent among the most potent drugs for GBM. In this context, several preclinical studies and clinical trials have evaluated the use of PTX for gliomas (Chamberlain and Kormanik, 1995; Chang et al., 1998; Drappatz et al., 2013; Fellner et al., 2002; Fetell et al., 1997; Heimans et al., 1995; Lidar et al., 2004; Postma et al., 2000; Prados et al., 1996; Zhang et al., 2020). However, pharmacologic data and clinical investigations have shown that PTX does not cross the BBB (Fellner et al., 2002; Heimans et al., 1995; Lesser et al., 1995; Zhang et al., 2020). Yet, given the preclinical therapeutic promise of PTX for gliomas, emerging technologies and approaches to overcome the blood-brain barrier have led to renewed interest in PTX for glioma therapy. Numerous approaches are being pursued including the use of convection-enhanced delivery, biodegradable wafers that release PTX, peptide-drug conjugates, nanoparticle-drug coupling to enhance the PTX penetration across the BBB, and most recently the use of ultrasound for transient BBB opening (Drappatz et al., 2013; Lidar et al., 2004; Zhang et al., 2020). Indeed, enhancing delivery of PTX to the peritumoral brain might be key for efficacious use of this drug for gliomas as this is the compartment where infiltrating glioma cells reside after the bulk of the tumor is resected, where approximately 80-90% of GBM recurrences originate (Oh et al., 2011; Rapp et al., 2017; Wick et al., 2008).

We recently showed that the clinically available nanoparticle albumin-bound paclitaxel formulation (nab-paclitaxel, AbraxaneR, ABX) can be safely delivered across the BBB using transient BBB opening through brain ultrasound treatment with concomitant administration of microbubbles (Zhang et al., 2020). The implementation of ultrasound-based BBB opening in humans requires that the sound waves penetrate or bypass the thick human skull, a challenge that is been overcome by the use of an ultrasound device that is implanted on a window in the skull during surgery for resection of GBM (Carpentier et al., 2016; Idbaih et al., 2019; Sonabend and Stupp, 2019). In this context, we are conducting a first-in-human Phase 1-2 clinical trial to evaluate the safety, PTX distribution and early signs of efficacy of Abraxane® delivery using skull implantable ultrasound for recurrent GBM (NCT04528680). In mice, we showed that following systemic delivery of ABX, ultrasound therapy can enhance the brain penetration of PTX by 3-5 fold, achieving parenchymal drug concentrations of approximately 0.3-0.4 μM (Zhang et al., 2020). The concentrations achieved by these means should be interpreted carefully as there are limitations to comparing the biodistribution and dosing between mice and humans, and there might be differences between GBM response to PTX in patients vs that of PTX susceptibility in vitro. Yet, these concentrations serve as an initial parameter to designate glioma cells with a PTX IC50 greater than 0.3-0.4 μM as resistant and those with IC50 lower than this as sensitive to this drug.

Taxanes are among the most commonly used chemotherapeutics used for cancer as these drugs are indicated for ovarian, breast, lung cancer, as well as pancreatic cancer and Kaposi's sarcoma, and are also used off-label for many other scenarios in oncology (Lerose et al., 2012). As a result of the use of these drugs for very prevalent tumors such as lung and breast carcinomas, there are many patients managed with these agents.

Whereas taxanes are highly potent, there is a known variability in response to these drugs across tumors (Chamberlain and Kormanik, 1995; Fetell et al., 1997). Indeed, it is estimated that approximately 50% of patients do not experience a/clinical benefit PTX-based treatment (Weaver, 2014). Our study supports these previous observations as approximately half of glioma and breast cancer cell lines we analyzed were relatively resistant to this drug. Thus, whereas the concept of personalized cancer therapy is usually introduced in the context of targeted agents, its implementation for conventional cytotoxic chemotherapeutic agents such as taxanes is of great significance. Based on this, we estimate that roughly half of the patients enrolled in our clinical trial and other cohorts, will have tumors that are resistant to PTX. For this reason, the efficacy-based outcome of our Phase 1-2 study and other clinical trials evaluating means of enhancing delivery of PTX across the BBB for treatment of malignant gliomas (Drappatz et al., 2013; Lidar et al., 2004), is heavily influenced by the composition of susceptible vs resistant tumors, a variable that we cannot control or measure at this time.

Given the need for personalizing the use of PTX, a predictive biomarker for this drug is being investigated by various groups. As the canonical mechanism of action for PTX is stabilization of microtubule polymerization, some studies have explored genes that relate to microtubule assembly and related cytoskeleton biology. Rouzier et al. and colleagues identified expression of microtubule-associated protein tau in conferring resistance to PTX treatment in breast cancer using U133A chips. They showed that knockdown of tau protein rendered the cells susceptible to PTX (Rouzier et al., 2005), However, in the NSABP B28 trial, tau expression correlated with improved outcome independent of whether patients received taxane-containing chemotherapy regimen or not (Pusztai et al., 2009). Another study by Swanton et al. and colleagues using a kinome and ceramidome siRNA screen, identified the role of ceramide transport protein, COL4A3BP in conferring resistance to PTX in ovarian cancer setting (Swanton et al., 2007). The PTX resistant gene signature identified from this study was successfully validated in patients with triple-negative breast cancer (Juul et al., 2010). Whole genome siRNA screen was performed by Whitehurst and colleagues to identify chemosensitizer for PTX i.e. to identify gene targets that specifically reduce cell viability in the presence of sublethal concentrations of PTX. They demonstrated that inhibition of proteasomal subunits chemosensitizes lung cancer cells to PTX (Whitehurst et al., 2007). In an independent study MCL1 expression was shown to confer resistance to anti-tubulin chemotherapeutics like PTX and vincristine (Wertz et al., 2011). Another group has reported the role of solute carrier transporters in conferring resistance to PTX by mediating efflux of the drug (Njiaju et al., 2012). Cyclin G1 levels were determined to be resistant to PTX and ovarian cancer patients with cyclin G1 amplification showing poor post-surgery survival in a taxane treated group (Russell et al., 2012). Gargini et al. showed that overexpression of tau can confer resistance to taxanes since both tau and taxane compete for the same microtubule binding site (Gargini et al., 2020). Several of these efforts identified genes whose expression is associated with resistance to PTX, yet none of these studies has led to a clinically-valid biomarker that predicts susceptibility or response to PTX treatment.

The focus of our study was to identify a predictive biomarker for response to PTX. We used a genome-wide CRISPR KO approach to identify genes that confer susceptibility to PTX. The CRISPR screen led to identification of 51 genes. These 51 genes were then tested for their ability to predict survival of the TCGA breast cancer patients treated with taxanes using Cox analysis. 8/51 genes found to be predictive were validated by single gene KO experiments in glioma and breast cancer cell lines. SSR3 gene was found be most superior biomarker in conferring susceptibility as well as in predicting response to taxane treatment in both glioma and breast cancer models. Our approach of combining CRISPR screen with the publicly available databases led to the identification of a biomarker with causal as well as correlative properties.

Our study suggests that SSR3 expression leads to PTX susceptibility by a mechanism that is either independent or downstream of the effect that PTX has on microtubule polymerization. This conclusion is supported by the fact that the microtubule polymerization pattern following PTX treatment was not influenced by SSR3 KO. Whereas microtubule polymerization has been considered the canonical mechanism of action for PTX cytotoxicity, emerging studies are reporting that PTX can elicit deleterious effects by other mechanisms. For example, studies have described the release of apoptosis-inducing cytokine TNFα and phosphorylation of anti-apoptotic Bcl-2 protein on cancer cells (Husemann et al., 2020; Lanni et al., 1997). Our study shows that SSR3 stabilizes the phosphorylation of IRE1α to predispose the cell to PTX mediated cytotoxicity.

Our approach has several limitations. This includes validation only on publicly available breast cancer databases with limited curation/annotation, where information on previous treatment/s administered, and the small number of samples/subjects tested might not be optimal. More importantly, this analysis is retrospective, and relies on RNA-based expression as opposed to protein levels of SSR3. Further, the glioma explant cultures (FIG. 4C) might not resemble the actual tumor, impacting its direct extrapolation to the response to PTX in patients. Given this, our study serves as a hypothesis-generating report, and the predictive property of SSR3 expression for PTX response will require prospective investigation and validation. Moreover, a single gene might contribute to the susceptibility to a drug, the interaction of multiple putative biomarker candidates may more closely predict and determine susceptibility to taxanes. Although, SSR3 expression appears to be predictive of response to PTX for both GBM and breast cancer, we have only investigated the mechanism by which SSR3 is modulating PTX susceptibility in gliomas. Important molecular differences between these two cancers warrant disease-specific validation of this biomarker and the mechanisms by which SSR3 modulates this response.

Our study to pursue a predictive biomarker for PTX response was motivated by the practical challenge we face in the context of investigating whether this drug might be efficacious for GBM. Identification of susceptible GBM tumors might allow early elucidation of an efficacy signal on our study and other ongoing trials, and eventual refinement on the inclusion criteria to enrich for patients with susceptible tumors in a future trial design. On the other hand, this approach and study is a prelude to prospective validation of SSR3 as a predictive biomarker for PTX in GBM. On a broader perspective, the application of personalized medicine for commonly used chemotherapeutic drugs such as taxanes might have a direct impact on the management of a lot of patients that routinely get these drugs, including a large number of patients that do not benefit from this therapy. On the other hand, personalized use of PTX could also open therapeutic possibilities for patients who have tumors that are susceptible to this drug, that currently do not get this treatment as is not part of the standard chemotherapeutic regimen for their cancer diagnosis.

### Experimental Model and Subject Details

### Cell Lines

Cells were incubated at 36° C. and 5% CO₂. 8MGBA and AM38 cells were grown in Minimum Essential Media (Corning) with 20% Fetal Bovine Serum (GE Health Sciences) and 1% Penicillin/Streptomycin (Corning). H4 cells were grown in in Dulbecco's Minimum Essential Media (DMEM) containing 10% FBS. TS543 cells were grown in NeuroCult NS-A proliferation kit (StemCell Technologies) with 20 ng/ml recombinant human Epidermal Growth Factor (PeproTech), 20 ng/ml recombinant human Platelet Derived Growth Factor-AA (PeproTech), 20 ng/ml recombinant Fibroblast Growth Factor (PeproTech), and 2 μg/ml heparin sulfate (StemCell Technologies). MES83 cells were a generous gift from Ichiro Nakano (University of Alabama). MES83 cells were cultured in Dulbecco's Minimum Essential Media (DMEM) containing 10% FBS and 1% P/S. All other GBM PDX cells were purchased from Mayo Clinic (Scottsdale, AZ, USA) and grown in Dulbecco's Minimum Essential Media (DMEM) containing 10% FBS. Breast cancer cell lines MDA-MB-231, BT549, Hs578T and HCC1937 were kindly gifted by Dr. Maciej Lesniak (Northwestern University). MDA-MB-468 cells were kindly gifted by Dr. Dai Horuichi (Northwestern University). All the cell lines used in this study were profiled for STR and tested for mycoplasma contamination. The list of all the cell lines, primary antibodies and reagents used in this study is described in Table 5.

TABLE 5

| | | |
|---|---|---|
| REAGENT or RESOURCE | SOURCE | IDENTIFIER |

Antibodies

| | | |
|---|---|---|
| Anti-SSR3 antibody produced in rabbit | Sigma-Aldrich | HPA014906 |
| IRE1 alpha [p Ser724] Antibody | Novus biologicals | NB100-2323 |
| IRE1α (14C10) Rabbit monoclonal antibody | Cell signaling technology | 3294S |
| β-Actin (13E5) Rabbit monoclonal antibody | Cell signaling technology | 4970S |
| ER Stress Antibody Sampler Kit | Cell signaling technology | 9956T |
| Monoclonal Anti-α-Tubulin antibody produced in | Sigma-Aldrich | T5168-100UL |
| mouse | | |

Plasmids/Vectors and bacteria

| | | |
|---|---|---|
| CCSB-Broad Lentiviral Expression Collection | Dharmacon-Horizon | ID: OHS6085- |
| | inspired cell solutions | 213574251 |
| lenticrispr v2 plasmid | Addgene-52961 | Sanjana N E et al., |
| | | 2014 |
| One Shot ™ Stbl3 ™ Chemically Competent E. coli | Thermo Fisher | C737303 |

Biological samples

| | | |
|---|---|---|
| Human GBM tumor tissue | Northwestern Nervous | N/A |
| | System Tumor Bank | |
| Glioma PDX-MES83 | Gifted by Ichiro Nakano | N/A |
| | (University of Alabama). | |
| Glioma Patient-derived xenografts (PDX) | Mayo Clinic (Scottsdale, | https://www.mayo.edu/ |
| | AZ, USA) | |

Chemicals, peptides, and recombinant proteins

| | | |
|---|---|---|
| Lipofectamine ™ 2000 Transfection Reagent | Thermo Fisher | 11668019 |
| Corning ™ Matrigel ™ GFR Membrane Matrix | Fisher Scientific | 356231 |
| CellTiter-Glo ® Luminescent Cell Viability Assay | Promega | G7572 |
| Blasticidine S hydrochloride | Sigma-Aldrich | 15205-25MG |
| Polybrene Infection/Transfection Reagent | Sigma-Aldrich | TR-1003-G |
| Paclitaxel | Sigma-Aldrich | T7191-1MG |
| Takara Ex taq 250U | Clontech | RR001 |
| iQ SYBR green Supermix | Biorad | 1708884 |
| Script ™ CDNA Synthesis Kit | Biorad | 1708841 |

Experimental models: Cell lines

| | | |
|---|---|---|
| H4 | ATCC | HTB-148 ™ |
| 8-MG-BA | DSMZ-German Collection | ACC 432 |
| | of Microorganisms and | |
| | Cell Cultures GmbH | |
| KS-1, GB-1, AM-38 | JCRB Cell Bank and | IFO50436, |
| | Sekisui XenoTech, LLC | IFO50489, |
| | | IFO50492 |
| MDA-MB-468 | Gifted by Dr. Dai Horuichi | N/A |
| | (Northwestern University). | |
| MDA-MB-231, BT549, Hs578T and HCC1937 | Gifted by Dr. Maciej | N/A |
| | Lesniak (Northwestern | |
| | University) | |

Experimental models: Organisms/strains

| | | |
|---|---|---|
| Nude Mice | Charles River Laboratories | N/A |

Oligonucleotides

| | | |
|---|---|---|
| Guides for single gene CRISPR KO (Human)-See | N/A | N/A |
| Table S3 | | |
| Primers for quantitative RT-PCR (Human)-See | N/A | N/A |
| Table S4 | | |

Software, algorithms, clinical and sequencing data

| | | |
|---|---|---|
| ImageJ | Schneider et al., 2012 | https://imagej.nih.gov/ij |
| CRISPRAnalyzeR | Winter, J et al., 2017 | http://crispr-analyzer.dkfz.de |
| UCSC Xena | Goldman et al., 2020 | https://xena.ucsc.edu/ |
| Sheet 1-PTX screen raw guide counts | N/A | N/A |
| Sheet 2-Analysis of PTX screen by DESeq and | N/A | N/A |
| sgRSEA | | |
| Sheet 3-Clinical data of the patients (FIG. 4C) | N/A | N/A |
| Sheet 4-Clinical data of the patients (FIG. 4E) | N/A | N/A |

### Human GBM Patient Tissue Samples

Institutional review board approval was acquired for the samples used in this study. All patients provided written consent. Tumor samples used in this study were collected by the Brain Tumor Bank staff and clinical pathology cores that have standard operating procedures (SOPs) in which tissue is fixed and catalogued in a timely fashion.

### Animal Studies

All animal studies were performed in accordance with Northwestern's Institutional Animal Care and Usage Committee. Mice were housed in pathogen free conditions at a relatively constant temperature of 24° C. and humidity of 30-50%. Six to eight week old male and female athymic nude mice purchased from Charles River Laboratories were used in these studies.

### Intracranial Patient Derived Xenograft Mouse Model

The intracranial injection protocol was approved by The Institutional Committee on Animal Use at Northwestern University. The protocol followed to generate intracranial models is as described previously (Zhang et al., 2020). 5000 MES83, 5000 GBM12, 10⁵ GBM6/GBM6 VBB/GBM6 SSR3 O/E cells and 2×10⁵ TS543 cells were used to develop orthotopic tumors for the listed cell lines. Typically for every intracranial injection, 2.5 μl of cell suspension was prepared in sterile PBS and was loaded into a 29G Hamilton Syringe. Injection was done slowly over a period of three minutes into the left hemisphere of the mouse brain at 3 mm depth through the transcranial burr hole created 3 mm lateral and 2 mm caudal relative to midline and bregma sutures. Following injection incision was closed using 9 mm stainless steel wound clips and mouse was placed into a clean cage placed onto a heating pad until recovery from anesthesia. Mice were monitored over the period of study and were euthanized when they approached the end point as described in the IACUC protocol.

### Mammary Fat Pad Injections

The mammary fat pad injection protocol was approved by The Institutional Committee on Animal Use at Northwestern University. For mammary gland injections, 2 million MDA-MB-468 NTC (vector control) and SSR3 KO cells were diluted 1:1 with Matrigel Matrix (BD Biosciences) at a final volume of 50 μl and injected in the inguinal mammary fat pad of nude female mice. Four weeks after tumor cell injection, when the tumors reached 62.5 mm³, the mice were randomly divided into four treatment groups: (1) NTC control (2) NTC ABX (10 mg/kg) (3) SSR3 KO control (4) SSR3 KO ABX (10 mg/kg). ABX was administered by intraperitoneal injection as a single dose of 10 mg/kg and the control group were administered equal volume of PBS (diluent).

### PTX CRISPR Screen

To perform the whole genome KO CRISPR screen, we used the Brunello Library that contains 70,000 sgRNA at the coverage of 3-4 gRNA/gene plus 10,000 gRNA non-targeting controls. The library preparation, virus production and multiplicity of infection (MOI) determination was done as described in (Joung et al., 2017). We used 50 million of selected cells for the extraction of genomic DNA (Day 0). 50 million cells each were treated with PTX at concentration of 0.025 μM or DMSO control. On the 14th day and 21st day respectively the cells were harvested, the gDNA was extracted (with the Zymo Research Quick-DNA midiprep plus kit (Cat No: D4075)), and the gRNA was amplified with unique barcode primer.

For the next generation sequencing, the gRNAs were pooled together and sequenced in a Next generation sequencer (Next Seq). The samples were sequenced according to the Illumina user manual with 80 cycles of read 1 (forward) and 8 cycles of index 114. 20% PhiX were added on the Nextseq to improve library diversity and aiming for a coverage of >1000 reads per SgRNA in the library. The CRISPR screen data analysis. was performed with the bioinformatics tool CRISPR Analyzer45 (Winter et al., 2017). Deseq and sgRSEA algorithms were used to identify enriched genes. Genes/guides with raw read counts <40 were not included in the shortlisted enrichment list despite the p values. For gene ontology analysis DAVID42, 43 were used (Dennis et al., 2003).

### Generation of Single Gene Knockouts and SSR3 Overexpression Clones

Single gene knockout clones were generated in lentiCRISPRv2 (one vector system): The vector backbone was purchased from Addgene and the protocol for guide cloning and generation of virus was as described in (Sanjana et al., 2014; Shalem et al., 2014). The guides for single gene CRISPR knockout are listed in Table 3. The single gene knockout for all the 7 genes was tested at transcript level using real time PCR. The primers for real time PCR are listed in Table 4. For SSR3 overexpression, SSR3 ORF was ordered from Dharmacon (Horizon) as CCSB-Broad Lentiviral Expression Collection (Catalog ID: OHS6085-213574251).

### Cell Viability Assay

GBM cells were seeded at a density of 5000 cells per well in a 96 well plate. One day after seeding, cells were checked for attachment and confluence (60-70%). Media was removed from wells and 100 μl fresh media with PTX dissolved in DMSO or PTX ranging in concentrations from 0.0005 μM to 0.5 μM was placed into wells. 72 hours later cell viability was determined by CellTiter Glo (Promega).

### Immunofluorescence Staining

H4 SSR3 knockout and non-targeting control cells were grown on glass coverslips. After 48 h of treatment with PTX or DMSO the coverslips were fixed in methanol for 5 minutes. The cells were then stained with α-tubulin and SSR3 antibodies at 1:100 dilution and the images were acquired on the Nikon A1 (C) confocal microscope. Microtubule bundles per cell cluster were counted for the analysis.

### Immunohistochemistry (IHC) Staining

Immunohistochemistry and H&E staining was performed using standard immunoperoxidase staining on formalin-fixed paraffin-embedded tissue sections of 5 μm thick from resected recurrent tumors. Mouse and human sections were stained with anti-SSR3 monoclonal antibodies. The procedure was performed on a DAKO Autostainer Link 48 slide stainer (Agilent Technologies). Sections were counterstained with hematoxylin, dehydrated, and mounted with coverslips. The slides were scanned and digitalized with the Hamamatsu K.K. Nanozoomer 2.0 HT and were visualized with the NDP.view2 Viewing software. A board-certified neuropathologist evaluated the staining digitally to ensure the appropriate quality of the tumor tissue.

### IHC Image Analysis

For the quantification of the IHC images for SSR3, a neuropathologist outlined the tumoral regions on each sample in a blinded fashion regarding treatments, survival days, and other clinical characteristics. We used HistoQuest version 6.0 software (TissueGnostics) to generate quantitative measurements of the intensity of SSR3.

Development of Explant Cultures from Fresh GBM Tumor Tissues

Freshly resected GBM tumor tissue piece was chopped into smaller size pieces using sterile scalpel blades. The tumor pieces were then transferred to 50 ml tubes and were subjected to collagenase treatment at 37° C. for 1 h. Every 15 minutes the tube was mixed well. The collagenase digested tumor suspension was passed through 70 μm cell strainer. The filtrate was then centrifuged at 1000 rpm for 5 mins and the pellet was seeded in a T25 flask. Media was replaced after 24 h. The glioma cells that were developed were immediately (within first 2 passages) characterized for PTX susceptibility.

### Quantification and Statistical Analysis

Cox Proportional-Hazards Model (Cox) was used to identify clinically relevant genes from the CRISPR shortlisted genes using the TCGA breast cancer dataset. We performed two different analyses with gene expression and a treatment indicator as independent variables. The regressions included interaction terms between the treatment indicator and gene expression. We used two different models, one with a treatment indicator for untreated vs. taxane treated patients and another where the indicator was for taxane treated vs. non-taxane treated patients. Further, bootstrap analysis was used to assess the method for selecting the 51 candidate genes. In particular, we were interested in testing for the null hypothesis that the 51 candidate list was a random subset of genes. To test this, we computed the probability of finding nine or more significant genes from a random list of 51 candidates: we sampled 51 genes at random from a list of 64,000 from the human genome, carried out the Cox analysis for the two models described above, then we recorded the number of significant genes at the 0.05 level. This process was repeated 10,000 times.

All other statistical analyses were performed using Graphpad Software (Prism). In-vitro dose response curves were generated by fitting experimental cell viability data to a sigmoidal curve. Differences in dose response was evaluated through an extra sum of squares test. Two-tailed student's t-test or one-way ANOVA was used to measure statistical differences between two groups or greater than two groups respectively. Statistical analysis of animal survival was performed using a log-rank test. Pearson correlation coefficient r was determined to measure the strength of correlation. Image J analysis (Schneider et al., 2012) was used to perform densitometry analysis on the western blots.

### REFERENCES

Acosta-Alvear, D., Karagoz, G. E., Frohlich, F., Li, H., Walther, T. C., and Walter, P. (2018). The unfolded protein response and endoplasmic reticulum protein targeting machineries converge on the stress sensor IRE1. Elife 7.
Carpentier, A., Canney, M., Vignot, A., Reina, V., Beccaria, K., Horodyckid, C., Karachi, C., Leclercq, D., Lafon, C., Chapelon, J. Y., et al. (2016). Clinical trial of blood-brain barrier disruption by pulsed ultrasound. Sci Transl Med 8, 343re342.
Chamberlain, M. C., and Kormanik, P. (1995). Salvage chemotherapy with paclitaxel for recurrent primary brain tumors. J Clin Oncol 13, 2066-2071.
Chang, S. M., Kuhn, J. G., Rizzo, J., Robins, H. I., Schold, S. C., Jr., Spence, A. M., Berger, M. S., Mehta, M. P., Bozik, M. E., Pollack, I., et al. (1998). Phase I study of paclitaxel in patients with recurrent malignant glioma: a North American Brain Tumor Consortium report. J Clin Oncol 16, 2188-2194.
Dennis, G., Jr., Sherman, B. T., Hosack, D. A., Yang, J., Gao, W., Lane, H. C., and Lempicki, R. A. (2003). DAVID: Database for Annotation, Visualization, and Integrated Discovery. Genome Biol 4, P3.
Drappatz, J., Brenner, A., Wong, E. T., Eichler, A., Schiff, D., Groves, M. D., Mikkelsen, T., Rosenfeld, S., Sarantopoulos, J., Meyers, C. A., et al. (2013). Phase I study of GRN1005 in recurrent malignant glioma. Clin Cancer Res 19, 1567-1576.
Fellner, S., Bauer, B., Miller, D. S., Schaffrik, M., Fankhanel, M., Spruss, T., Bernhardt, G., Graeff, C., Farber, L., Gschaidmeier, H., et al. (2002). Transport of paclitaxel (Taxol) across the blood-brain barrier in vitro and in vivo. J Clin Invest 110, 1309-1318.
Fetell, M. R., Grossman, S. A., Fisher, J. D., Erlanger, B., Rowinsky, E., Stockel, J., and Piantadosi, S. (1997). Preirradiation paclitaxel in glioblastoma multiforme: efficacy, pharmacology, and drug interactions. New Approaches to Brain Tumor Therapy Central Nervous System Consortium. J Clin Oncol 15, 3121-3128.
Gabikian, P., Tyler, B. M., Zhang, I., Li, K. W., Brem, H., and Walter, K. A. (2014). Radiosensitization of malignant gliomas following intracranial delivery of paclitaxel biodegradable polymer microspheres. J Neurosurg 120, 1078-1085.
Gargini, R., Segura-Collar, B., Herranz, B., Garcia-Escudero, V., Romero-Bravo, A., Nunez, F. J., Garcia-Perez, D., Gutierrez-Guaman, J., Ayuso-Sacido, A., Seoane, J., et al. (2020). The IDH-TAU-EGFR triad defines the neovascular landscape of diffuse gliomas. Sci Transl Med 12.
Gradishar, W. J., Tjulandin, S., Davidson, N., Shaw, H., Desai, N., Bhar, P., Hawkins, M., and O'Shaughnessy, J. (2005). Phase III trial of nanoparticle albumin-bound paclitaxel compared with polyethylated castor oil-based paclitaxel in women with breast cancer. J Clin Oncol 23, 7794-7803.
Hatzis, C., Pusztai, L., Valero, V., Booser, D. J., Esserman, L., Lluch, A., Vidaurre, T., Holmes, F., Souchon, E., Wang, H., et al. (2011). A genomic predictor of response and survival following taxane-anthracycline chemotherapy for invasive breast cancer. JAMA 305, 1873-1881.
Heimans, J. J., Taphoorn, M. J., Vermorken, J. B., Eeltink, C., Wolbers, J. G., Meijer, O. W., and Beijnen, J. H. (1995). Does paclitaxel penetrate into brain tumor tissue? J Natl Cancer Inst 87, 1804-1805.
Husemann, L. C., Reese, A., Radine, C., Piekorz, R. P., Budach, W., Sohn, D., and Janicke, R. U. (2020). The microtubule targeting agents eribulin and paclitaxel activate similar signaling pathways and induce cell death predominantly in a caspase-independent manner. Cell Cycle 19, 464-478.
Idbaih, A., Canney, M., Belin, L., Desseaux, C., Vignot, A., Bouchoux, G., Asquier, N., Law-Ye, B., Leclercq, D., Bissery, A., et al. (2019). Safety and Feasibility of Repeated and Transient Blood-Brain Barrier Disruption by Pulsed Ultrasound in Patients with Recurrent Glioblastoma. Clin Cancer Res 25, 3793-3801.
Itoh, M., Iwamoto, T., Matsuoka, J., Nogami, T., Motoki, T., Shien, T., Taira, N., Niikura, N., Hayashi, N., Ohtani, S., et al. (2014). Estrogen receptor (ER) mRNA expression and molecular subtype distribution in ER-negative/progesterone receptor-positive breast cancers. Breast Cancer Res Treat 143, 403-409.
Janssen, K., Pohlmann, S., Janicke, R. U., Schulze-Osthoff, K., and Fischer, U. (2007). Apaf-1 and caspase-9 deficiency prevents apoptosis in a Bax-controlled pathway and promotes clonogenic survival during paclitaxel treatment. Blood 110, 3662-3672.
Joung, J., Konermann, S., Gootenberg, J. S., Abudayyeh, O. O., Platt, R. J., Brigham, M. D., Sanjana, N. E., and Zhang, F. (2017). Genome-scale CRISPR-Cas9 knockout and transcriptional activation screening. Nat Protoc 12, 828-863
Juul, N., Szallasi, Z., Eklund, A. C., Li, Q., Burrell, R. A., Gerlinger, M., Valero, V., Andreopoulou, E., Esteva, F. J., Symmans, W. F., et al. (2010). Assessment of an RNA interference screen-derived mitotic and ceramide pathway metagene as a predictor of response to neoadjuvant paclitaxel for primary triple-negative breast cancer: a retrospective analysis of five clinical trials. Lancet Oncol 11, 358-365.
Koziara, J. M., Lockman, P. R., Allen, D. D., and Mumper, R. J. (2004). Paclitaxel nanoparticles for the potential treatment of brain tumors. J Control Release 99, 259-269.
Kumthekar, P., Tang, S. C., Brenner, A. J., Kesari, S., Piccioni, D. E., Anders, C., Carrillo, J., Chalasani, P., Kabos, P., Puhalla, S., et al. (2020). ANG1005, a Brain-Penetrating Peptide-Drug Conjugate, Shows Activity in Patients with Breast Cancer with Leptomeningeal Carcinomatosis and Recurrent Brain Metastases. Clin Cancer Res 26, 2789-2799.
Lanni, J. S., Lowe, S. W., Licitra, E. J., Liu, J. O., and Jacks, T. (1997). p53-independent apoptosis induced by paclitaxel through an indirect mechanism. Proc Natl Acad Sci USA 94, 9679-9683.
Lesser, G. J., Grossman, S. A., Eller, S., and Rowinsky, E. K. (1995). The distribution of systemically administered [3H]-paclitaxel in rats: a quantitative autoradiographic study. Cancer Chemother Pharmacol 37, 173-178.
Lidar, Z., Mardor, Y., Jonas, T., Pfeffer, R., Faibel, M., Nass, D., Hadani, M., and Ram, Z. (2004). Convection-enhanced delivery of paclitaxel for the treatment of recurrent malignant glioma: a phase I/II clinical study. J Neurosurg 100, 472-479.
Maire, V., Baldeyron, C., Richardson, M., Tesson, B., Vincent-Salomon, A., Gravier, E., Marty-Prouvost, B., De Koning, L., Rigaill, G., Dumont, A., et al. (2013a). TTK/hMPSI is an attractive therapeutic target for triple-negative breast cancer. PLOS One 8, e63712.
Maire, V., Nemati, F., Richardson, M., Vincent-Salomon, A., Tesson, B., Rigaill, G., Gravier, E., Marty-Prouvost, B., De Koning, L., Lang, G., et al. (2013b). Polo-like kinase 1: a potential therapeutic option in combination with conventional chemotherapy for the management of patients with triple-negative breast cancer. Cancer Res 73, 813-823.
Markman, M., Liu, P. Y., Wilczynski, S., Monk, B., Copeland, L. J., Alvarez, R. D., Jiang, C., Alberts, D., Southwest Oncology, G., and Gynecologic Oncology, G. (2003). Phase III randomized trial of 12 versus 3 months of maintenance paclitaxel in patients with advanced ovarian cancer after complete response to platinum and paclitaxel-based chemotherapy: a Southwest Oncology Group and Gynecologic Oncology Group trial. J Clin Oncol 21, 2460-2465
Maubant, S., Tesson, B., Maire, V., Ye, M., Rigaill, G., Gentien, D., Cruzalegui, F., Tucker, G. C., Roman-Roman, S., and Dubois, T. (2015). Transcriptome analysis of Wnt3a-treated triple-negative breast cancer cells. PLOS One 10, e0122333.
Mielgo, A., Torres, V. A., Clair, K., Barbero, S., and Stupack, D. G. (2009). Paclitaxel promotes a caspase 8-mediated apoptosis through death effector domain association with microtubules. Oncogene 28, 3551-3562.
Njiaju, U. O., Gamazon, E. R., Gorsic, L. K., Delaney, S. M., Wheeler, H. E., Im, H. K., and Dolan, M. E. (2012). Whole-genome studies identify solute carrier transporters in cellular susceptibility to paclitaxel. Pharmacogenet Genomics 22, 498-507.
Oh, J., Sahgal, A., Sanghera, P., Tsao, M. N., Davey, P., Lam, K., Symons, S., Aviv, R., and Perry, J. R. (2011). Glioblastoma: patterns of recurrence and efficacy of salvage treatments. Can J Neurol Sci 38, 621-625.
Postma, T. J., Heimans, J. J., Luykx, S. A., van Groeningen, C. J., Beenen, L. F., Hoekstra, O. S., Taphoorn, M. J., Zonnenberg, B. A., Klein, M., and Vermorken, J. B. (2000). A phase II study of paclitaxel in chemonaive patients with recurrent high-grade glioma. Ann Oncol 11, 409-413.
Prados, M. D., Schold, S. C., Spence, A. M., Berger, M. S., McAllister, L. D., Mehta, M. P., Gilbert, M. R., Fulton, D., Kuhn, J., Lamborn, K., et al. (1996). Phase II study of paclitaxel in patients with recurrent malignant glioma. J Clin Oncol 14, 2316-2321.
Pusztai, L., Jeong, J. H., Gong, Y., Ross, J. S., Kim, C., Paik, S., Rouzier, R., Andre, F., Hortobagyi, G. N., Wolmark, N., and Symmans, W. F. (2009). Evaluation of microtubule-associated protein-Tau expression as a prognostic and predictive marker in the NSABP-B 28 randomized clinical trial. J Clin Oncol 27, 4287-4292.
Rapp, M., Baernreuther, J., Turowski, B., Steiger, H. J., Sabel, M., and Kamp, M. A. (2017). Recurrence Pattern Analysis of Primary Glioblastoma. World Neurosurg 103, 733-740.
Rosell, R., Gatzemeier, U., Betticher, D. C., Keppler, U., Macha, H. N., Pirker, R., Berthet, P., Breau, J. L., Lianes, P., Nicholson, M., et al. (2002). Phase III randomised trial comparing paclitaxel/carboplatin with paclitaxel/cisplatin in patients with advanced non-small-cell lung cancer: a cooperative multinational trial. Ann Oncol 13, 1539-1549.
Rouzier, R., Rajan, R., Wagner, P., Hess, K. R., Gold, D. L., Stec, J., Ayers, M., Ross, J. S., Zhang, P., Buchholz, T. A., et al. (2005). Microtubule-associated protein tau: a marker of paclitaxel sensitivity in breast cancer. Proc Natl Acad Sci USA 102, 8315-8320.
Russell, P., Hennessy, B. T., Li, J., Carey, M. S., Bast, R. C., Freeman, T., and Venkitaraman, A. R. (2012). Cyclin G1 regulates the outcome of taxane-induced mitotic checkpoint arrest. Oncogene 31, 2450-2460.
Sanjana, N. E., Shalem, O., and Zhang, F. (2014). Improved vectors and genome-wide libraries for CRISPR screening. Nat Methods 11, 783-784.
Shalem, O., Sanjana, N. E., Hartenian, E., Shi, X., Scott, D. A., Mikkelson, T., Heckl, D., Ebert, B. L., Root, D. E., Doench, J. G., and Zhang, F. (2014). Genome-scale CRISPR-Cas9 knockout screening in human cells. Science 343, 84-87.
Shen, Y., Pi, Z., Yan, F., Yeh, C. K., Zeng, X., Diao, X., Hu, Y., Chen, S., Chen, X., and Zheng, H. (2017). Enhanced delivery of paclitaxel liposomes using focused ultrasound with microbubbles for treating nude mice bearing intracranial glioblastoma xenografts. Int J Nanomedicine 12, 5613-5629.
Shimada, K., Matsuyoshi, S., Nakamura, M., Ishida, E., Kishi, M., and Konishi, N. (2004). Phosphorylation of FADD is critical for sensitivity to anticancer drug-induced apoptosis. Carcinogenesis 25, 1089-1097.
Sonabend, A. M., and Stupp, R. (2019). Overcoming the Blood-Brain Barrier with an Implantable Ultrasound Device. Clin Cancer Res 25, 3750-3752.
Stupp, R., Taillibert, S., Kanner, A., Read, W., Steinberg, D., Lhermitte, B., Toms, S., Idbaih, A., Ahluwalia, M. S., Fink, K., et al. (2017). Effect of Tumor-Treating Fields Plus Maintenance Temozolomide vs Maintenance Temozolomide Alone on Survival in Patients With Glioblastoma: A Randomized Clinical Trial. JAMA 318, 2306-2316.
Swanton, C., Marani, M., Pardo, O., Warne, P. H., Kelly, G., Sahai, E., Elustondo, F., Chang, J., Temple, J., Ahmed, A. A., et al. (2007). Regulators of mitotic arrest and ceramide metabolism are determinants of sensitivity to paclitaxel and other chemotherapeutic drugs. Cancer Cell 11, 498-512.
Von Hoff, D. D., Ervin, T., Arena, F. P., Chiorean, E. G., Infante, J., Moore, M., Seay, T., Tjulandin, S. A., Ma, W. W., Saleh, M. N., et al. (2013). Increased survival in pancreatic cancer with nab-paclitaxel plus gemcitabine. N Engl J Med 369, 1691-1703.
Walter, K. A., Cahan, M. A., Gur, A., Tyler, B., Hilton, J., Colvin, O. M., Burger, P. C., Domb, A., and Brem, H. (1994). Interstitial taxol delivered from a biodegradable polymer implant against experimental malignant glioma. Cancer Res 54, 2207-2212.
Weaver, B. A. (2014). How Taxol/paclitaxel kills cancer cells. Mol Biol Cell 25, 2677-2681.
Wertz, I. E., Kusam, S., Lam, C., Okamoto, T., Sandoval, W., Anderson, D. J., Helgason, E., Ernst, J. A., Eby, M., Liu, J., et al. (2011). Sensitivity to antitubulin chemotherapeutics is regulated by MCL1 and FBW7. Nature 471, 110-114.
Whitehurst, A. W., Bodemann, B. O., Cardenas, J., Ferguson, D., Girard, L., Peyton, M., Minna, J. D., Michnoff, C., Hao, W., Roth, M. G., et al. (2007). Synthetic lethal screen identification of chemosensitizer loci in cancer cells. Nature 446, 815-819.
Wick, W., Stupp, R., Beule, A. C., Bromberg, J., Wick, A., Ernemann, U., Platten, M., Marosi, C., Mason, W. P., van den Bent, M., et al. (2008). A novel tool to analyze MRI recurrence patterns in glioblastoma. Neuro Oncol 10, 1019-1024.
Winter, J., Schwering, M., Pelz, O., Rauscher, B., Zhan, T., Heigwer, F., and Boutros, M. (2017). CRISPRAnalyzeR: Interactive analysis, annotation and documentation of pooled CRISPR screens. 109967.
Yang, W., Soares, J., Greninger, P., Edelman, E. J., Lightfoot, H., Forbes, S., Bindal, N., Beare, D., Smith, J. A., Thompson, I. R., et al. (2013). Genomics of Drug Sensitivity in Cancer (GDSC): a resource for therapeutic biomarker discovery in cancer cells. Nucleic Acids Res 41, D955-961.
Zhang, D. Y., Dmello, C., Chen, L., Arrieta, V. A., Gonzalez-Buendia, E., Kane, J. R., Magnusson, L. P., Baran, A., James, C. D., Horbinski, C., et al. (2020). Ultrasound-mediated Delivery of Paclitaxel for Glioma: A Comparative Study of Distribution, Toxicity, and Efficacy of Albumin-bound Versus Cremophor Formulations. Clin Cancer Res 26, 477-486.
Fons, R. D., Bogert, B. A., and Hegde, R. S. (2003). Substrate-specific function of the translocon-associated protein complex during translocation across the ER membrane. J Cell Biol 160, 529-539.
Goldman, M. J., Craft, B., Hastie, M., Repecka, K., McDade, F., Kamath, A., Banerjee, A., Luo, Y., Rogers, D., Brooks, A. N., et al. (2020). Visualizing and interpreting cancer genomics data via the Xena platform. Nat Biotechnol 38, 675-678.
Gorlich, D., Hartmann, E., Prehn, S., and Rapoport, T. A. (1992). A protein of the endoplasmic reticulum involved early in polypeptide translocation. Nature 357, 47-52.
Hartmann, E., Gorlich, D., Kostka, S., Otto, A., Kraft, R., Knespel, S., Burger, E., Rapoport, T. A., and Prehn, S. (1993). A tetrameric complex of membrane proteins in the endoplasmic reticulum. Eur J Biochem 214, 375-381.
Iwawaki, T., Akai, R., Yamanaka, S., and Kohno, K. (2009). Function of IRE1 alpha in the placenta is essential for placental development and embryonic viability. Proc Natl Acad Sci USA 106, 16657-16662.
Lerose, R., Musto, P., Aieta, M., Papa, C., and Tartarone, A. (2012). Off-label use of anti-cancer drugs between clinical practice and research: the Italian experience. Eur J Clin Pharmacol 68, 505-512.
Nagasawa, K., Higashi, T., Hosokawa, N., Kaufman, R. J., and Nagata, K. (2007). Simultaneous induction of the four subunits of the TRAP complex by ER stress accelerates ER degradation. EMBO Rep 8, 483-489.
Schneider, C. A., Rasband, W. S., and Eliceiri, K. W. (2012). NIH Image to ImageJ: 25 years of image analysis. Nat Methods 9, 671-675.
Yamaguchi, Y. L., Tanaka, S. S., Oshima, N., Kiyonari, H., Asashima, M., and Nishinakamura, R. (2011). Translocon-associated protein subunit Trap-gamma/Ssr3 is required for vascular network formation in the mouse placenta. Dev Dyn 240, 394-403.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

1. A method comprising:
(a) detecting expression of signal sequence receptor 3 (SSR3) in a cancer sample from a subject; and optionally
(b) administering a tubulin inhibitor to the subject.

2. The method of claim 1, wherein if the detected expression of SSR3 in the cancer sample is greater than a defined baseline level, the subject is administered the tubulin inhibitor.

3. The method of claim 1, wherein the cancer sample is obtained from a biopsy of a solid tumor from the subject.

4. The method of claim 1, wherein the cancer sample is glioblastoma or breast cancer.

5. (canceled)

6. The method of claim 1, wherein the tubulin inhibitor is a taxane.

7. (canceled)

8. The method of claim 1, wherein the tubulin inhibitor is a vinca alkaloid.

9. (canceled)

10. The method of claim 1, wherein the subject has glioblastoma and the method comprises administering the tubulin inhibitor to the glioblastoma via transient blood brain barrier (BBB) opening.

11. The method of claim 10, wherein the transient BBB opening is achieved via administering ultrasound treatment and concomitantly administering microbubbles.

12. The method of claim 10, wherein the tubulin inhibitor is abraxane and the abraxane is delivered to the glioblastoma at a concentration of at least about 0.3-0.5 μM.

13-19. (canceled)

20. A method comprising:
(a) detecting expression of one or more biomarkers in a cancer sample from a subject, wherein the biomarker is selected from the group consisting of: signal sequence receptor 3 (SSR3), interleukin-1 receptor-associated kinase 4 (IRAK4), transmembrane protein 131 (TMEM131), enhancer of polycomb homolog 2 (EPC2), muscleblind like splicing regulator 1 (MBNL1), zinc finger protein 813 (ZNF813), zinc finger and BTB domain containing 20 (ZBTB20); and optionally
(b) administering a tubulin inhibitor to the subject.

21. The method of claim 20, wherein if the detected expression of the biomarker in the cancer sample is greater than a defined baseline level, the subject is administered the tubulin inhibitor.

22. (canceled)

23. The method of claim 20, wherein the cancer sample is glioblastoma or breast cancer.

24. (canceled)

25. The method of claim 20, wherein the tubulin inhibitor is a taxane or a vinca alkaloid.

26-28. (canceled)

29. The method of claim 20, wherein the subject has glioblastoma and the method comprises administering the tubulin inhibitor to the glioblastoma via transient blood brain barrier (BBB) opening.

30. The method of claim 29, wherein the transient BBB opening is achieved via administering ultrasound treatment and concomitantly administering microbubbles.

31. The method of claim 29, wherein the tubulin inhibitor is abraxane and the abraxane is delivered to the glioblastoma at a concentration of at least about 0.3-0.5 μM.

32. A method for identifying a cancer that is susceptible to treatment with a tubulin inhibitor, the method comprising:
(a) detecting expression of one or more biomarkers selected from the group consisting of: signal sequence receptor 3 (SSR3), interleukin-1 receptor-associated kinase 4 (IRAK4), transmembrane protein 131 (TMEM131), enhancer of polycomb homolog 2 (EPC2), muscleblind like splicing regulator 1 (MBNL1), zinc finger protein 813 (ZNF813), zinc finger and BTB domain containing 20 (ZBTB20), in a cancer sample, and if the detected expression of the one or more biomarkers in the cancer sample is greater than a defined baseline level, then
(b) identifying the cancer as susceptible to treatment with a tubulin inhibitor.

33. The method of claim 32, wherein the cancer is a glioblastoma or breast cancer.

34. (canceled)

35. The method of claim 32, wherein the tubulin inhibitor is a taxane or a vinca alkaloid.

36-39. (canceled)

40. The method of claim 32, wherein the biomarker is SSR3.


SEQUENCE LISTING (55 sequences)

SEQ ID NO: 1 - DNA, 25 bp, Artificial Sequence
  Synthetic- guide for single gene CRISPR KO- ERN1 1F
caccggagtcctcgccatgccggcc

SEQ ID NO: 2 - DNA, 25 bp, Artificial Sequence
  Synthetic- guide for single gene CRISPR KO- ERN1 1R
aaacggccggcatggcgaggactcc

SEQ ID NO: 3 - DNA, 25 bp, Artificial Sequence
  Synthetic- guide for single gene CRISPR KO- ERN1 2F
caccgcttgttgtttgtgtcaacgc

SEQ ID NO: 4 - DNA, 25 bp, Artificial Sequence
  Synthetic- guide for single gene CRISPR KO- ERN1 2R
aaacgcgttgacacaaacaacaagc

SEQ ID NO: 5 - DNA, 25 bp, Artificial Sequence
  Synthetic- guide for single gene CRISPR KO- SSR3 1F
caccgaggacttggccgagagattg

SEQ ID NO: 6 - DNA, 25 bp, Artificial Sequence
  Synthetic- guide for single gene CRISPR KO- SSR3 1R
aaaccaatctctcggccaagtcctc

SEQ ID NO: 7 - DNA, 25 bp, Artificial Sequence
  Synthetic- guide for single gene CRISPR KO- SSR3 2F
caccgctccaaacagcagtctgagg

SEQ ID NO: 8 - DNA, 25 bp, Artificial Sequence
  Synthetic- guide for single gene CRISPR KO- SSR3 2R
aaaccctcagactgctgtttggagc

SEQ ID NO: 9 - DNA, 25 bp, Artificial Sequence
  Synthetic- guide for single gene CRISPR KO- IRAK4 1F
caccgcctgggagcaaaagactcgc

SEQ ID NO: 10 - DNA, 25 bp, Artificial Sequence
  Synthetic- guide for single gene CRISPR KO- IRAK4 1R
aaacgcgagtcttttgctcccaggc

SEQ ID NO: 11 - DNA, 25 bp, Artificial Sequence
  Synthetic- guide for single gene CRISPR KO IRAK4 2F
caccgctcatgtgccaagaaagtgg

SEQ ID NO: 12 - DNA, 25 bp, Artificial Sequence
  Synthetic- guide for single gene CRISPR KO- IRAK4 2R
aaacccactttcttggcacatgagc

SEQ ID NO: 13 - DNA, 25 bp, Artificial Sequence
  Synthetic- guide for single gene CRISPR KO- TMEM131 1F
caccgcttccatacttagctcaccc

SEQ ID NO: 14 - DNA, 25 bp, Artificial Sequence
  Synthetic- guide for single gene CRISPR KO- TMEM131 1R
aaacgggtgagctaagtatggaagc

SEQ ID NO: 15 - DNA, 25 bp, Artificial Sequence
  Synthetic- guide for single gene CRISPR KO- TMEM131 2F
caccggaatatgggagccatttcga

SEQ ID NO: 16 - DNA, 25 bp, Artificial Sequence
  Synthetic- guide for single gene CRISPR KO- TMEM131 2R
aaactcgaaatggctcccatattcc

SEQ ID NO: 17 - DNA, 25 bp, Artificial Sequence
  Synthetic- guide for single gene CRISPR KO- EPC2 1F
caccggatgtataggatttgcaagg

SEQ ID NO: 18 - DNA, 25 bp, Artificial Sequence
  Synthetic- guide for single gene CRISPR KO- EPC2 1R
aaacccttgcaaatcctatacatcc

SEQ ID NO: 19 - DNA, 25 bp, Artificial Sequence
  Synthetic- guide for single gene CRISPR KO- EPC2 2F
caccgtattacaatcgcttgtacaa

SEQ ID NO: 20 - DNA, 25 bp, Artificial Sequence
  Synthetic- guide for single gene CRISPR KO- EPC2 2R
aaacttgtacaagcgattgtaatac

SEQ ID NO: 21 - DNA, 25 bp, Artificial Sequence
  Synthetic- guide for single gene CRISPR KO- MBNL1 1F
caccggtatgtagagagttccagag

SEQ ID NO: 22 - DNA, 25 bp, Artificial Sequence
  Synthetic- guide for single gene CRISPR KO- MBNL1 1R
aaacctctggaactctctacatacc

SEQ ID NO: 23 - DNA, 25 bp, Artificial Sequence
  Synthetic- guide for single gene CRISPR KO- MBNL1 2F
caccggtatgtcgagagtaccaacg

SEQ ID NO: 24 - DNA, 25 bp, Artificial Sequence
  Synthetic- guide for single gene CRISPR KO- MBNL1 2R
aaaccgttggtactctcgacatacc

SEQ ID NO: 25 - DNA, 25 bp, Artificial Sequence
  Synthetic- guide for single gene CRISPR KO- ZNF813 1F
caccgagacatgaaagtcatcacac

SEQ ID NO: 26 - DNA, 25 bp, Artificial Sequence
  Synthetic- guide for single gene CRISPR KO- ZNF813 1R
aaacgtgtgatgactttcatgtctc

SEQ ID NO: 27 - DNA, 25 bp, Artificial Sequence
  Synthetic- guide for single gene CRISPR KO- ZBTB20 1F
caccgcagcgacatcgagatcccgt

SEQ ID NO: 28 - DNA, 25 bp, Artificial Sequence
  Synthetic- guide for single gene CRISPR KO- ZBTB20 1R
aaacacgggatctcgatgtcgctgc

SEQ ID NO: 29 - DNA, 21 bp, Artificial Sequence
  Synthetic- primers for quantitative RT-PCR- SSR3 F
ggatttcagccgcaatctctc

SEQ ID NO: 30 - DNA, 22 bp, Artificial Sequence
  Synthetic- primers for quantitative RT-PCR- SSR3 R
tgccatattcgccagtataacc

SEQ ID NO: 31 - DNA, 21 bp, Artificial Sequence
  Synthetic- primers for quantitative RT-PCR- IRAK4 F
cctgactcctcaagtccagaa

SEQ ID NO: 32 - DNA, 23 bp, Artificial Sequence
  Synthetic- primers for quantitative RT-PCR- IRAK4 R
acagaaatgggtcgttcatcaaa

SEQ ID NO: 33 - DNA, 20 bp, Artificial Sequence
  Synthetic- primers for quantitative RT-PCR- TMEM131 F
gtgatgaccctcgtagtggc

SEQ ID NO: 34 - DNA, 20 bp, Artificial Sequence
  Synthetic- primers for quantitative RT-PCR- TMEM131 R
cggtctgtagtagccctcca

SEQ ID NO: 35 - DNA, 20 bp, Artificial Sequence
  Synthetic- primers for quantitative RT-PCR- EPC2 F
caagccgctgcctatctacc

SEQ ID NO: 36 - DNA, 23 bp, Artificial Sequence
  Synthetic- primers for quantitative RT-PCR- EPC2 R
aaatgatgttccgattcctcctc

SEQ ID NO: 37 - DNA, 23 bp, Artificial Sequence
  Synthetic- primers for quantitative RT-PCR- MBNL1 F
gctgttagtgtcacaccaattcg

SEQ ID NO: 38 - DNA, 22 bp, Artificial Sequence
  Synthetic- primers for quantitative RT-PCR- MBNL1 R
aggcgattactcgtccattttc

SEQ ID NO: 39 - DNA, 21 bp, Artificial Sequence
  Synthetic- primers for quantitative RT-PCR- ZNF813 F
acactggagactttcgctttc

SEQ ID NO: 40 - DNA, 23 bp, Artificial Sequence
  Synthetic- primers for quantitative RT-PCR- ZNF813 R
gtttcgcttccgattaaagacct

SEQ ID NO: 41 - DNA, 21 bp, Artificial Sequence
  Synthetic- primers for quantitative RT-PCR- ZBTB20 F
gacaggatctactcggcactc

SEQ ID NO: 42 - DNA, 20 bp, Artificial Sequence
  Synthetic- primers for quantitative RT-PCR- ZBTB20 R
actgcgccgctgtaaaaaga

SEQ ID NO: 43 - DNA, 20 bp, Artificial Sequence
  Synthetic- primers for quantitative RT-PCR- CEP63 F
tgggaaggacgtacacatgc

SEQ ID NO: 44 - DNA, 23 bp, Artificial Sequence
  Synthetic- primers for quantitative RT-PCR- CEP63 R
acatccaactgactcctaagact

SEQ ID NO: 45 - DNA, 19 bp, Artificial Sequence
  Synthetic- primers for quantitative RT-PCR- RPS28 F
cgatccatcatccgcaatg

SEQ ID NO: 46 - DNA, 18 bp, Artificial Sequence
  Synthetic- primers for quantitative RT-PCR- RPS28 R
agccaagctcagcgcaac

SEQ ID NO: 47 - protein, 198 aa, Homo sapiens
  MISC_FEATURE (1)..(198): SSR3 isoform 1
MAPKGSSKQQSEEDLLLQDFSRNLSAKSSALFFGNAFIVSAIPIWLYWRIWHMDLIQSAVLYSVMTLVSTYLVAFAYKNVKFVLKHKVAQKREDAVSKEVTRKLSEADNRKMSRKEKDERFDSCLKYGHHKRGILWKKNEVADYEATTFSIFYNNTLFLVVVIVASFFILKNFNPTVNYILSISASSGLIALLSTGSK

SEQ ID NO: 48 - protein, 343 aa, Homo sapiens
  MISC_FEATURE (1)..(343): MBNL1 isoform 8
MGRCSRENCKYLHPPPHLKTQLEINGRNNLIQQKNMAMLAQQMQLANAMMPGAPLQPVPMFSVAPSLATNASAAAFNPYLGPVSPSLVPAEILPTAPMLVTGNPGVPVPAAAAAAAQKLMRTDRLEVCREYQRGNCNRGENDCRFAHPADSTMIDTNDNTVTVCMDYIKGRCSREKCKYFHPPAHLQAKIKAAQYQVNQAAAAQAAATAAAMTQSAVKSLKRPLEATFDLGIPQAVLPPLPKRPALEKTNGATAVFNTGIFQYQQALANMQLQQHTAFLPPGSILCMTPATSVVPMVHGATPATVSAATTSATSVPFAATATANQIPIISAEHLTSHKYVTQM

SEQ ID NO: 49 - protein, 1176 aa, Homo sapiens
  MISC_FEATURE (1)..(1176): TDRD1 isoform 2
MSVKSPFNVMSRNNLEAPPCKMTEPFNFEKNENKLPPHESLRSPGTLPNHPNFRLKSSENGNKKNNFLLCEQTKQYLASQEDNSVSSNPNGINGEVVGSKGDRKKLPAGNSVSPPSAESNSPPKEVNIKPGNNVRPAKSKKLNKLVENSLSISNPGLFTSLGPPLRSTTCHRCGLFGSLRCSQCKQTYYCSTACQRRDWSAHSIVCRPVQPNFHKLENKSSIETKDVEVNNKSDCPLGVTKEIAIWAERIMFSDLRSLQLKKTMEIKGTVTEFKHPGDFYVQLYSSEVLEYMNQLSASLKETYANVHEKDYIPVKGEVCIAKYTVDQTWNRAIIQNVDVQQKKAHVLYIDYGNEEIIPLNRIYHLNRNIDLFPPCAIKCFVANVIPAEGNWSSDCIKATKPLLMEQYCSIKIVDILEEEVVTFAVEVELPNSGKLLDHVLIEMGYGLKPSGQDSKKENADQSDPEDVGKMTTENNIVVDKSDLIPKVLTLNVGDEFCGVVAHIQTPEDFFCQQLQSGRKLAELQASLSKYCDQLPPRSDFYPAIGDICCAQFSEDDQWYRASVLAYASEESVLVGYVDYGNFEILSLMRLCPIIPKLLELPMQAIKCVLAGVKPSLGIWTPEAICLMKKLVQNKIITVKVVDKLENSSLVELIDKSETPHVSVSKVLLDAGFAVGEQSMVTDKPSDVKETSVPLGVEGKVNPLEWTWVELGVDQTVDVVVCVIYSPGEFYCHVLKEDALKKLNDLNKSLAEHCQQKLPNGFKAEIGQPCCAFFAGDGSWYRALVKEILPNGHVKVHFVDYGNIEEVTADELRMISSTFLNLPFQGIRCQLADIQSRNKHWSEEAITRFQMCVAGIKLQARVVEVTENGIGVELTDLSTCYPRIISDVLIDEHLVLKSASPHKDLPNDRLVNKHELQVHVQGLQAEQWKTIELPVDKTIQANVLEIISPNLFYALPKGMPENQEKLCMLTAELLEYCNAPKSRPPYRPRIGDACCAKYTSDDFWYRAVVLGTSDTDVEVLYADYGNIETLPLCRVQPITSSHLALPFQIIRCSLEGLMELNGSSSQLIIMLLKNFMLNQNVMLSVKGITKNVHTVSVEKCSENGTVDVADKLVTFGLAKNITPQRQSALNTEKMYRMNCCCTELQKQVEKHEHILLFLLNNSTNQNKFIEMKKLLKS

SEQ ID NO: 50 - protein, 495 aa, Homo sapiens
  MISC_FEATURE (1)..(495): CEP63 isoform c
MEALLEGIQNRGHGGGFLTSCEAELQELMKQIDIMVAHKKSEWEGRTHALETCLKIREQELKSLRSQLDVTHKEVGMLHQQVEEHEKIKQEMTMEYKQELKKLHEELCILKRSYEKLQKKQMREFRGNTKNHREDRSEIERLTAKIEEFRQKSLDWEKQRLIYQQQVSSLEAQRKALAEQSEIIQAQLVNRKQKLESVELSSQSEIQHLSSKLERANDTICANELEIERLTMRVNDLVGTSMTVLQEQQQKEEKLRESEKLLEALQEEKRELKAALQSQENLIHEARIQKEKLQEKVKATNTQHAVEAISLESVSATCKQLSQELMEKYEELKRMEAHNNEYKAEIKKLKEQILQGEQSYSSALEGMKMEISHLTQELHQRDITIASTKGSSSDMEKRLRAEMQKAEDKAVEHKEILDQLESLKLENRHLSEMVMKLELGLHECSLPVSPLGSIATRFLEEEELRSHHILERLDAHIEELKRESEKTVRQFTALK

SEQ ID NO: 51 - protein, 455 aa, Homo sapiens
  MISC_FEATURE (1)..(455): IRAK4 isoform a
MNKPITPSTYVRCLNVGLIRKLSDFIDPQEGWKKLAVAIKKPSGDDRYNQFHIRRFEALLQTGKSPTSELLFDWGTTNCTVGDLVDLLIQNEFFAPASLLLPDAVPKTANTLPSKEAITVQQKQMPFCDKDRTLMTPVQNLEQSYMPPDSSSPENKSLEVSDTRFHSFSFYELKNVTNNFDERPISVGGNKMGEGGFGVVYKGYVNNTTVAVKKLAAMVDITTEELKQQFDQEIKVMAKCQHENLVELLGFSSDGDDLCLVYVYMPNGSLLDRLSCLDGTPPLSWHMRCKIAQGAANGINFLHENHHIHRDIKSANILLDEAFTAKISDFGLARASEKFAQTVMTSRIVGTTAYMAPEALRGEITPKSDIYSFGVVLLEIITGLPAVDEHREPQLLLDIKEEIEDEEKTIEDYIDKKMNDADSTSVEAMYSVASQCLHEKKNKRPDIKKVQQLLQ

SEQ ID NO: 52 - protein, 1883 aa, Homo sapiens
  MISC_FEATURE (1)..(1883): TMEM131
MGKRAGGGATGATTAAVSTSAGAGLEPAAARSGGPRSAAAGLLGALHLVMTLVVAAARAEKEAFVQSESIIEVLRFDDGGLLQTETTLGLSSYQQKSISLYRGNCRPIRFEPPMLDFHEQPVGMPKMEKVYLHNPSSEETITLVSISATTSHFHASFFQNRKILPGGNTSFDVVFLARVVGNVENTLFINTSNHGVFTYQVFGVGVPNPYRLRPFLGARVPVNSSFSPIINIHNPHSEPLQVVEMYSSGGDLHLELPTGQQGGTRKLWEIPPYETKGVMRASFSSREADNHTAFIRIKTNASDSTEFIILPVEVEVTTAPGIYSSTEMLDFGTLRTQDLPKVLNLHLLNSGTKDVPITSVRPTPQNDAITVHFKPITLKASESKYTKVASISFDASKAKKPSQFSGKITVKAKEKSYSKLEIPYQAEVLDGYLGFDHAATLFHIRDSPADPVERPIYLTNTFSFAILIHDVLLPEEAKTMFKVHNFSKPVLILPNESGYIFTLLFMPSTSSMHIDNNILLITNASKFHLPVRVYTGFLDYFVLPPKIEERFIDFGVLSATEASNILFAIINSNPIELAIKSWHIIGDGLSIELVAVERGNRTTIISSLPEFEKSSLSDQSSVTLASGYFAVFRVKLTAKKLEGIHDGAIQITTDYEILTIPVKAVIAVGSLTCFPKHVVLPPSFPGKIVHQSLNIMNSFSQKVKIQQIRSLSEDVRFYYKRLRGNKEDLEPGKKSKIANIYFDPGLQCGDHCYVGLPFLSKSEPKVQPGVAMQEDMWDADWDLHQSLFKGWTGIKENSGHRLSAIFEVNTDLQKNIISKITAELSWPSILSSPRHLKFPLTNTNCSSEEEITLENPADVPVYVQFIPLALYSNPSVFVDKLVSRFNLSKVAKIDLRTLEFQVFRNSAHPLQSSTGFMEGLSRHLILNLILKPGEKKSVKVKFTPVHNRTVSSLIIVRNNLTVMDAVMVQGQGTTENLRVAGKLPGPGSSLRFKITEALLKDCTDSLKLREPNFTLKRTFKVENTGQLQIHIETIEISGYSCEGYGFKVVNCQEFTLSANASRDIIILFTPDFTASRVIRELKFITTSGSEFVFILNASLPYHMLATCAEALPRPNWELALYIIISGIMSALFLLVIGTAYLEAQGIWEPFRRRLSFEASNPPFDVGRPFDLRRIVGISSEGNLNTLSCDPGHSRGFCGAGGSSSRPSAGSHKQCGPSVHPHSSHSNRNSADVENVRAKNSSSTSSRTSAQAASSQSANKTSPLVLDSNTVTQGHTAGRKSKGAKQSQHGSQHHAHSPLEQHPQPPLPPPVPQPQEPQPERLSPAPLAHPSHPERASSARHSSEDSDITSLIEAMDKDFDHHDSPALEVFTEQPPSPLPKSKGKGKPLQRKVKPPKKQEEKEKKGKGKPQEDELKDSLADDDSSSTTTETSNPDTEPLLKEDTEKQKGKQAMPEKHESEMSQVKQKSKKLLNIKKEIPTDVKPSSLELPYTPPLESKQRRNLPSKIPLPTAMTSGSKSRNAQKTKGTSKLVDNRPPALAKFLPNSQELGNTSSSEGEKDSPPPEWDSVPVHKPGSSTDSLYKLSLQTLNADIFLKQRQTSPTPASPSPPAAPCPFVARGSYSSIVNSSSSSDPKIKQPNGSKHKLTKAASLPGKNGNPTFAAVTAGYDKSPGGNGFAKVSSNKTGFSSSLGISHAPVDSDGSDSSGLWSPVSNPSSPDFTPLNSFSAFGNSFNLTGEVFSKLGLSRSCNQASQRSWNEFNSGPSYLWESPATDPSPSWPASSGSPTHTATSVLGNTSGLWSTTPFSSSIWSSNLSSALPFTTPANTLASIGLMGTENSPAPHAPSTSSPADDLGQTYNPWRIWSPTIGRRSSDPWSNSHFPHEN

SEQ ID NO: 53 - protein, 807 aa, Homo sapiens
  MISC_FEATURE (1)..(807): EPC2
MSKLSFRARALDAAKPLPIYRGKDMPDLNDCVSINRAVPQMPTGMEKEEESEHHLQRAISAQQVFREKKESMVIPVPEAESNVNYYNRLYKGEFKQPKQFIHIQPFNLDNEQPDYDMDSEDETLLNRLNRKMEIKPLQFEIMIDRLEKASSNQLVTLQEAKLLLNEDDYLIKAVYDYWVRKRKNCRGPSLIPQIKQEKRDGSTNNDPYVAFRRRTEKMQTRKNRKNDEASYEKMLKLRREFSRAITILEMIKRREKTKRELLHLTLEVVEKRYHLGDYGGEILNEVKISRSEKELYATPATLHNGNHHKVQECKTKHPHHLSLKEEASDVVRQKKKYPKKPKAEALITSQQPTPETLPVINKSDIKQYDFHSSDEDEFPQVLSPVSEPEEENDPDGPCAFRRRAGCQYYAPRLDQANHSCENSELADLDKLRYRHCLTTLTVPRRCIGFARRRIGRGGRVIMDRISTEHDPVLKQIDPEMLNSFSSSSQTIDFSSNFSRTNASSKHCENRLSLSEILSNIRSCRLQCFQPRLLNLQDSDSEECTSRKPGQTVNNKRVSAASVALLNTSKNGISVTGGITEEQFQTHQQQLVQMQRQQLAQLQQKQQSQHSSQQTHPKAQGSSTSDCMSKTLDSASAHFAASAVVSAPVPSRSEVAKEQNTGHNNINGVVQPSGTSKTLYSTNMALSSSPGISAVQLVRTVGHTTTNHLIPALCTSSPQTLPMNNSCLTNAVHLNNVSVVSPVNVHINTRTSAPSPTALKLATVAASMDRVPKVTPSSAISSIARENHEPERLGLNGIAETTVAMEVT

SEQ ID NO: 54 - protein, 617 aa, Homo sapiens
  MISC_FEATURE (1)..(617): ZNF813
MALPQGLLTFRDVAIEFSQEEWKCLDPAQRTLYRDVMLENYRNLVSLDISSKCMMKEFSSTAQGNREVIHTGTLQRHESHHTGDFRFQEIDKDIHNLEFQWQEDERNSHEAPMTEIKKLTGSADRYDQRHAGNKPIKDQLGSSFHSHLPELHMFQTQGKIGNQVEKSINDASSISTSQRISCRPKTHISNNYGNNFRNSSLLTQKQEVHMREKSFQCNESGKAFNYSSLLRKHQIIHLGEKQYKCDVCGKVFNRKRNLVCHRRCHTGEKPYRCNECGKTFSQTYSLTCHRRLHTGEKPYKCEECDKAFSFKSNLKRHRRIHAGEKPYKCNECGKTFSQTSSLTCHRRLHTGEKPFKCNECGKTFSRKSSLTCHHRLHTGEKPYKCNECGKTFSQELTLKCHRRLHTGEKPYKCNECGKVFNKKANLARHHRLHSGEKPYKCTECVKTFSRNSALVIHKAIHIGEKRYKCNECGKTFSRISALVIHTAIHTGEKPYKCNECGKGFNRKTHLACHHRLHTGEKPYKCNECGKVFNRKTHLAHHHRLHTGDKPYKCNECGKVFNQKAHLARHHRLHTGEKPYKCNECGKVFNQKANLARHHRLHTGEKPYKFNECGKAFN

SEQ ID NO: 55 - protein, 741 aa, Homo sapiens
  MISC_FEATURE (1)..(741): ZBTB20 isoform 1
MLERKKPKTAENQKASEENEITQPGGSSAKPGLPCLNFEAVLSPDPALIHSTHSLTNSHAHTGSSDCDISCKGMTERIHSINLHNFSNSVLETLNEQRNRGHFCDVTVRIHGSMLRAHRCVLAAGSPFFQDKLLLGYSDIEIPSVVSVQSVQKLIDFMYSGVLRVSQSEALQILTAASILQIKTVIDECTRIVSQNVGDVFPGIQDSGQDTPRGTPESGTSGQSSDTESGYLQSHPQHSVDRIYSALYACSMQNGSGERSFYSGAVVSHHETALGLPRDHHMEDPSWITRIHERSQQMERYLSTTPETTHCRKQPRPVRIQTLVGNIHIKQEMEDDYDYYGQQRVQILERNESEECTEDTDQAEGTESEPKGESFDSGVSSSIGTEPDSVEQQFGPGAARDSQAEPTQPEQAAEAPAEGGPQTNQLETGASSPERSNEVEMDSTVITVSNSSDKSVLQQPSVNTSIGQPLPSTQLYLRQTETLTSNLRMPLTLTSNTQVIGTAGNTYLPALFTTQPAGSGPKPFLFSLPQPLAGQQTQFVTVSQPGLSTFTAQLPAPQPLASSAGHSTASGQGEKKPYECTLCNKTFTAKQNYVKHMFVHTGEKPHQCSICWRSFSLKDYLIKHMVTHTGVRAYQCSICNKRFTQKSSLNVHMRLHRGEKSYECYICKKKFSHKTLLERHVALHSASNGTPPAGTPPGARAGPPGVVACTEGTTYVCSVCPAKFDQIEQFNDHMRMHVSDG